(19) United States
(12) Patent Application Publication
(10) Pub. No.: US 2010/0099673 A1
(43) Pub. Date: Apr. 22, 2010

(54) DECAHYDROQUINOLINE ANALOGS AS CB2 RECEPTOR MODULATORS, USEFUL IN THE TREATMENT OF PAIN, RESPIRATORY AND NON-RESPIRATORY DISEASES

(76) Inventors: Mark T. Bilodeau, Lansdale, PA (US); Nathan R. Kett, Perkiomenville, PA (US); Craig Lindsley, Brentwood, TN (US); Peter J. Manley, Harleysville, PA (US); Jeffrey Y. Melamed, North Wales, PA (US); Daniel Vincent Paone, Lansdale, PA (US); B. Wesley Trotter, Glenside, PA (US); Zhicai Wu, Montvale, NJ (US)

Correspondence Address: MERCK AND CO., INC, P O BOX 2000, RAHWAY, NJ, 07065-0907, US

(21) Appl. No.: 12/523,192

(22) PCT Filed: Jan. 11, 2008
(86) PCT No.: PCT/US08/00404

§ 371 Date: Jul. 15, 2009

Related U.S. Application Data
(60) Provisional application No. 60/880,817, filed on Jan. 17, 2007.

(51) Int. Cl. C07D 215/02; A61K 31/47; C07D 401/02; A61K 31/497; A61K 31/498; A61K 31/5377; C07D 413/02; A61P 29/00; A61P 19/10; A61P 9/00; A61P 37/00; A61P 19/02

(52) U.S. Cl. 514/235.2; 546/156; 514/312; 514/256; 544/335; 544/353; 514/249; 514/255.05; 544/407; 544/128; 514/248

(57) ABSTRACT

The present invention relates to compounds represented by Formula (I): and pharmaceutically acceptable salts thereof. The present invention also provides pharmaceutical compositions comprising the instant compounds. This invention further provides methods to treat and prevent pain, respiratory and non-respiratory diseases.

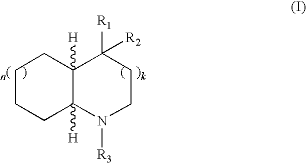

### FIELD OF THE INVENTION

This invention relates to compounds useful as cannibinoid receptor modulators. More particularly, this invention relates to compounds that are CB2 modulators. Even more particularly, this invention relates to compounds that are selective CB2 agonists. The compounds of the invention are useful in the treatment pain and an array of respiratory and non-respiratory diseases, as further discussed infra.

### BACKGROUND OF THE INVENTION

Cannabinoids are psychoactive natural products present in Cannabis sativa L. and have been used as therapeutic agents for thousands of years. They have been shown to have myriad effects in humans, notably in the central nervous system and the cardiovascular system. The therapeutic utility of cannabis is significantly limited due to adverse central effects. The effects of cannabinoids have been shown to occur through their action on two G-protein coupled receptors. A first receptor, CB1, is primarily a centrally-expressed receptor with more limited expression in a variety of peripheral sites, and is believed to be primarily responsible for the central effects of cannabinoids. A second receptor, CB2, is preferentially expressed in the periphery, primarily in cells of the immune system, although it has been identified in central locations to a lesser extent. CB2, expressed in immune cells such as T cells, B cells, macrophages and mast cells, has been shown to have a specific role in mediating immune and inflammatory responses. Given the role of the CB2 receptor in immunomodulation, it is an attractive target for chronic inflammatory pain. CB2 modulators also may have a role in the treatment of osteoporosis, atheroschlerosis, immune disorders, arthritis and other pathological conditions, as discussed infra.

The effects of cannabinoids are due to interaction with specific high affinity receptors, coupled to G proteins, present at the central level (Devane et al., Molecular Pharmacology (1988), 34, 605-613) and the peripheral level (Nye et al., J. Pharmacol. and Exp. Ther. (1985), 234, 784-791; Kaminski et al., Molecular Pharmacol. (1992), 42, 736-742; Munro et al., Nature (1993), 365, 61-65).

The central effects of cannabinoids relate to a first type of cannabinoid receptor (CB1) which is present mainly in the brain but also in the periphery. Munro et al. [Nature, (1993) 365, 61-65] have cloned a second type of cannabinoid receptor, CB2, which is present in the periphery and more particularly on cells of immune origin. The presence of CB2 cannabinoid receptors on lymphoid cells may explain the immunomodulation mentioned above exerted by agonists for cannabinoid receptors.

The psychotropic effects of cannabinois as well as their influence on immune function has been described. [HOLLISTER L. E., J. Psychoact. Drugs, 24 (1992), 159-164]. Most of the in vitro studies have shown immunosuppressant effects for cannabinoids: the inhibition of the proliferative responses in T lymphocytes and B lymphocytes induced by mitogens [Luo, Y. D. et al., Int. J. Immunopharmacol., (1992) 14, 49-56, Schwartz, H. et al., J. Neuroimmunol., (1994) 55, 107-115], the inhibition of the activity of cytotoxic T cells [Klein et al., J. Toxicol. Environ. Health, (1991) 32, 465-477], the inhibition of the microbiocidal activity of macrophages and of the synthesis of TNF-α. [Arata, S. et al., Life Sci., (1991) 49, 473-479; Fisher-Stenger et al., J. Pharm. Exp. Ther., (1993) 267, 1558-1565], the inhibition of the cytolytic activity and of the production of TNF-α. of large granular lymphocytes [Kusher et al., Cell. Immun., (1994) 154, 99-108]. In some studies, amplification effects were observed: increase in the bioactivity of interleukin-1 by mice resident macrophages or differentiated macrophage cell lines, due to increased levels of TNF-α. [Zhu et al., J. Pharm. Exp. Ther., (1994) 270, 1334-1339; Shivers, S. C. et al., Life Sci., (1994) 54, 1281-1289].

### SUMMARY OF THE INVENTION

The present invention relates to compounds represented by Formula (I):

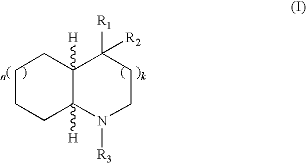

and pharmaceutically acceptable salts thereof. The present invention also provides pharmaceutical compositions comprising the instant compounds. This invention further provides methods to treat and prevent pain, respiratory and non-respiratory diseases.

### DETAILED DESCRIPTION OF THE INVENTION

In one embodiment the present invention relates to compounds represented by Formula (I):

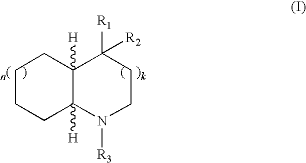

or pharmaceutically acceptable salts and N-oxides thereof, wherein.
m is 1, 2, 3 or 4;
n and k are each independently selected from 0, 1 and 2;
R¹ is independently selected from

(1) H,

(2) OH,

(3) —NH₂,

(4) —NHR⁴,

(5) —NR⁴R⁵,

(6) —CF₃,

(7) —CN,

(8) —C₁₋₆alkyl,

(9) —C₂₋₆alkenyl,

(10) —C₂₋₆alkynyl,

(11) —C₃₋₆cycloalkyl,

(12) heterocycle,

(13) heteroaryl,

(14) aryl,

wherein the alkyl, cycloalkyl, alkenyl and alkynyl of choices (8), (9), (10) and (11) are each independently optionally mono- or di-substituted with substituents independently selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, and
the heterocycle, heteroaryl and aryl of choices (12), (13), and (14), are each optionally mono- or di-substituted with substituents selected from halo, —CN, hydroxy, oxo, —C₁₋₄alkyl, —C₃₋₆cycloalkyl, —CF₃, —CHF₂, —CH₂F, —OC₁₋₆alkyl, —O—CF₃, —O—C₁₋₃alkyl-CF₃, -hydroxyC₁₋₆alkyl, —S(O)₂—R⁶, —C(O)—O—C₁₋₆alkyl, —C(O)—NR⁶R⁷, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —C₁₋₂alkyl-heterocycle, —NR⁶R⁷, —NH—C(O)—R⁶, —NH—C(O)—NR⁶R⁷, —NH—S(O)₂—R⁶, —NH—C₁₋₄alkyl-aryl, and —S—C₁₋₄alkyl, wherein the aryl, heteroaryl and heterocycle portion of the substituents on choices (12), (13) and (14) are each optionally mono or di-substituted with substituents independently selected from halo, —CH₃, —CF₃, —CN, hydroxy and —OC₁₋₄alkyl;

R² is selected from the group consisting of:
(1) —C₁₋₆alkyl,
(2) —C₂₋₆alkenyl,
(3) —C₂₋₆alkynyl,
(4) —C₃₋₆cycloalkyl,
(5) carbocycle,
(6) -aryl,
(7) (CH₂)m-carbocycle,
(8) —(CH₂)m-aryl,
(9) —(CH₂)m-heterocycle,
(10) —(CH₂)m-heteroaryl,
(11) —NHR⁴,
(12) —O—C₁₋₆alkyl,
(13) —N(R⁴)C₁₋₆alkyl,
(14) —O—C₂₋₆alkenyl,
(15) —N(R⁴)C₂₋₆alkenyl,
(16) —O—C₂₋₆alkenyl,
(17) —N(R⁴)C₂₋₆alkynyl,
(18) —O—C₃₋₆cycloalkyl,
(19) —N(R⁴)—C₃₋₆cycloalkyl,
(20) —O-carbocycle,
(21) N(R⁴)-carbocycle,
(22) -heterocycle,
(23) —O-heterocycle,
(24) —N(R⁴)-heterocycle,
(25) -heteroaryl,
(26) —O-heteroaryl,
(27) —N(R⁴)-heteroaryl,
(28) —O-aryl,
(29) —N(aryl)R⁴,
   wherein the alkyl, cycloalkyl, carbocycle, alkenyl and alkynyl of choices (1), (2), (3), (4), (5), (7), (12), (13), (14), (15), (16), (17), (18), (19), (20), and (21) are each optionally mono- or di-substituted with substituents independently selected from hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —O—CF₃, —O—CHF₂, —O—CH₂F, —O—C₁₋₄alkylCF₃, —O—C₁₋₄alkylCHF₂, —O—C₁₋₄alkylCH₂F, -hydroxyC₁₋₄alkyl, —S(O)₂—R⁶, —NR⁴R⁵, —CO₂H, —CO₂R⁴, —C(O)NR⁴R⁵, aryl, heteroaryl, C₃₋₆cycloalkyl and
   wherein the aryl, heteroaryl and heterocycle of choices (6), (8), (9), (10), (22), (23), (24), (25), (26), (27), (28), and (29) are optionally mono-, di- or tri-substituted with substituents independently selected from —CN, hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CHF₃, —O—C₁₋₄alkylCHF₂, —O—C₁₋₄alkylCH₂F, —C₁₋₄alkyl-OH, —S(O)₂—R⁶, —NR⁴R⁵, —CO₂H, CO₂R⁴, —C(O)NR⁴R⁵, aryl, heteroaryl, C₃₋₆cycloalkyl; or
   R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle;

R³ is selected from the group consisting of:
(1) —C₁₋₆alkyl,
(2) —C(O)H,
(3) —S(O)₂H,
(4) —C(O)NHR⁵,
(5) —C(O)—C₁₋₆alkyl,
(6) —S(O)₂—C₁₋₆alkyl,
(7) —C(O)N(R⁵)—C₁₋₆alkyl,
(8) —CH₂—C₂₋₆alkenyl,
(9) —C(O)—C₂₋₆alkenyl,
(10) —S(O)₂—C₂₋₆alkenyl,
(11) —C(O)N(R⁵)—C₂₋₆alkenyl,
(12) —CH₂—C₂₋₆alkynyl,
(13) —C(O)—C₂₋₆alkynyl,
(14) —S(O)₂—C₂₋₆alkynyl,
(15) —C(O)N(R⁵)—C₂₋₆alkynyl,
(16) C₃₋₆cycloalkyl,
(17) —CH₂—C₃₋₆cycloalkyl,
(18) —C(O)—C₃₋₆cycloalkyl,
(19) —S(O)₂—C₃₋₆cycloalkyl,
(20) —C(O)N(R⁵)—C₃₋₆cycloalkyl,
(21) heterocycle,
(22) —CH₂-heterocycle,
(23) —C(O)-heterocycle,
(24) —C(O)—C₁₋₂alkyl-heterocycle,
(25) —S(O)₂-heterocycle,
(26) —C(O)N(R⁵)-heterocycle,
(27) heteroaryl,
(28) —CH₂-heteroaryl,
(29) —C(O)-heteroaryl,
(30) —C(O)—C₁₋₂alkyl-heteroaryl,
(31) —S(O)₂-heteroaryl,
(32) —C(O)N(R⁵)-heteroaryl,
(33) aryl,
(34) —CH₂-aryl,
(35) —C(O)-aryl,
(36) —C(O)—C₁₋₂alkyl-aryl,
(37) —S(O)₂-aryl,
(38) —C(O)N(R⁵)-aryl,
(39) carbocycle,
(40) —CH₂-carbocycle,
(41) —C(O)-carbocycle,
(42) —S(O)₂-carbocycle,
(43) —C(O)N(R₅)-carbocycle,
   wherein the alkyl, cycloalkyl, carbocycle, alkenyl and alkynyl of choices (1), (5), (6), (7), (8), (9), (10), (11), (12), (13), (14), (15), (16), (17) (18) (19), (20), (39), (40), (41), (42) ands (43) are each optionally mono- or di-substituted with substituents independently selected from halo, oxo, hydroxy, —CN, —C₁₋₆alkyl, —CF₃, —CHF₂, CH₂F, —C₁₋₄alkyl-CF₃, —C₁₋₄alkylCHF₂, —C₁₋₄alkylCH₂F, —OC₁₋₆alkyl, —O—CF₃, —C₁₋₄alkylCF₃, hydroxyC₁₋₆alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NH₂, —NH—C(O)—R⁶, —NH—C(O)—C₃₋₆cycloalkyl, —NH—C(O)—C₁₋₄alkyl, —NH—C(O)—NR⁶R⁷, —NH—S(O)₂—R⁶, —S-heteroaryl, —S-heterocycle, aryl, —C(O)-aryl, heteroaryl, —C(O)-heteroaryl, heterocycle, and —C(O)heterocycle, wherein the aryl, heteroaryl, heteroaryl and heterocycle portion of substituents on choices (5) to (19) and (35) to (38) are each optionally mono or di-substituted with substituents independently selected from halo, —CH₃, —CF₃, —CN, hydroxy and —OC₁₋₆alkyl, and
   wherein the aryl, heteroaryl and heterocycle of choices (21), (22), (23), (24), (25), (26), (27), (28), (29), (30), (31), (32), (33), (34), (35), (36), (37) and (38) is optionally mono-, di- or tri-substituted with substituents independently selected from —CN, oxo, —C₁₋₄alkyl, —C₃₋₆cycloalkyl, —CF₃, —CHF₂, —OC₁₋₆alkyl, —O—CF₃, —O—CH₂—CF₃, hydroxy, -hydroxyC₁₋₆alkyl, halo, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —CH₂-heterocycle, —NR⁶R⁷, —NH—C(O)—R⁶, —NH—C(O)—N(CH₃)₂, —NH—S(O)₂—R⁶, —NH—C₁₋₂alkyl-aryl, and —S—C₁₋₃alkyl, wherein the aryl, heteroaryl and heterocycle portion of the substituents on choices (21), (22), (23), (24), (25), (26), (27), (28), (29), (30), (31), (32), (33), (34), (35), (36), (37) and (38) are each optionally mono or di-substituted with substituents independently selected from halo, —CH₃, —CF₃, —CN, hydroxy and —OC₁₋₄alkyl;
   R⁴ and R⁵ are each independently selected from

(1) H,

(2) —CF₃,

(3) —CN,

(4) —C₁₋₆alkyl,

(5) —C₂₋₆alkenyl,

(6) —C₂₋₆alkynyl,

(7) —C₃₋₆cycloalkyl,

(8) heterocycle,

(9) heteroaryl,

(10) aryl,

wherein the alkyl, cycloalkyl, alkenyl and alkynyl of choices (4), (5), (6) and (7) (8) are each independently optionally mono- or di-substituted with substituents independently selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —C(O)—O—C₁₋₆alkyl, —C(O)—NHC₁₋₆alkyl, —C(O)—NR⁶R⁷, —C(O)—O—C(CH₃)₃, —C₃₋₆cycloalkyl, —NH₂, —NHR⁴, —NR⁴R⁵, —NH—C(O)—R⁶, —NH—C(O)—NR⁶R⁷, —NH—S(O)₂—R⁶, and
the heterocycle, heteroaryl and aryl of choices (8), (9) and (10), is each optionally mono- or di-substituted with substituents selected from halo, —CN, hydroxy, oxo, —C₁₋₄alkyl, —C₃₋₆cycloalkyl, —CF₃, —CHF₂, —CH₂F, —OC₁₋₆alkyl, —O—CF₃, —O—C₁₋₃alkyl-CF₃, -hydroxyC₁₋₆alkyl, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, C₁₋₂alkyl-heterocycle, —NR⁶R⁷, —NH—C(O)—NR⁶R⁷, —NH—S(O)₂—R⁶, —NH—C₁₋₄alkyl-aryl, and —S—C₁₋₄alkyl, wherein the aryl, heteroaryl and heterocycle portion of the substituents on choices (9), (10) and (11) are each optionally mono or di-substituted with substituents independently selected from halo, —CH₃, —CF₃, —CN, hydroxy and —OC₁₋₄alkyl;
R⁶ is selected from hydrogen, CF₃, C₁₋₄alkyl, C₃₋₆cycloalkyl, carbocycle, aryl, heterocycle and heteroaryl;
R⁷ is selected from hydrogen and C₁₋₄alkyl;
with the proviso that when R¹ is choice (2), (3), (4) or (5), then R² is other than choice (11), (12), (13), (14), (15), (16), (17), (18), (19), (20), (21), (22), (23), (24), (25), (26), (27), (28), or (29).

Within this embodiment there is a genus wherein

R¹ is independently selected from

(1) H,

(2) OH,

(3) —NH₂,

(4) —NHR⁴,

(5) —NR⁴R⁵,

(6) —CF₃,

(7) —CN,

(8) heterocycle,

(9) heteroaryl,

(10) aryl,

wherein
the heterocycle, heteroaryl and aryl of choices (8), (9), and (10), are each optionally mono- or di-substituted with substituents selected from halo, —CN, —C₁₋₄alkyl, —C₃₋₆cycloalkyl, —CF₃, —OC₁₋₆alkyl, —O—CF₃, —O—C₁₋₃alkyl-CF₃, —C(O)—O—C₁₋₆alkyl, —C(O)—NR⁶R⁷.

Within this embodiment there is a genus wherein

R¹ is independently selected from

(1) H,

(2) OH,

(3) —NH₂,

(6) —CF₃,

(7) —CN.

Within this embodiment there is a genus wherein

R² is selected from the group consisting of:
(1) —C₁₋₆alkyl,
(2) —C₂₋₆alkenyl,
(3) —C₂₋₆alkynyl,
(4) —C₃₋₆cycloalkyl,
(5) carbocycle,
(6) -aryl,
(7) CH₂-carbocycle,
(8) —CH₂-aryl,
(9) —CH₂-heterocycle,
(10) —CH₂-heteroaryl,
(11) —NHR⁴,
(12) —O—C₁₋₆alkyl,
(13) —N(R⁴)C₁₋₆alkyl,
(14) —O—C₂₋₆alkenyl,
(15) —N(R⁴)C₂₋₆alkenyl,
(16) —O—C₂₋₆alkenyl,
(17) —N(R⁴)C₂₋₆alkynyl,
(18) —O—C₃₋₆cycloalkyl,
(19) —N(R⁴)—C₃₋₆cycloalkyl,
(20) —O-carbocycle,
(21) N(R⁴)-carbocycle,
(22) -heterocycle,
(23) —O-heterocycle,
(24) —N(R⁴)heterocycle,
(25) -heteroaryl,
(26) —O-heteroaryl,
(27) —N(R⁴)-heteroaryl,
(28) —O-aryl,
(29) —N(aryl)R⁴,
   wherein the alkyl, cycloalkyl, carbocycle, alkenyl and alkynyl of choices (1), (2), (3), (4), (5), (7), (12), (13), (14), (15), (16), (17), (18), (19), (20), and (21) are each optionally mono- or di-substituted with substituents independently selected from hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —NR⁴R⁵, —CO₂H, CO₂R⁴, —C(O)NR⁴R⁵, aryl, heteroaryl, C₃₋₆cycloalkyl and
   wherein the aryl, heteroaryl and heterocycle of choices (6), (8), (9), (10), (22), (23), (24), (25), (26), (27), (28), and (29) are optionally mono-, di- or tri-substituted with substituents independently selected from —CN, hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —NR⁴R⁵, —CO₂H, CO₂R⁴, —C(O)NR⁴R⁵, aryl, heteroaryl, C₃₋₆cycloalkyl; or
   R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle.

Within this embodiment there is a genus wherein

R² is selected from the group consisting of:
(1) —C₁₋₆alkyl,
(2) —C₂₋₆alkenyl,
(3) —C₂₋₆alkynyl,
(4) —C₃₋₆cycloalkyl,
(5) carbocycle,
(6) -aryl,
(7) CH₂-carbocycle,
(8) —CH₂-aryl,
(9) —CH₂-heterocycle,
(10) —CH₂-heteroaryl,
(11) heterocycle,
(12) heteroaryl,
   wherein the alkyl, cycloalkyl, carbocycle, alkenyl and alkynyl of choices (1), (2), (3), (4), (5) and (7), are each optionally mono- or di-substituted with substituents independently selected from hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O13 CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —NR⁴R⁵, —CO₂H, CO₂R⁴, —C(O)NR⁴R⁵, aryl, heteroaryl, C₃₋₆cycloalkyl and
   wherein the aryl, heteroaryl and heterocycle of choices (6), (8), (9), (10), (11) and (12) are optionally mono-, di- or tri-substituted with substituents independently selected from —CN, hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —NR⁴R⁵, —CO₂H, CO₂R⁴, —C(O)NR⁴R⁵, aryl, heteroaryl, C₃₋₆cycloalkyl; or
   R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle.

Within this embodiment there is a genus wherein

R² is selected from the group consisting of:
(1) —C₁₋₆alkyl,
(2) —C₃₋₆cycloalkyl,
(3) carbocycle,
(4) -aryl,
(5) heterocycle,
(6) heteroaryl,
   wherein the alkyl, cycloalkyl, and carbocycle of choices (1), (2) and (3), are each optionally mono- or di-substituted with substituents independently selected from hydroxy, —CF₃, —C₁₋₄alkylCF₃, aryl, heteroaryl, and
   wherein the aryl, heteroaryl and heterocycle of choices (4), (5) and (6) are optionally mono-, di- or tri-substituted with substituents independently selected from —CN, halo, —CF₃, —CO₂H, CO₂R⁴, —C(O)NR⁴R⁵, aryl, heteroaryl, C₃₋₆cycloalkyl; or
   R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle.

Within this embodiment there is a genus wherein

R³ is selected from the group consisting of:
(1) —C₁₋₆alkyl,
(2) —C(O)H,
(3) —C(O)NHR⁵,
(4) —C(O)—C₁₋₆alkyl,
(5) —C(O)N(R⁵)—C₁₋₆alkyl,
(6) —CH₂—C₂₋₆alkenyl,
(7) —C(O)—C₂₋₆alkenyl,
(8) —C(O)N(R⁵)—C₂₋₆alkenyl,
(9) —CH₂—C₂₋₆alkynyl,
(10) —C(O)—C₂₋₆alkynyl,
(11) —C(O)N(R⁵)—C₂₋₆alkynyl,
(12) C₃₋₆cycloalkyl,
(13) —CH₂—C₃₋₆cycloalkyl,
(14) —C(O)—C₃₋₆cycloalkyl,
(15) —C(O)N(R⁵)—C₃₋₆cycloalkyl,
(16) heterocycle,
(17) —CH₂-heterocycle,
(18) —C(O)-heterocycle,
(19) —C(O)—C₁₋₂alkyl-heterocycle,
(20) —C(O)N(R⁵)-heterocycle,
(21) heteroaryl,
(22) —CH₂-heteroaryl,
(23) —C(O)-heteroaryl,
(24) —C(O)—C₁₋₂alkyl-heteroaryl,
(25) —C(O)N(R⁵)-heteroaryl,
(26) aryl,
(27) —CH₂-aryl,
(28) —C(O)-aryl,
(29) —C(O)—C₁₋₂alkyl-aryl,
(30) —C(O)N(R⁵)-aryl,
(31) carbocycle,
(32) —CH₂-carbocycle,
(33) —C(O)-carbocycle,
(34) —C(O)N(R⁵)-carbocycle,
   wherein the alkyl, cycloalkyl, carbocycle, alkenyl and alkynyl of choices (1), (4), (5), (6), (7), (8), (9), (10), (11), (12), (13), (14), (15), (31), (32), (33), and (34) are each optionally mono- or di-substituted with substituents independently selected from halo, oxo, hydroxy, —CN, —C₁₋₆alkyl, —CF₃, —CHF₂, CH₂F, —C₁₋₄alkyl-CF₃, —C₁₋₄alkylCHF₂, —C₁₋₄alkylCH₂F, —OC₁₋₆alkyl, —O—CF₃, —C₁₋₄alkylCF₃, hydroxyC₁₋₆alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NH₂, —NH—C(O)—R⁶, —NH—C(O)—C₃₋₆cycloalkyl, —NH—C(O)—C₁₋₄alkyl, —NH—C(O)—NR⁶R⁷, —NH—S(O)₂—R⁶, —S-heteroaryl, —S-heterocycle, aryl, —C(O)-aryl, heteroaryl, —C(O)-heteroaryl, heterocycle, and —C(O)heterocycle, wherein the aryl, heteroaryl, heteroaryl and heterocycle portion of substituents on choices (1), (4), (5), (6), (7), (8), (9), (10), (11), (12), (13), (14), (15), (31), (32), (33), and (34) are each optionally mono or di-substituted with substituents independently selected from halo, —CH₃, —CF₃, —CN, hydroxy and —OC₁₋₆alkyl, and
   wherein the aryl, heteroaryl and heterocycle of choices (16), (17), (18), (19), (20), (21), (22), (23), (24), (25), (26), (27), (28), (29) and (30), is optionally mono-, di- or tri-substituted with substituents independently selected from —CN, oxo, —C₁₋₄alkyl, —C₃₋₆cycloalkyl, —CF₃, —CHF₂, —OC₁₋₆alkyl, —O—CF₃, —O—CH₂—CF₃, hydroxy, -hydroxyC₁₋₆alkyl, halo, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —CH₂-heterocycle, —NR⁶R⁷, —NH—C(O)—R⁶, —NH—C(O)—N(CH₃)₂, —NH—S(O)₂—R⁶, —NH—C₁₋₂alkyl-aryl, and —S—C₁₋₃alkyl, wherein the aryl, heteroaryl and heterocycle portion of the substituents on choices (16), (17), (18), (19), (20), (21), (22), (23), (24), (25), (26), (27), (28), (29) and (30) is each optionally mono or di-substituted with substituents independently selected from halo, —CH₃, —CF₃, —CN, hydroxy and —OC₁₋₄alkyl;

Within this genus there is a sub-genus wherein

R³ is selected from the group consisting of:
(1) —C₁₋₆alkyl,
(2) —C(O)—C₁₋₆alkyl,
(3) —CH₂—C₂₋₆alkenyl,
(4) —C(O)—C₂₋₆alkenyl,
(5) —CH₂—C₂₋₆alkynyl,
(6) —C(O)—C₂₋₆alkynyl,
(7) C₃₋₆cycloalkyl,
(8) —C(O)—C₃₋₆cycloalkyl,
(9) —C(O)N(R⁵)—C₃₋₆cycloalkyl,
(10) heterocycle,
(11) —CH₂-heterocycle,
(12) —C(O)-heterocycle,
(13) —C(O)—C₁₋₂alkyl-heterocycle,
(14) heteroaryl,
(15) —CH₂-heteroaryl,
(16) —C(O)-heteroaryl,
(17) —C(O)—C₁₋₂alkyl-heteroaryl,
(18) aryl,
(19) —CH₂-aryl,
(20) —C(O)-aryl,
(21) —C(O)—C₁₋₂alkyl-aryl,
(22) carbocycle,
(23) —CH₂-carbocycle,
(24) —C(O)-carbocycle,
   wherein the alkyl, cycloalkyl, carbocycle, alkenyl and alkynyl of choices (1), (2), (3), (4), (5), (6), (7), (8), (9), (22), (23) and (24) are each optionally mono- or di-substituted with substituents independently selected from halo, oxo, hydroxy, —CN, —C₁₋₆alkyl, —CF₃, —CHF₂, CH₂F, —C₁₋₄alkyl-CF₃, —C₁₋₄alkylCHF₂, —C₁₋₄alkylCH₂F, —OC₁₋₆alkyl, —O—CF₃, —C₁₋₄alkylCF₃, hydroxyC₁₋₆alkyl, —S(O)₂—R⁶, —C(O)—O—C_{1-—}6alkyl, —C(O)—NR⁶R⁷, —C(O)—O—C(CH₃)₃, —C₃₋₆cycloalkyl, —NH₂, —NH—C(O)—R⁶, —NH—C(O)—C₃₋₆cycloalkyl, —NH—C(O)—C₁₋₄alkyl, —NH—C(O)—NR⁶R⁷, —NH—S(O)₂—R⁶, —S-heteroaryl, —S-heterocycle, aryl, —C(O)-aryl, heteroaryl, —C(O)-heteroaryl, heterocycle, and —C(O)heterocycle, wherein the aryl, heteroaryl, heteroaryl and heterocycle portion of substituents on choices (1), (2), (3), (4), (5), (6), (7), (8), (9), (22), (23) and (24) is each optionally mono or di-substituted with substituents independently selected from halo, —CH₃, —CF₃, —CN, hydroxy and —OC₁₋₆alkyl, and
   wherein the aryl, heteroaryl and heterocycle of choices (10), (11), (12), (13), (14), (15), (16), (17), (18), (19), (20) and (21) is optionally mono-, di- or tri-substituted with substituents independently selected from —CN, oxo, —C₁₋₄alkyl, —C₃₋₆cycloalkyl, —CF₃, —CHF₂, —O—C₁₋₆alkyl, —O—CF₃, —O—CH₂—CF₃, hydroxy, -hydroxyC₁₋₆alkyl, halo, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —CH₂-heterocycle, —NR⁶R⁷, —NH—C(O)—R⁶, —NH—C(O)—N(CH₃)₂, —NH—S(O)₂—R⁶, —NH—C₁₋₂alkyl-aryl, and —S—C₁₋₃alkyl, wherein the aryl, heteroaryl and heterocycle portion of the substituents on choices (10), (11), (12), (13), (14), (15), (16), (17), (18), (19), (20) and (21) is each optionally mono or di-substituted with substituents independently selected from halo, —CH₃, —CF₃, —CN, hydroxy and —OC₁₋₄alkyl.

Within this sub-genus there is a class wherein

R³ is selected from the group consisting of:
(1) —C(O)—C₁₋₆alkyl,
(2) —C(O)—C₂₋₆alkenyl,
(3) —C(O)—C₂₋₆alkynyl,
(4) —C(O)—C₃₋₆cycloalkyl,
(5) —C(O)-heterocycle,
(6) —C(O)—C₁₋₂alkyl-heterocycle,
(7) —C(O)-heteroaryl,
(8) —C(O)—C₁₋₂alkyl-heteroaryl,
(9) —C(O)-aryl,
(10) —C(O)—C₁₋₂alkyl-aryl,
(11) —C(O)-carbocycle,
   wherein the alkyl, cycloalkyl, carbocycle, alkenyl and alkynyl of choices (1), (2), (3), (4), and (11), is each optionally mono- or di-substituted with substituents independently selected from oxo, hydroxy, —CN, —CF₃, —C₁₋₄alkyl-CF₃, —C(O)—O—C₁₋₆alkyl, —C(O)—NR⁶_{R}⁷, and
   wherein the aryl, heteroaryl and heterocycle of choices (5), (6), (7), (8), (9) and (10) is optionally mono-, di- or tri-substituted with substituents independently selected from —CN, —C₁₋₄alkyl, —C₃₋₆cycloalkyl, —CF₃, halo, —C(O)—O—C₁₋₆alkyl, —C(O)—NHC₁₋₆alkyl, —C(O)—N(C₁₋₆alkyl)₂, —NR⁶R⁷, —NH—C(O)—R⁶, —NH—C(O)—N(CH₃)₂.

Within this embodiment there is a genus wherein

R⁴ and R⁵ are each independently selected from

(1) H,

(2) —C₁₋₆alkyl,

(3) —C₃₋₆cycloalkyl,

(4) heterocycle,

(5) heteroaryl,

(6) aryl,

wherein the alkyl, cycloalkyl, alkenyl and alkynyl of choices (2) and (3) is each independently optionally mono- or di-substituted with substituents independently selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —C(O)—O—C₁₋₆alkyl, —C(O)—NHC₁₋₆alkyl, —C(O)—NR⁶R⁷, —C(O)—O—C(CH₃)₃, —C₃₋₆cycloalkyl, —NH₂, —NHR⁴, —NR⁴R⁵, —NH—C(O)—R⁶, —NH—C(O)—NR⁶R⁷, —NH—S(O)₂—R⁶, and
the heterocycle, heteroaryl and aryl of choices (4), (5), and (6), are each optionally mono- or di-substituted with substituents selected from halo, —CN, hydroxy, oxo, —C₁₋₄alkyl, —C₃₋₆cycloalkyl, —CF₃, —CHF₂, —CH₂F, —OC₁₋₆alkyl, —O—CF₃, —O—C₁₋₃alkyl-CF₃, -hydroxyC₁₋₃alkyl, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —C₁₋₂alkyl-heterocycle, —NR⁶R⁷, —NH—C(O)—NR⁶R⁷, —NH—S(O)₂—R6, —NH—C₁₋₄alkyl-aryl, and —S—C₁₋₄alkyl, wherein the aryl, heteroaryl and heterocycle portion of the substituents on choices (9), (10) and (11) are each optionally mono or di-substituted with substituents independently selected from halo, —CH₃, —CF₃, —CN, hydroxy and —OC₁₋₄alkyl;

Within this embodiment there is a genus wherein

R⁶ is selected from hydrogen, C₁₋₄alkyl, C₃₋₆cycloalkyl, and aryl.

Within this embodiment there is a genus of formula I wherein

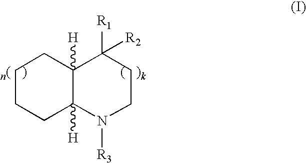

R¹ is independently selected from

(1) H,

(2) OH,

(3) —NH₂,

(4) —NHR⁴,

(5) —NR⁴R⁵,

(6) —CF₃,

(7) —CN,

(8) heterocycle,

(9) heteroaryl,

(10) aryl,

wherein
the heterocycle, heteroaryl and aryl of choices (8), (9), and (10), are each optionally mono- or di-substituted with substituents selected from halo, —CN, —C₁₋₄alkyl, —C₃₋₆cycloalkyl, —CF₃, —OC₁₋₆alkyl, —O—CF₃, —O—C₁₋₃alkyl-CF₃, —C(O)—O—C₁₋₆alkyl, —C(O)—NR⁶R⁷;

R² is selected from the group consisting of:
(1) —C₁₋₆alkyl,
(2) —C₃₋₆cycloalkyl,
(3) carbocycle,
(4) -aryl,
(5) heterocycle,
(6) heteroaryl,
   wherein the alkyl, cycloalkyl, and carbocycle of choices (1), (2) and (3), are each optionally mono- or di-substituted with substituents independently selected from hydroxy, —CF₃, —C₁₋₄alkylCF₃, aryl, heteroaryl, and
   wherein the aryl, heteroaryl and heterocycle of choices (4), (5) and (6) are optionally mono-, di- or tri-substituted with substituents independently selected from —CN, halo, —CF₃, —CO₂H, CO₂R⁴, —C(O)NR⁴R⁵, aryl, heteroaryl, C₃₋₆cycloalkyl; or
   R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle;

R³ is selected from the group consisting of:
(1) —C₁₋₆alkyl,
(2) —C(O)—C₁₋₆alkyl,
(3) —CH₂—C₂₋₆alkenyl,
(4) —C(O)—C₂₋₆alkenyl,
(5) —CH₂—C₂₋₆alkynyl,
(6) —C(O)—C₂₋₆alkynyl,
(7) C₃₋₆cycloalkyl,
(8) —C(O)—C₃₋₆cycloalkyl,
(9) —C(O)N(R⁵)—C₃₋₆cycloalkyl,
(10) heterocycle,
(11) —CH₂-heterocycle,
(12) —C(O)-heterocycle,
(13) —C(O)—C₁₋₂alkyl-heterocycle,
(14) heteroaryl,
(15) —CH₂-heteroaryl,
(16) —C(O)-heteroaryl,
(17) —C(O)—C₁₋₂alkyl-heteroaryl,
(18) aryl,
(19) —CH₂-aryl,
(20) —C(O)-aryl,
(21) —C(O)—C₁₋₂alkyl-aryl,
(22) carbocycle,
(23) —CH₂-carbocycle,
(24) —C(O)-carbocycle,
   wherein the alkyl, cycloalkyl, carbocycle, alkenyl and alkynyl of choices (1), (2), (3), (4), (5), (6), (7), (8), (9), (22), (23) and (24) are each optionally mono- or di-substituted with substituents independently selected from halo, oxo, hydroxy, —CN, —C₁₋₆alkyl, —CF₃, —CHF₂, CH₂F, —C₁₋₄alkyl-CF₃, —C₁₋₄alkylCHF₂, —C₁₋₄alkylCH₂F, —OC₁₋₆alkyl, —O—CF₃, —C₁₋₄alkylCF₃, hydroxyC₁₋₆alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NH₂, —NH—C(O)—R⁶, —NH—C(O)—C₃₋₆cycloalkyl, —NH—C(O)—C₁₋₄alkyl, —NH—C(O)—NR⁶R⁷, —NH—S(O)₂—R⁶, —S-heteroaryl, —S-heterocycle, aryl, —C(O)-aryl, heteroaryl, —C(O)-heteroaryl, heterocycle, and —C(O)heterocycle, wherein the aryl, heteroaryl, heteroaryl and heterocycle portion of substituents on choices (1), (2), (3), (4), (5), (6), (7), (8), (9), (22), (23) and (24) is each optionally mono or di-substituted with substituents independently selected from halo, —CH₃, —CF₃, —CN, hydroxy and —OC₁₋₆alkyl, and
   wherein the aryl, heteroaryl and heterocycle of choices (10), (11), (12), (13), (14), (15), (16), (17), (18), (19), (20) and (21) is optionally mono-, di- or tri-substituted with substituents independently selected from —CN, oxo, —C₁₋₄alkyl, —C₃₋₆cycloalkyl, —CF₃, —CHF₂, —OC₁₋₆alkyl, —O—CF₃, —O—CH₂—CF₃, hydroxy, -hydroxyC₁₋₆alkyl, halo, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —CH₂-heterocycle, —NR⁶R⁷, —NH—C(O)—R⁶, —NH—C(O)—N(CH₃)₂, —NH—S(O)₂—R⁶, —NH—C₁₋₂alkyl-aryl, and —S—C₁₋₃alkyl, wherein the aryl, heteroaryl and heterocycle portion of the substituents on choices (10), (11), (12), (13), (14), (15), (16), (17), (18), (19), (20) and (21) is each optionally mono or di-substituted with substituents independently selected from halo, —CH₃, —CF₃, —CN, hydroxy and —OC₁₋₄alkyl;
   R⁴ and R⁵ are each independently selected from

(1) H,

(2) —C₁₋₆alkyl,

(3) —C₃₋₆cycloalkyl,

(4) heterocycle,

(5) heteroaryl,

(6) aryl,

wherein the alkyl, cycloalkyl, alkenyl and alkynyl of choices (2) and (3) is each independently optionally mono- or di-substituted with substituents independently selected from hydroxy, oxo, halo, —C₁₋₆alkyl, —CF₃, —CHF₂, —CH₂F, —C₁₋₄alkylCF₃, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —C(O)—O—C₁₋₆alkyl, —C(O)—NHC₁₋₆alkyl, —C(O)—NR⁶R⁷, —C(O)—O—C(CH₃)₃, —C₃₋₆cycloalkyl, —NH₂, —NHR⁴, —NR⁴R⁵, —NH—C(O)—R⁶, —NH—C(O)—NR⁶R⁷, —NH—S(O)₂—R⁶, and
the heterocycle, heteroaryl and aryl of choices (4), (5), and (6), are each optionally mono- or di-substituted with substituents selected from halo, —CN, hydroxy, oxo, —C₁₋₄alkyl, —C₃₋₆cycloalkyl, —CF₃, —CHF₂, —CH₂F, —OC₁₋₆alkyl, —O—CF₃, —O—C₁₋₃alkyl-CF₃, -hydroxyC₁₋₆alkyl, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —C₁₋₂alkyl-heterocycle, —NR⁶R⁷, —NH—C(O)—NR⁶R⁷, —NH—S(O)₂—R⁶, —NH—C₁₋₄alkyl-aryl, and —S—C₁₋₄alkyl, wherein the aryl, heteroaryl and heterocycle portion of the substituents on choices (9), (10) and (11) are each optionally mono or di-substituted with substituents independently selected from halo, —CH₃, —CF₃, —CN, hydroxy and —OC₁₋₄alkyl;
R⁶ is selected from hydrogen, C₁₋₄alkyl, C₃₋₆cycloalkyl, and aryl.

Within this genus there is a sub-genus wherein

R¹ is independently selected from

(1) H,

(2) OH,

(3) —NH₂,

(6) —CF₃,

(7) —CN;

R³ is selected from the group consisting of:
(1) —C(O)—C₁₋₆alkyl,
(2) —C(O)—C₂₋₆alkenyl,
(3) —C(O)—C₂₋₆alkynyl,
(4) —C(O)—C₃₋₆cycloalkyl,
(5) —C(O)-heterocycle,
(6) —C(O)—C₁₋₂alkyl-heterocycle,
(7) —C(O)-heteroaryl,
(8) —C(O)—C₁₋₂alkyl-heteroaryl,
(9) —C(O)-aryl,
(10) —C(O)—C₁₋₂alkyl-aryl,
(11) —C(O)-carbocycle,
   wherein the alkyl, cycloalkyl, carbocycle, alkenyl and alkynyl of choices (1), (2), (3), (4), and (11), is each optionally mono- or di-substituted with substituents independently selected from oxo, hydroxy, —CN, —CF₃, —C₁₋₄alkyl-CF₃, —C(O)—O—C₁₋₆alkyl, —C(O)—NR⁶R⁷, and
   wherein the aryl, heteroaryl and heterocycle of choices (5), (6), (7), (8), (9) and (10) is optionally mono-, di- or tri-substituted with substituents independently selected from —CN, —C₁₋₄alkyl, —C₃₋₆cycloalkyl, —CF₃, halo, —C(O)—O—C₁₋₆alkyl, —C(O)—NHC₁₋₆alkyl, —C(O)—N(C₁₋₆alkyl)₂, —NR⁶R⁷, —NH—C(O)—R⁶, —NH—C(O)—N(CH₃)₂.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

As used here a “cycloalkyl”, is a saturated monocyclic hydrocarbon ring.

As used here a “carbocycle”, is a mono cyclic or bi-cyclic carbocyclic non-aromatic ring having at least one double bond.

The term “aryl”, unless specifically stated otherwise, refers to single and multi-cyclic aromatic ring systems in which the ring members are all carbon, for example, phenyl or naphthyl.

The term “heteroaryl”, unless specifically stated otherwise, refers to single and multi-cyclic aromatic ring systems in which at least one of the ring members is other than carbon. Heteroaryl includes, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, and the like.

The term “heterocycle”, unless specifically stated otherwise, refers to single and multi-cyclic non-aromatic ring systems in which at least one of the ring members is other than carbon. Heterocycle includes pyrrolidine, piperidine, piperazine, morpholine, azetidine, tetrahydropyran, tetrahydrofuran, dioxane, and the like.

The term “amine” unless specifically stated otherwise includes primary, secondary and tertiary amines.

The term “halogen” includes fluorine, chlorine, bromine and iodine atoms.

The term “oxide” of heteroaryl groups is used in the ordinary well-known chemical sense and include, for example, N-oxides of nitrogen heteroatoms.

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above compounds of the invention may be shown without a definitive stereochemistry at certain positions. The present invention includes all stereoisomers of the compounds of the invention and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “aryl”, unless specifically stated otherwise, refers to single and multi-cyclic aromatic ring systems in which the ring members are all carbon, for example, phenyl or naphthyl.

The term “heteroaryl”, unless specifically stated otherwise, refers to single and multi-cyclic aromatic ring systems in which at least one of the ring members is other than carbon. Heteroaryl includes, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, and the like.

The term “heterocycle”, unless specifically stated otherwise, refers to single and multi-cyclic non-aromatic ring systems in which at least one of the ring members is other than carbon. Heterocycle includes pyrrolidine, piperidine, piperazine, morpholine, azetidine, tetrahydropyran, tetrahydrofuran, dioxane, and the like.

The term “optionally substituted” is intended to include both substituted and unsubstituted. Thus, for example, optionally substituted aryl can represent a pentafluorophenyl or a phenyl ring. Further, the substitution can be made at any of the groups. For example, substituted aryl(C₁₋₆)alkyl includes substitution on the aryl group as well as substitution on the alkyl group.

The term “polycyclic ring” means more than 3 fused rings and includes carbon as ring atoms. The polycyclic ring can be saturated or unsaturated. The polycyclic ring can be unsubstituted, singly substituted or, if possible, multiply substituted, with substituent groups in any possible position. The individual rings may or may not be of the same type. Examples of polycyclic rings include adamantane, bicyclooctane, norbornane and bicyclononanes.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

“Pharmaceutically acceptable non-toxic acids”, including inorganic and organic acids, salts prepared from, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The pharmaceutical compositions of the present invention comprise a compound represented of the invention (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles and the like. Further, the compositions can be in a form suitable for use in transdermal devices. These formulations may be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, may also be prepared in powder or liquid concentrate form.

A formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms can generally contain between from about 1 mg to about 1000 mg of the active ingredient.

The conditions recited herein can be treated or prevented by the administration of from about 0.01 mg to about 140 mg of the instant compounds per kilogram of body weight per day.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy. For example, inflammatory pain may be effectively treated by the administration of from about 0.01 mg to about 75 mg of the present compound per kilogram of body weight per day, or alternatively about 0.5 mg to about 3.5 g per patient per day. Neuropathic pain may be effectively treated by the administration of from about 0.01 mg to about 125 mg of the present compound per kilogram of body weight per day, or alternatively about 0.5 mg to about 5.5 g per patient per day.

It is understood that compounds of this invention can be administered at prophylactically effective dosage levels to prevent the above-recited conditions, as well as to prevent other conditions mediated through CB2 receptor.

The Compounds of the invention may be used with other therapeutic agents such as those described below. Such other therapeutic agent(s) may be administered prior to, simultaneously with, or following the administration of the cannabinoid receptor modulators in accordance with the invention.

Compounds of the invention may be used in combination with other drugs that are used in the treatment/prevention/suppression or amelioration of the diseases or conditions for which compounds of the invention are useful. Such other drugs may be administered, by a route and in an amount commonly used therefor, contemporaneously or sequentially with a compound of the invention. When a compound of the invention is used contemporaneously with one or more other drugs, a pharmaceutical composition containing such other drugs in addition to the compound of the invention is preferred. Accordingly, the pharmaceutical compositions of the present invention include those that also contain one or more other active ingredients, in addition to a compound of the invention. Examples of active ingredients that may be combined with a compound of the invention, either administered separately or in the same pharmaceutical compositions, include, but are not limited to: (1) non-steroidal anti-inflammatory agents, such as ibuprofen and naproxen; (2) COX-2 inhibitors, such as Celebrex and Arcoxia; (3) bradykinin B1 receptor antagonists; (4) sodium channel blockers and antagonists; (5) nitric oxide synthase (NOS) inhibitors; (6) glycine site antagonists; (7) potassium channel openers; (8) AMPA/kainate receptor antagonists; (9) calcium channel antagonists; (10) GABA-A receptor modulators (e.g., a GABA-A receptor agonist); (11) matrix metalloprotease (MMP) inhibitors; (12) thrombolytic agents; (13) opioids such as morphine; (14) neutrophil inhibitory factor (NIF); (15) L-Dopa; (16) carbidopa; (17) levodopa/carbidopa; (18) dopamine agonists such as bromocriptine, pergolide, pramipexole, ropinirole; (19) anticholinergics; (20) amantadine; (21) carbidopa; (22) catechol O-methyltransferase (“COMT”) inhibitors such as entacapone and tolcapone; (23) Monoamine oxidase B (“MAO-B”) inhibitors; (24) opiate agonists or antagonists; (25) 5HT receptor agonists or antagonists; (26) NMDA receptor agonists or antagonists; (27) NK1 antagonists; (28) selective serotonin reuptake inhibitors (“SSRI”) and/or selective serotonin and norepinephrine reuptake inhibitors (“SSNRI”); (29) tricyclic antidepressant drugs, (30) norepinephrine modulators; (31) lithium; (32) valproate; and (33) neurontin (gabapentin).

Additional examples of active ingredients that may be combined with a compound of the invention, either administered separately or in the same pharmaceutical compositions, include, but are not limited to: (34) cyclosporins (e.g., cyclosporin A); (35) CTLA4-Ig, antibodies such as anti-ICAM-3, anti-IL-2 receptor (Anti-Tac), anti-CD45RB, anti-CD2, anti-CD3 (OKT-3), anti-CD4, anti-CD80, anti-CD86, and monoclonal antibody OKT3; (36) agents blocking the interaction between CD40 and gp39, such as antibodies specific for CD40 and/or gp39 (i.e., CD154); (37) fusion proteins constructed from CD40 and gp39 (CD40Ig and CD8gp39), (38) inhibitors, such as nuclear translocation inhibitors of NF-kappa B function, such as deoxyspergualin (DSG); (38) steroids such as prednisone or dexamethasone; (39) gold compounds; (40) antiproliferative agents such as methotrexate, FK506 (tacrolimus, Prograf), mycophenolate mofetil; (41) cytotoxic drugs such as azathiprine and cyclophosphamide; (42) TNF-α. inhibitors such as tenidap; (43) anti-TNF antibodies or soluble TNF receptor such as etanercept (Enbrel); (44) rapamycin (sirolimus or Rapamune); (45) leflunomide (Arava); (46) anticytokines such as antiIL-4 or IL-4 receptor fusion proteins and PDE 4 inhibitors such as Ariflo, and (47) the PTK inhibitors disclosed in the following U.S. patent applications, incorporated herein by reference in their entirety: Ser. No. 09/097,338, filed Jun. 15, 1998; Ser. No. 09/094,797, filed Jun. 15, 1998; Ser. No. 09/173,413, filed Oct. 15, 1998; and Ser. No. 09/262,525, filed Mar. 4, 1999. See also the following documents and references cited therein and incorporated herein by reference: Hollenbaugh, D., Et Al, “Cleavable CD40Ig Fusion Proteins and the Binding to Sgp39”, J. Immunol. Methods (Netherlands), 188(1), pp. 1-7 (Dec. 15, 1995); Hollenbaugh, D., et al, “The Human T Cell Antigen Gp39, A Member of the TNF Gene Family, Is a Ligand for the CD40 Receptor: Expression of a Soluble Form of Gp39 with B Cell Co-Stimulatory Activity”, EMBO J. (England), 11(12), pp. 4313-4321 (December 1992); and Moreland, L. W. et al., “Treatment of Rheumatoid Arthritis with a Recombinant Human Tumor Necrosis Factor Receptor (P75)-Fc Fusion Protein,” New England J. of Medicine, 337(3), pp. 141-147 (1997).

The above other therapeutic agents, when employed in combination with the compounds of the present invention, may be used, for example, in those amounts indicated in the Physicians' Desk Reference (PDR) or as otherwise determined by one of ordinary skill in the art.

Thus, compounds of the invention may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them.

Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and laminating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of the invention may also be useful in the treatment of inflammation, for example in allergies, asthma, autoimmune diseases such as transplant rejection (e.g., kidney, heart, lung, liver, pancreas, skin; host versus graft reaction (HVGR), graft versus host reaction (GVHR) etc.), rheumatoid arthritis, and amyotrophic lateral sclerosis, T-cell mediated autoimmune diseases such as multiple sclerosis, psoriasis and Sjogren's syndrome, Type II inflammatory diseases such as vascular inflammation (including vasculitis, arteritis, atherosclerosis and coronary artery disease), diseases of the central nervous system such as stroke, pulmonary diseases such as bronchitis obliteraus and primary pulmonary hypertension, and solid, delayed Type IV hypersensitivity reactions, and hematologic malignancies such as leukemia and lymphomas.

Compounds of the invention may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflammation.

Compounds of the invention may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions.

Compounds of the invention may also be useful in the treatment of cancer, including but not limited to adenomas, meningiomas, glioblastomas and melanoma.

The preferred uses of CB2 agonists are for the treatment of pain and inflammatory conditions. Pain is selected from inflammatory pain, viseral pain, cancer pain, neuropathic pain, lower back pain, muscular skeletal, post operative pain, acute pain, migraine and inflammatory pain associated with rheumatoid arthritis or osteoarthritis. Indications associated with inflammation include allergies, asthma, multiple sclerosis, vasculitis, arteritis, atherosclerosis and coronary artery disease.

Compounds of the invention are effective for treating and preventing pain, respiratory and non-respiratory diseases.

Respiratory diseases for which the compounds of the invention are useful include but are not limited to chronic pulmonary obstructive disorder, emphysema, asthma, and bronchitis. Compounds of the invention are also useful in the treatment and prevention of indications disclosed in European Patent Documents Nos. EP 0570920 and EP 0444451; International Publications Nos. WO 97/29079, WO 99/02499, WO 98/41519, and WO 9412466; U.S. Pat. Nos. 4,371,720, 5,081,122, 5,292,736, and 5,013,387; and French Patent No. FR 2735774.

The compounds of the invention stimulate inhibitory pathways in cells, particularly in leukocytes, lung epithelial cells, or both, and are thus useful in treating respiratory diseases. “Leukocyte activation” is defined herein as any or all of cell proliferation, cytokine production, adhesion protein expression, and production of inflammatory mediators. “Epithelial cell activation” is defined herein as the production of any or all of mucins, cytokines, chemokines, and adhesion protein expression.

The Compounds of the invention are expected to block the activation of lung epithelial cells by moieties such as allergic agents, inflammatory cytokines or smoke, thereby limiting release of mucin, cytokines, and chemokines. Another preferred embodiment of the present invention comprises use of novel cannabinoid receptor modulator compounds to treat respiratory disease wherein the compounds selectively inhibit lung epithelial cell activation.

Thus, Compounds of the invention, in treating leukocyte activation-associated disorders are useful in treating a range of disorders such as: transplant (such as organ transplant, acute transplant, xenotransplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; respiratory and pulmonary diseases including but not limited to chronic obstructive pulmonary disease (COPD), emphysema, bronchitis, and acute respiratory distress syndrome (ARDS); inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (asthma, hayfever, allergic rhinitis) or skin allergies; scleracierma; mycosis fungoides; acute inflammatory and respiratory responses (such as acute respiratory distress syndrome and ischemia/reperfusion injury); dermatomyositis; alopecia greata; chronic actinic dermatitis; eczema; Behcet's disease; Pustulosis palmoplanteris; Pyoderma gangrenum; Sezary's syndrome; atopic dermatitis; systemic sclerosis; and morphea. The term “leukocyte activation-associated” or “leukocyte-activation mediated” disease as used herein includes each of the above referenced diseases or disorders. In a particular embodiment, the compounds of the present invention are useful for treating the aforementioned exemplary disorders irrespective of their etiology. The combined activity of the present compounds towards monocytes, macrophages, T-cells, etc. may be useful in treating any of the above-mentioned disorders.

Exemplary non-respiratory cannabinoid receptor-mediated diseases include transplant rejection, rheumatoid arthritis, multiple sclerosis, inflammatory bowel disease, lupus, graft v. host disease, T-cell mediated hypersensitivity disease, psoriasis, Hashimoto's thyroiditis, Guillain-Barre syndrome, cancer, contact dermatitis, allergic rhinitis, and ischemic or reperfusion injury.

Compounds of the invention also inhibit the Fc gamma dependent production of TNF-α in human monocytes/macrophages. The ability to inhibit Fc gamma receptor dependent monocyte and macrophage responses results in additional anti-inflammatory activity for the present compounds. This activity is especially of value, for example, in treating inflammatory diseases such as arthritis or inflammatory bowel disease. In particular, the present compounds are useful for treating autoimmune glomerulonephritis and other instances of glomerulonephritis induced by deposition of immune complexes in the kidney that trigger Fc gamma receptor responses leading to kidney damage.

Cannabinoid receptors may be expressed on gut epithelial cells and hence regulate cytokine and mucin production and may be of clinical use in treating inflammatory diseases related to the gut. Cannabinoid receptors are also expressed on lymphocytes, a subset of leukocytes. Thus, cannabinoid receptor modulators will inhibit B and T-cell activation, proliferation and differentiation. Thus, such compounds will be useful in treating autoimmune diseases that involve either antibody or cell mediated responses such as multiple sclerosis and lupus.

In addition, cannabinoid receptors regulate the Fc epsilon receptor and chemokine induced degranulation of mast cells and basophils. These play important roles in asthma, allergic rhinitis, and other allergic disease. Fc epsilon receptors are stimulated by IgE-antigen complexes. Compounds of the present invention inhibit the Fc epsilon induced degranulation responses, including the basophil cell line, RBL. The ability to inhibit Fc epsilon receptor dependent mast cell and basophil responses results in additional anti-inflammatory and anti-allergic activity for the present compounds. In particular, the present compounds are useful for treating asthma, allergic rhinitis, and other instances of allergic disease.

The utility of the compounds of the invention can be demonstrated by the following assays.

### Cyclic AMP assay

Chinese Hamster Ovary cells (CHO) expressing human CB1 or human CB2 (3.3×10⁵ cells/ml) were preincubated for 15 min at room temperature with tested agonist and 3-isobutyl-1-methylxanthine (IBMX; 200 μM) in phosphate buffered saline containing 1 mg/ml BSA (assay buffer) followed by 30 min incubation with forskolin in a total volume of 10 μl. The optimal forskolin concentration for each cell line was established in a separate experiment and adjusted to stimulate 70% of maximal cAMP response. cAMP content was measured using an HTRF assay (CisBio) according to the manufacturer's two step protocol.

In this assay, compounds of the invention have an IP ranging from 1 nM to >17000 nM. The Examples below have an IP ranging from 1 nM to >17000 nM.

| | | CB2 | CB2 | CB1 |
|---|---|---|---|---|
| | | (HTRF) | Emax | (HTRF) |
| Example | Structure | nM | (%) | nM |
| 131 | 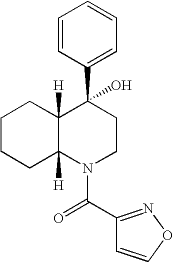 | 37 | 100 | >17000 |
| 135 | 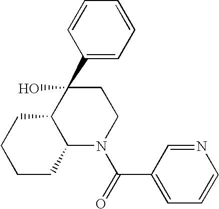 | 10 | 100 | 6778 |
| 258 | 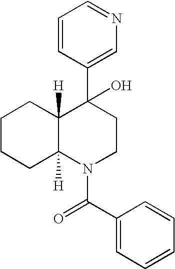 | 938 | 86 | nd |
| 260 | 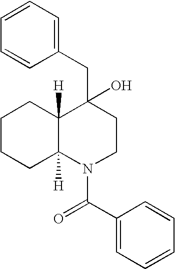 | 90 | 93 | nd |
| 271 | 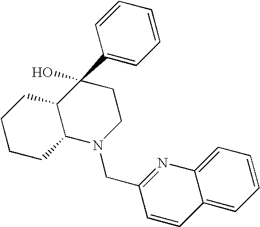 | 85 | 75 | >17000 |
| 274 | 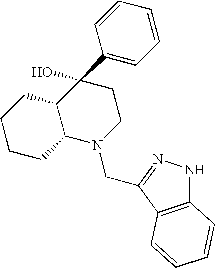 | 19 | 102 | >17000 |
| 285 | 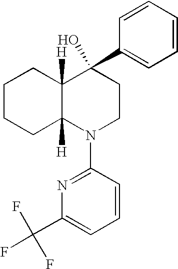 | 6 | 96 | >17000 |
| 287 | 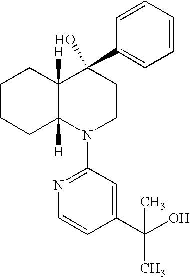 | 1.3 | 99 | 10500 |
| 291 | 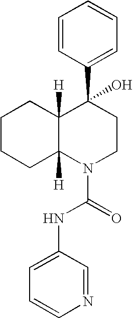 | 1105 | 76 | >17000 |
| 289 | 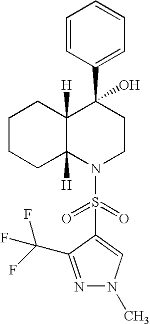 | 41 | 101 | >17000 |

### Evaluation of Compounds in the Rat CFA Inflammatory Pain Model and Rat Iodoacetate Model of Osteoarthritis

### Rat Complete Freunds Adjuvant (CFA) Model of Inflammatory Pain

This model is used to determine the efficacy of test compounds against acute inflammatory pain produced by intradermal injection of Complete Freunds adjuvant (CFA) into a hind paw. Male Sprague Dawley rats (150-200 g; Taconic) are tested for baseline mechanical hind paw withdrawal thresholds by wrapping the rat in a towel and placing the hind paw (either left or right) in a modified Randal-Sellito paw pinch apparatus (Stoelting, Wood Dale, Ill.). A plastic plinth is placed on the plantar aspect of the hind paw and an increasing force (measured in grams) is applied to the hind paw. The test is terminated when the rat vocalizes or pulls its hind paw away from the plinth. The rat's hind paw withdrawal threshold (gm.) is recorded at that point. The mechanical stimulus is applied to each hind paw 3 times at each testing time point, and average mechanical hind paw withdrawal thresholds are determined for both the left and right hind paw. A maximal hind paw withdrawal threshold of 450 gm. is used to avoid tissue damage. Following determination of pre-CFA nociceptive thresholds, rats receive an intradermal injection of CFA (100 ul, 1 mg/ml) into the plantar aspect of the left hind paw and are subsequently returned to their cages in the animal holding room where they are maintained on soft bedding. In this model of acute inflammation, the inflammation develops over a 24 hour period, at which time edema and redness of the affected hind paw is observed (Stein et al. Pharmacol Biochem Behav 31:455, 1988). 24 hours following CFA injection, rats are tested for decreased mechanical paw withdrawal thresholds (mechanical hypersensitivity). Effects of the test compound on CFA-induced mechanical hypersensitivity are determined by dosing the test compound, vehicle and naproxen (20 mg/kg, p.o.; positive control) in different groups of rats and testing mechanical hind paw withdrawal thresholds at various times post-dosing depending on the pharmacokinetic properties of the test compound (n=8-10/group). Efficacy in the CFA model is evaluated by determining the % reversal of mechanical hypersensitivity using the formula:

$$\% reversal = \frac{( post \text{-} drug threshold - post \text{-} CFA threshold )}{( pre \text{-} CFA threshold - post \text{-} CFA threshold )} \times 100$$

At the conclusion of the experiment, all rats are immediately euthanized by CO₂.

### Rat Iodoacetate Model of Osteoarthritis Pain

This model is used to evaluate the efficacy of test compounds against chronic osteoarthritic pain produced by intraarticular injection of iodoacetate into a knee joint. Male Sprague Dawley rats (200-300 g; Taconic) are placed in individual plastic chambers on an elevated mesh galvanized steel platform and allowed to acclimate for approximately 60 min. Rats are then tested for baseline mechanical paw withdrawal thresholds by applying a series of calibrated von Frey filaments (0.25-15 g) to the left hind paw and determining the median withdrawal threshold using the Dixon “up-down” method (Chaplan et al., J Neurosci Meth 53:55, 1994). Pre-iodoacetate mechanical hind paw withdrawal thresholds are determined, and rats having a threshold <15 g are excluded from the study. Additionally, hind paw weight bearing is measured using an incapacitance instrument. Rats are tested for hind paw weight bearing by placing the animal in a Plexiglas box (approximately 4″ width, 4″ height, 5″ length) such that the posterior half of the animal is loosely restrained. This box is placed on an incapacitance analgesia meter (Stoelting Co.) such that the rats hind paws are positioned on two mechano-transducers that measure weight bearing (g) on each paw. Rats remain in this box for a period of ˜60 sec. during which average weight bearing on each hind paw is measured and displayed via LCD readout. Following determination of baseline pain related behaviors, rats are briefly anesthetized using isoflurane (1-5% to effect, inhalation) and receive an intraarticular injection of monosodium iodoacetate (2 mg/25 ul) into the left hind limb knee joint. Rats are continuously monitored until full recovery from the anesthetic (<5 min) and are subsequently returned to their cages where they are maintained on soft bedding. Intraarticular injection of iodoacetate has been found to produce degeneration of joint cartilage which is maximum at day 21, although the rats do not exhibit changes in body weight or locomotor activity and are found to be in otherwise good health (Fernihough et al. Pain 112:83, 2004). In-house results have demonstrated that mechanical hypersensitivity (von Frey filaments) and decreased weight bearing (incapacitance instrument) persists for >8 weeks following iodoacetate injection. 6 weeks following iodoaceteate injection, rats are tested for these pain-related behaviors. Effects of test compound on iodoacetate-induced mechanical hypersensitivity and decreased weight bearing are determined by dosing the test compound, vehicle and naproxen (20 mg/kg, p.o.; positive control) in different groups of rats and testing mechanical hind paw withdrawal thresholds and weight bearing at various times post-dosing depending on the pharmacokinetic properties of the test compound (n=8-10/group). Efficacy in the iodoacetate model is evaluated by determining the % reversal of mechanical hypersensitivity and weight bearing using the formula:

$$\% reversal = \frac{( \begin{matrix}post \text{-} drug threshold - \\ post \text{-} iodoacetate threshold\end{matrix} )}{( \begin{matrix}pre \text{-} iodoacetate threshold - \\ post \text{-} iodoacetate threshold\end{matrix} )} \times 100$$

At the conclusion of the experiment, all rats are immediately euthanized by CO₂.

### Methods of Synthesis

Several methods for preparing the compounds of this invention are illustrated in the following Examples. Starting materials and the requisite intermediates are in some cases commercially available, or can be prepared according to literature procedures or as illustrated herein. All ¹H NMR spectra were obtained on instrumentation at a field strength of 400 or 500 MHz.

The abbreviations used hereinunder are as follows unless specified otherwise:

4-MeBnOH 4-Methylbenzyl alcohol

### CDI 1,1′-Carbonyldiimidazole

### TEA Triethylamine

TBSCl t-Butyldimethylsilyl chloride

### DMF Dimethylformamide

(+)-BINAP (+)-2,2′-Bis(diphenylphosphino)-1,1′-binaphthyl
NaOtBu Sodium t-butoxide

### DIPEA Diisopropylethylamine

EtOAc Ethyl acetate
TBSOTf t-Butyldimethylsilyl triflate
TBS t-butyldimethylsilyl

### THF Tetrahydrofuran

### DMAP 4-Dimethylaminopyridine

RT Room temperature

### h Hours

### min Minutes

### DCM Dichloromethane

### MeCN Acetonitrile

iPrOH 2-Propanol
n-BuOH 1-Butanol
EDC 1-(3-Dimethylaminopropyl)-3-ethylcarbodiimide hydrochloride
HOAt 1-Hydroxy-7-azabenzotriazole

### INTERMEDIATES AND EXAMPLES

The following examples are provided so that the invention might be more fully understood. These examples are illustrative only and should not be construed as limiting the invention in any way.

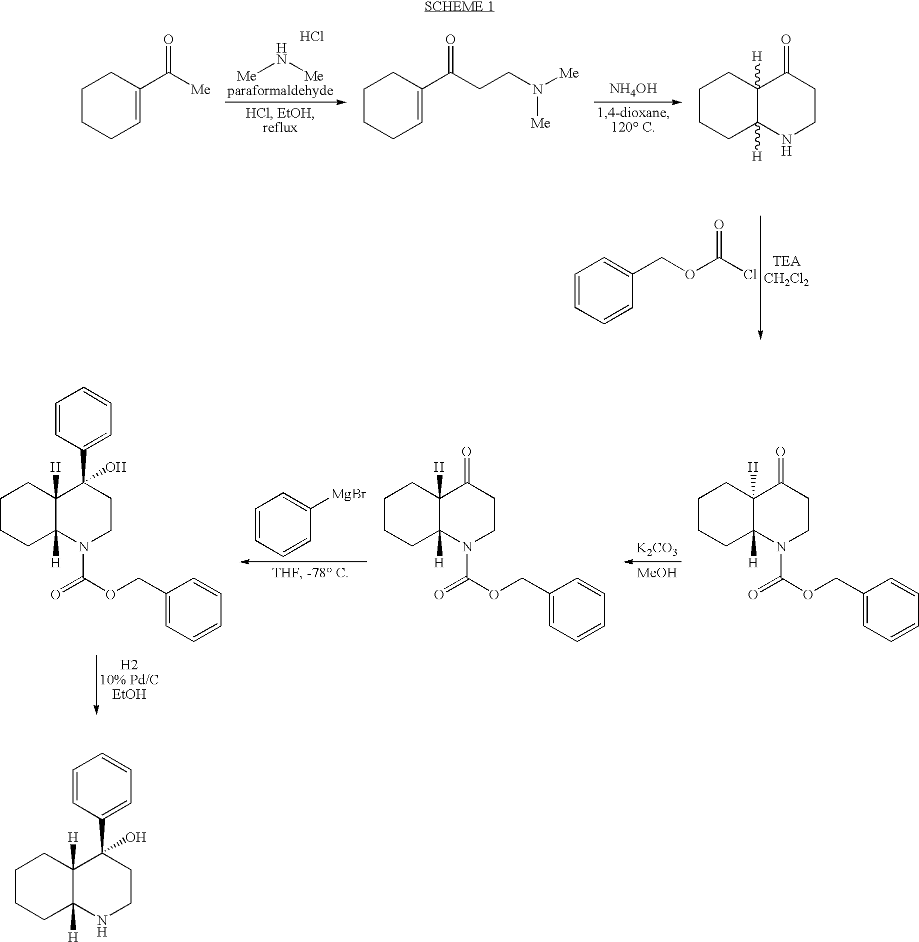

### 1. 1-Cyclohex-1-en-1-yl-3-(dimethylamino)propan-1-one

1-Acetyl-1-cyclohexene (21 mL, 163 mmol), dimethylamine hydrochloride (14.65 g, 180 mmol), paraformaldehyde (9.81 g, 327 mmol), 37% aqueous HCl (1.3 mL, 15.83 mmol) were combined in EtOH (65 mL) then heated to reflux. After 20 hr the mixture was cooled to RT and concentrated. The residue was taken up in H₂O and washed with Et₂O (3×). The aqueous layer was made basic with NaOH pellets (pH 10) and extracted with CHCl₃ (6×). The combined organic layers were dried (MgSO₄), filtered, and concentrated to give the title compound as a light orange oil (17.9 g, 60%): ¹H-NMR (CDCl₃, 500 MHz) δ 6.93 (s, 1H), 2.85 (t, J=7.08 Hz, 2H), 2.66 (m, 2H), 2.28 (s, 6H), 2.22 (m, 4H), 1.62 (m, 4H).

### 2. Racemic-benzyl (cis)-4-oxooctahydroquinoline-1(2H)-carboxylate

1-Cyclohex-1-en-1-yl-3-(dimethylamino)propan-1-one (17.9 g, 99 mmol) was combined with 1,4-dioxane (40 mL) and concentrated NH₄OH (40 mL) in a stainless steel pressure vessel. The vessel was sealed then heated in an oil bath at 120° C. After 16 hr the mixture was cooled to RT and concentrated. The residue was taken up in CH₂Cl₂, dried (MgSO₄), filtered, and concentrated to give octahydroquinolin-4(1H)-one as an amber oil (14.77 g, 98%). The crude material was used immediately in the next step without purification.

To a solution of crude octahydroquinolin-4(1H)-one (14.77 g, 96 mmol) in CH₂Cl₂ (300 mL) was added TEA (20 mL, 143 mmol). The mixture was cooled to 0° C. then benzyl chloroformate (18 mL, 126 mmol) was added slowly. After 2 hr benzyl chloroformate (2.0 mL) was added and stirring continued. After 90 min the mixture was concentrated. The residue was taken up in EtOAc, filtered through a pad of Celite, and concentrated to give benzyl (trans)-4-oxooctahydroquinoline-1(2H)-carboxylate as an amber oil (30.68 g). The crude material was used immediately in the next step without purification. ¹H-NMR (CDCl₃, 500 MHz) δ7.35 (m, 5H), 5.15 (AB, J=12.45 and 7.33 Hz, 2H), 4.33 (m, 1H), 3.58 (m, 1H), 3.51 (m, 1H), 2.56-2.36 (m, 4H), 2.08 (m, 1H), 1.81 (m, 2H), 1.41-1.20 (m, 4H).

Crude benzyl 4-oxooctahydroquinoline-1(2H)-carboxylate (30.68 g, 107 mmol) was taken up in MeOH (400 mL) to which was added K₂CO₃ (44.3 g, 320 mmol) at RT. After 3 hr the mixture was filtered and concentrated. The residue was taken up in H₂O and extracted with CH₂Cl₂ (3×). The combined organic layers were washed with brine, dried (MgSO₄), filtered, and concentrated. Flash column (1.5 Kg silica gel, gradient, 0-20% EtOAc/hexanes) gave the title compound as a pale yellow oil (9.09 g, 30%): ¹H-NMR (CDCl₃, 500 MHz) [symbol] 7.34 (m, 5H), 5.18 (s, 2H), 4.71-4.47 (bm, 1H), 4.26 (bs, 1H), 3.45 (m, 1H), 2.75 (bs, 1H), 2.48 (bs, 1H), 2.33 (bm, 2H), 1.76 (bm, 2H), 1.48 (bs, 1H), 1.31 (bm, 4H).

### 3. Racemic-benzyl (cis)-4-hydroxy-4-phenyloctahydroquinoline-1(2H)-carboxylate

To a solution of racemic-benzyl (cis)-4-oxooctahydroquinoline-1(2H)-carboxylate (25 g, 87 mmol) in dry THF (350 mL) was added phenylmagnesium bromide (87 mL, 261 mmol) slowly at −78° C. During the addition a thick gummy precipitate formed making stirring difficult. The mixture was allowed to warm as the bath warmed to RT. After 16 hr the mixture was cooled to 0° C., diluted with saturated aqueous NH₄Cl and extracted with ethyl acetate (3×). The combined organic layers were dried (MgSO₄), filtered, and concentrated. Flash column (750 g silica gel, gradient, 0-15% EtOAc/hexanes) gave the title compound as an oil which solidified under vacuum (23.46 g, 74%): ¹H-NMR (CDCl₃, 500 MHz) δ7.4 (m, 10H), 5.18 (s, 2H), 4.6-3.9 (bm, 2H), 3.42 (m, 1H), 2.6-1.1 (m, 12H).

### 4. (cis)-4-phenyldecahydroquinolin-4-ol (single enantiomer)

Benzyl (cis)-4-hydroxy-4-phenyloctahydroquinoline-1(2H)-carboxylate (enantiomer A from step 6, 100 mg, 0.274 mmol) was hydrogenated (balloon) with 10% Pd/C (29 mg, 0.027 mmol) in EtOH (10 mL) at RT. After 6 hr the mixture filtered through a pad of Celite washing with ethanol and concentrated to give the title compound as an off-white foam (65 mg, 100%). ¹H-NMR (CDCl₃, 500 MHz) [symbol] 7.58 (m, 2H), 7.39 (m, 2H), 7.3 (m, 1H), 3.22 (m, 1H), 2.85 (m, 2H), 2.3 (m, 2H), 2.0-1.2 (m, 10H).

### 5. Separation of the enantiomers of racemic-(cis)-4-phenyldecahydroquinolin-4-ol

Separation of the enantiomers (A and B) of the title compound was achieved using normal-phase chiral HPLC on a Chiracel OD (10×50 cm, 20 μm) column eluting with 20% IPA in hexanes containing 0.1% DEA at 225 mL/min. Analytical HPLC (Chiracel OD, 4.6×250 mm, 10 μm, 20% IPA in hexanes containing 0.1% DEA at 1 mL/min) retention times A: 8.5 min and B: 9.4 min.

### 6. Separation of the enantiomers of racemic-benzyl (cis)-4-hydroxy-4-phenyloctahydroquinoline-1(2H)-carboxylate

Separation of the enantiomers (A and B) of the title compound was achieved using chiral SFC-HPLC on a Chiracel OJ-H (21.2×250 mm) column eluting with 25% MeOH/CO₂ at 50 mL/min, 100 bar. Analytical HPLC (Chiracel OD, 4.6×250 mm, 10 μm, 15% EtOH in hexanes containing 0.1% DEA at 1 mL/min) retention times A: 6.17 min and B: 9.18 min. Enantiomer-A corresponds to enantiomer-B of (cis)-4-phenyldecahydroquinolin-4-ol after deprotection.

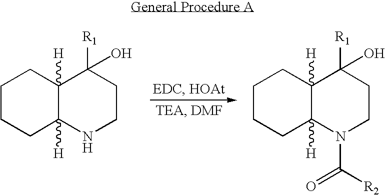

### Example I-1 (General Procedure A)

### (Cis)-1-(isoxazol-3-ylcarbonyl)-4-phenyldecahydroquinolin-4-ol (enantiomer A)

(Cis)-4-phenyldecahydroquinolin-4-ol (enantiomer A, 100 mg, 0.432 mmol), isoxazole-3-carboxylic acid (63.5 mg, 0.562 mmol), EDC (108 mg, 0.562 mmol), HOAt (1.124 ml, 0.5 M in DMF, 0.562 mmol), and TEA (0.078 ml, 0.562 mmol) were combined in DMF (2 ml) at RT. After 16 hr the mixture was filtered using a 0.45 μm PTFE syringe filter and concentrated. The residue was purified by preparative reversed-phase HPLC on a Waters Sunfire column (20×150 mm, 5 μm) with gradient elution using 5-70% CH₃CN/water containing 0.1% TFA over 20 minutes. Fractions containing the product were pooled and concentrated to give the title compound as an off-white solid (72 mg, 51%). ¹H-NMR (CDCl₃, 500 MHz) apparent amide bond rotomers δ 8.47 (s, 1H), 7.44 (m, 2H), 7.37 (t, J=7.08 Hz, 2H), 7.29 (m, 1H), 6.69 (s, 1H), 4.81 (m, 0.5H), 4.69 (m, 0.5H), 4.33 (m, 0.5H), 4.21 (m, 0.5H), 3.81 (dt, J=2.19 and 13.43 Hz, 0.5H), 3.50 (dt, 2.69 and 13.18 Hz, 0.5H), 2.53-2.11 (m, 3H), 1.91-1.14 (m, 8H). HRMS (M+H)⁺ 327.1730.

Compounds in Table I were synthesized using General Procedure A.

TABLE I

| | | | MS |
|---|---|---|---|
| Example | Structure | Name | (M + H)+ |
| 1 | 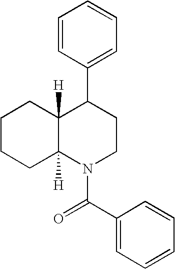 | 1-benzoyl-4- phenyldecahydroquinoline | 320.2008 |
| 2 | 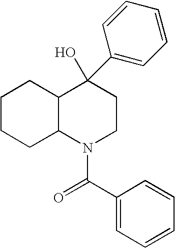 | 1-benzoyl-4- phenyldecahydroquinolin-4-ol | 336.19 |
| 3 | 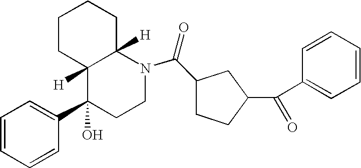 | (3-{[(cis)-4-hydroxy-4- phenyloctahydroquinolin-1(2H)- yl]carbonyl}cyclopentyl)(phenyl) methanone | 432.25 |
| 4 | 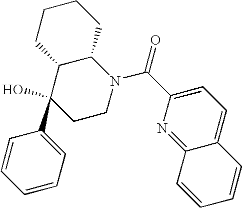 | (cis)-4-phenyl-1-(quinolin-2- ylcarbonyl)decahydroquinolin-4-ol | 387.2 |
| 5 | 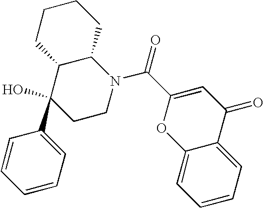 | 2-{[(cis)-4-hydroxy-4- phenyloctahydroquinolin-1(2H)- yl]carbonyl}-4H-chromen-4-one | 404.18 |
| 6 | 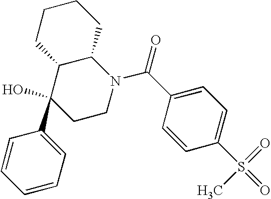 | (cis)-1-[4- (methylsulfonyl)benzoyl]-4- phenyldecahydroquinolin-4-ol | 414.17 |
| 7 | 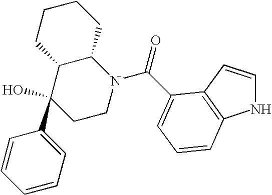 | (cis)-1-(1H-indol-4-ylcarbonyl)-4- phenyldecahydroquinolin-4-ol | 375.2 |
| 8 | 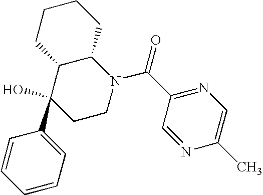 | cis-1-[(5-methylpyrazin-2- yl)carbonyl]-4- phenyldecahydroquinolin-4-ol | 352.2013 |
| 9 | 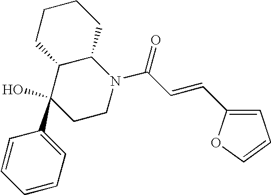 | (cis)-1-[(2E)-3-(2-furyl)prop-2- enoyl]-4- phenyldecahydroquinolin-4-ol | 352.19 |
| 10 | 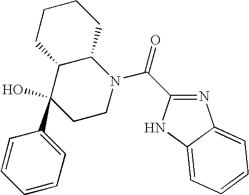 | (cis)-1-(1H-benzimidazol-2- ylcarbonyl)-4- phenyldecahydroquinolin-4-ol | 376.2 |
| 11 | 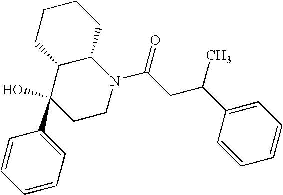 | (cis)-4-phenyl-1-(3- phenylbutanoyl)decahydroquinolin- 4-ol | 378.24 |
| 12 | 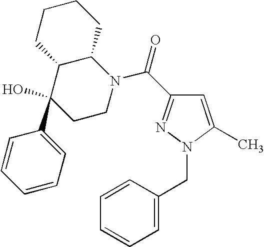 | (cis)-1-[(1-benzyl-5-methyl-1H- pyrazol-3-yl)carbonyl]-4- phenyldecahydroquinolin-4-ol | 430.24 |
| 13 | 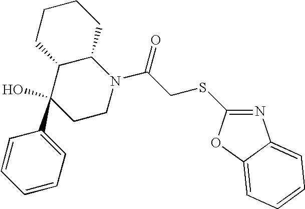 | (cis)-1-[(1,3-benzoxazol-2- ylthio)acetyl]-4- phenyldecahydroquinolin-4-ol | 423.17 |
| 14 | 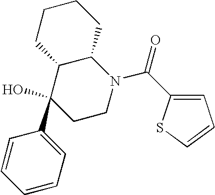 | (cis)-4-phenyl-1-(2- thienylcarbonyl)decahydroquinolin- 4-ol | 342.15 |
| 15 | 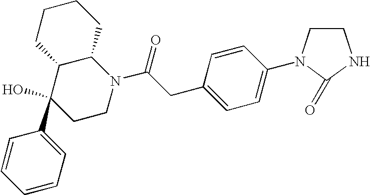 | 1-(4-{2-[(cis)-4-hydroxy-4- phenyloctahydroquinolin-1(2H)- yl]-2- oxoethyl}phenyl)imidazolidin-2- one | 434.24 |
| 16 | 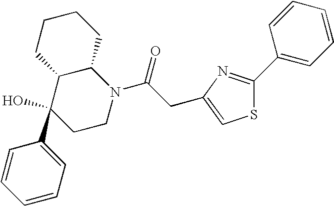 | (cis)-4-phenyl-1-[(2-phenyl-1,3- thiazol-4- yl)acetyl]decahydroquinolin-4-ol | 433.19 |
| 17 | 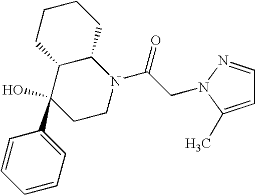 | (cis)-1-[(5-methyl-1H-pyrazol-1- yl)acetyl]-4- phenyldecahydroquinolin-4-ol | 354.21 |
| 18 | 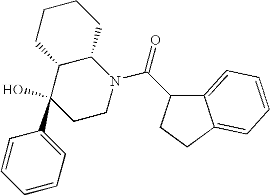 | (cis)-1-(2,3-dihydro-1H-inden-1- ylcarbonyl)-4- phenyldecahydroquinolin-4-ol | 376.22 |
| 19 | 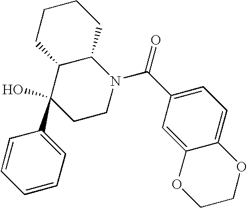 | (cis)-1-(2,3-dihydro-1,4- benzodioxin-6-ylcarbonyl)-4- phenyldecahydroquinolin-4-ol | 394.2 |
| 20 | 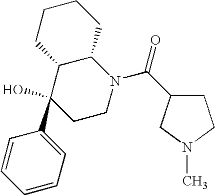 | (cis)-1-[(1-methylpyrrolidin-3- yl)carbonyl]-4- phenyldecahydroquinolin-4-ol | 343.23 |
| 21 | 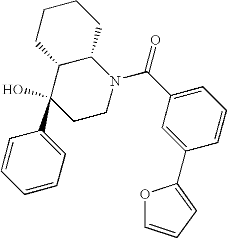 | (cis)-1-[3-(2-furyl)benzoyl]-4- phenyldecahydroquinolin-4-ol | 402.2 |
| 22 | 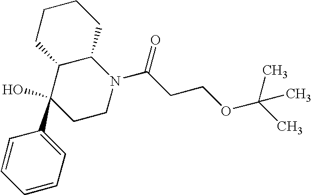 | (cis)-1-(3-tert-butoxypropanoyl)-4- phenyldecahydroquinolin-4-ol | 360.25 |
| 23 | 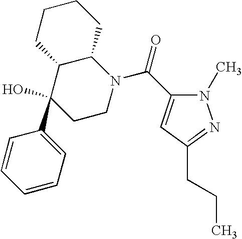 | (cis)-1-[(1-methyl-3-propyl-1H- pyrazol-5-yl)carbonyl]-4- phenyldecahydroquinolin-4-ol | 382.24 |
| 24 | 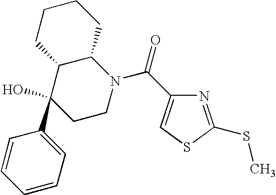 | (cis)-1-{[2-(methylthio)-1,3- thiazol-4-yl]carbonyl}-4- phenyldecahydroquinolin-4-ol | 389.13 |
| 25 | 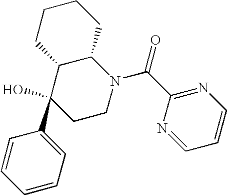 | (cis)-4-phenyl-1-(pyrimidin-2- ylcarbonyl)decahydroquinolin-4-ol | 338.18 |
| 26 | 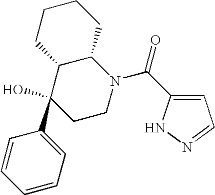 | (cis)-4-phenyl-1-(1H-pyrazol-5- ylcarbonyl)decahydroquinolin-4-ol | 326.18 |
| 27 | 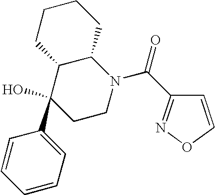 | (cis)-1-(isoxazol-3-ylcarbonyl)-4- phenyldecahydroquinolin-4-ol | 327.17 |
| 28 | 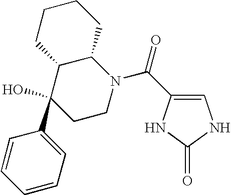 | 4-{[(cis)-4-hydroxy-4- phenyloctahydroquinolin-1(2H)- yl]carbonyl}-1,3-dihydro-2H- imidazol-2-one | 342.18 |
| 29 | 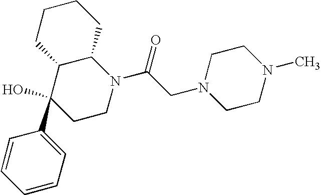 | (cis)-1-[(4-methylpiperazin-1- yl)acetyl]-4- phenyldecahydroquinolin-4-ol | 372.26 |
| 30 | 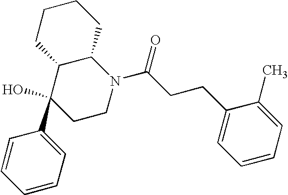 | (cis)-1-[3-(2- methylphenyl)propanoyl]-4- phenyldecahydroquinolin-4-ol | 378.24 |
| 31 | 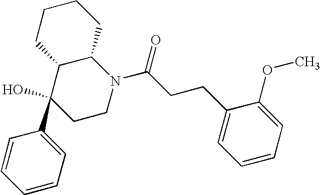 | (cis)-1-[3-(2- methoxyphenyl)propanoyl]-4- phenyldecahydroquinolin-4-ol | 394.23 |
| 32 | 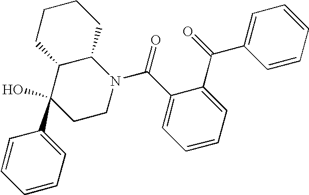 | (2-{[(cis)-4-hydroxy-4- phenyloctahydroquinolin-1(2H)- yl]carbonyl}phenyl)(phenyl)methanone | 440.22 |
| 33 | 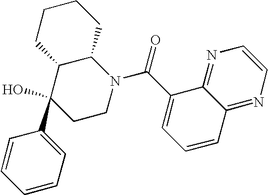 | (cis)-4-phenyl-1-(quinoxalin-5- ylcarbonyl)decahydroquinolin-4-ol | 388.2 |
| 34 | 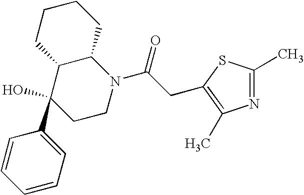 | (cis)-1-[(2,4-dimethyl-1,3-thiazol- 5-yl)acetyl]-4- phenyldecahydroquinolin-4-ol | 385.19 |
| 354 | 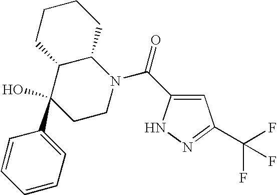 | (cis)-4-phenyl-1-{[3- (trifluoromethyl)-1H-pyrazol-5- yl]carbonyl}decahydroquinolin-4- ol | 394.17 |
| 36 | 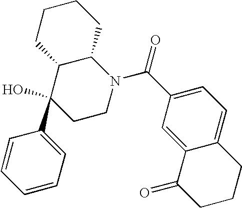 | 7-{[(cis)-4-hydroxy-4- phenyloctahydroquinolin-1(2H)- yl]carbonyl}-3,4- dihydronaphthalen-1(2H)-one | 404.22 |
| 37 | 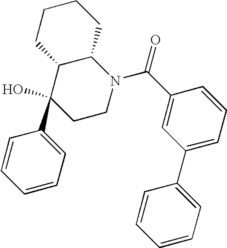 | (cis)-1-(biphenyl-3-ylcarbonyl)-4- phenyldecahydroquinolin-4-ol | 412.22 |
| 38 | 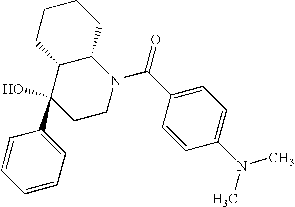 | (cis)-1-[4- (dimethylamino)benzoyl]-4- phenyldecahydroquinolin-4-ol | 379.23 |
| 39 | 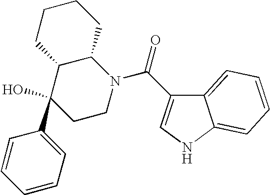 | (cis)-1-(1H-indol-3-ylcarbonyl)-4- phenyldecahydroquinolin-4-ol | 375.2 |
| 40 | 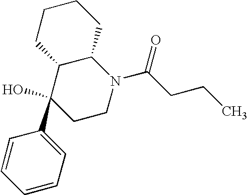 | (cis)-1-butyryl-4- phenyldecahydroquinolin-4-ol | 302.21 |
| 41 | 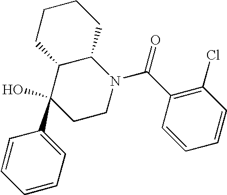 | (cis)-1-(2-chlorobenzoyl)-4- phenyldecahydroquinohn-4-ol | 370.15 |
| 42 | 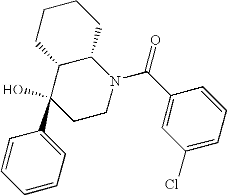 | (cis)-1-(3-chlorobenzoyl)-4- phenyldecahydroquinolin-4-ol | 370.15 |
| 43 | 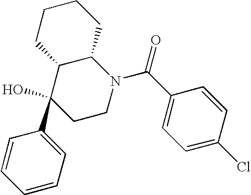 | (cis)-1-(4-chlorobenzoyl)-4- phenyldecahydroquinolin-4-ol | 370.15 |
| 44 | 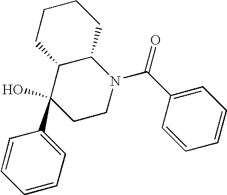 | (cis)-1-benzoyl-4- phenyldecahydroquinolin-4-ol | 336.19 |
| 45 | 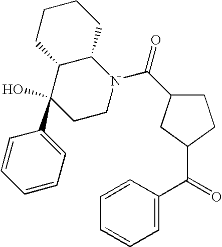 | (3-{[(cis)-4-hydroxy-4- phenyloctahydroquinolin-1(2H)- yl]carbonyl}cyclopentyl)(phenyl) methanone | 432.25 |
| 46 | 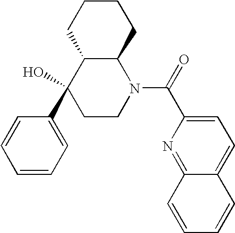 | (trans)-4-phenyl-1-(quinolin-2- ylcarbonyl)decahydroquinolin-4-ol | 387.2 |
| 47 | 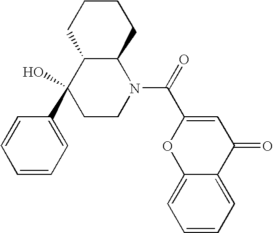 | 2-{[(trans)-4-hydroxy-4- phenyloctahydroquinolin-1(2H)- yl]carbonyl}-4H-chromen-4-one | 404.18 |
| 48 | 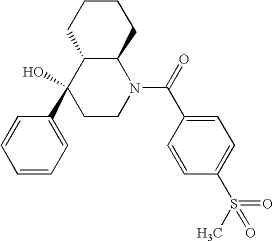 | (trans)-1-[4- (methylsulfonyl)benzoyl]-4- phenyldecahydroquinolin-4-ol | 414.17 |
| 49 | 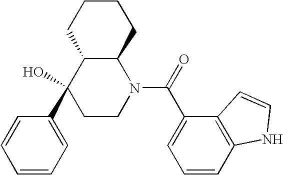 | (trans)-1-(1H-indol-4-ylcarbonyl)- 4-phenyldecahydroquinolin-4-ol | 375.2 |
| 50 | 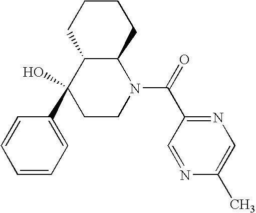 | (trans)-1-[(5-methylpyrazin-2- yl)carbonyl]-4- phenyldecahydroquinolin-4-ol | 352.2 |
| 51 | 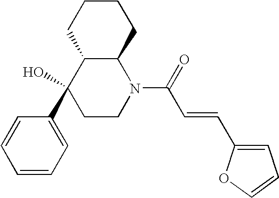 | (trans)-1-[(2E)-3-(2-furyl)prop-2- enoyl]-4- phenyldecahydroquinohn-4-ol | 352.19 |
| 52 | 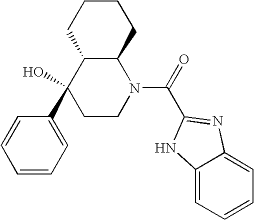 | (trans)-1-(1H-benzimidazol-2- ylcarbonyl)-4- phenyldecahydroquinolin-4-ol | 376.2 |
| 53 | 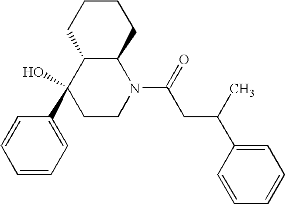 | (trans)-4-phenyl-1-(3- phenylbutanoyl)decahydroquinolin- 4-ol | 378.24 |
| 54 | 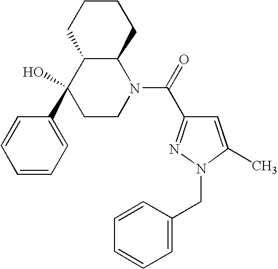 | (trans)-1-[(1-benzyl-5-methyl-1H- pyrazol-3-yl)carbonyl]-4- phenyldecahydroquinolin-4-ol | 430.24 |
| 55 | 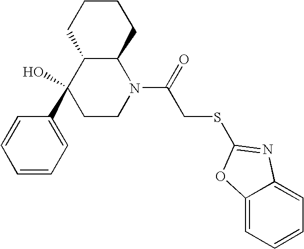 | (trans)-1-[(1,3-benzoxazol-2- ylthio)acetyl[-4- phenyldecahydroquinolin-4-ol | 423.17 |
| 56 | 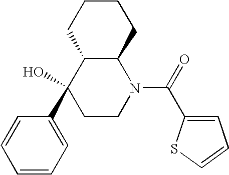 | (trans)-4-phenyl-1-(2- thienylcarbonyl)decahydroquinolin- 4-ol | 342.15 |
| 57 | 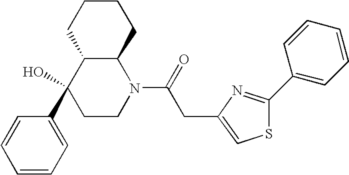 | (trans)-4-phenyl-1-[(2-phenyl-1,3- thiazol-4- yl)acetyl]decahydroquinolin-4-ol | 433.19 |
| 58 | 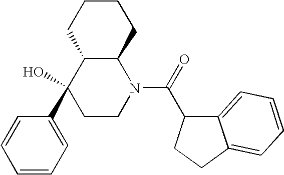 | (trans)-1-(2,3-dihydro-1H-inden-1- ylcarbonyl)-4- phenyldecahydroquinolin-4-ol | 376.22 |
| 59 | 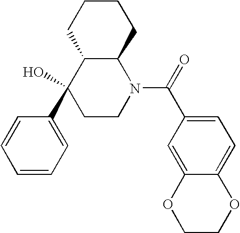 | (trans)-1-(2,3-dihydro-1,4- benzodioxin-6-ylcarbonyl)-4- phenyldecahydroquinolin-4-ol | 394.2 |
| 60 | 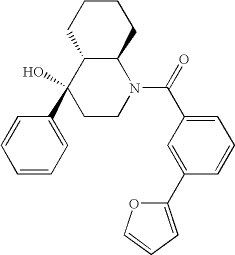 | (trans)-1-[3-(2-furyl)benzoyl]-4- phenyldecahydroquinolin-4-ol | 402.2 |
| 61 | 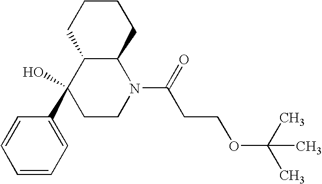 | (trans)-1-(3-tert-butoxypropanoyl)- 4-phenyldecahydroquinolin-4-ol | 360.25 |
| 62 | 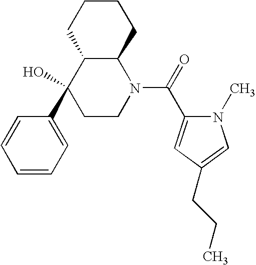 | (trans)-1-[(1-methyl-3-propyl-1H- pyrazol-5-yl)carbonyl]-4- phenyldecahydroquinolin-4-ol | 382.24 |
| 63 | 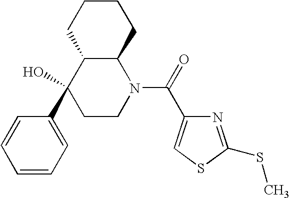 | (trans)-1-{[2-(methylthio)-1,3- thiazol-4-yl]carbonyl}-4- phenyldecahydroquinolin-4-ol | 389.13 |
| 64 | 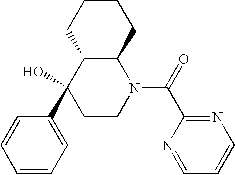 | (trans)-4-phenyl-1-(pyrimidin-2- ylcarbonyl)decahydroquinolin-4-ol | 338.18 |
| 65 | 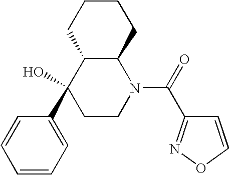 | (trans)-1-(isoxazol-3-ylcarbonyl)- 4-phenyldecahydroquinolin-4-ol | 327.17 |
| 66 | 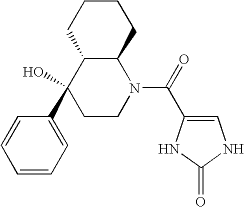 | 4-{[(trans)-4-hydroxy-4- phenyloctahydroquinolin-1(2H)- yl]carbonyl}-1,3-dihydro-2H- imidazol-2-one | 342.18 |
| 67 | 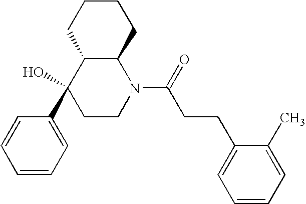 | (trans)-1-[3-(2- methylphenyl)propanoyl]-4- phenyldecahydroquinolin-4-ol | 378.24 |
| 68 | 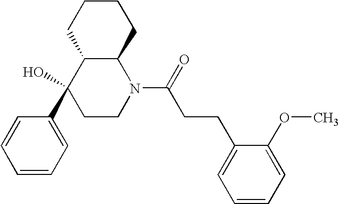 | (trans)-1-[3-(2- methoxyphenyl)propanoyl]-4- phenyldecahydroquinolin-4-ol | 394.23 |
| 69 | 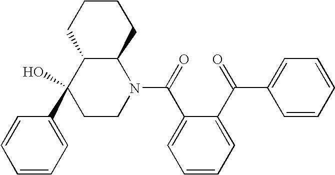 | (2-{[(trans)-4-hydroxy-4- phenyloctahydroquinolin-1(2H)- yl]carbonyl}phenyl)(phenyl)methanone | 440.22 |
| 70 | 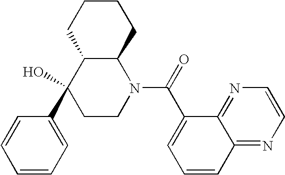 | (trans)-4-phenyl-1-(quinoxalin-5- ylcarbonyl)decahydroquinolin-4-ol | 388.2 |
| 71 | 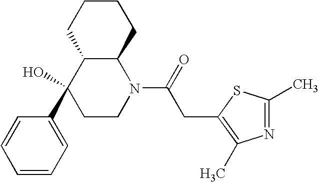 | (trans)-1-[(2,4-dimethyl-1,3- thiazol-5-yl)acetyl]-4- phenyldecahydroquinolin-4-ol | 385.19 |
| 72 | 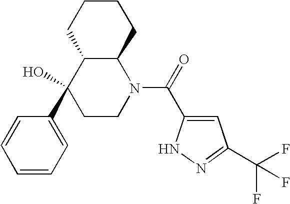 | (trans)-4-phenyl-1-{[3- (trifluoromethyl)-1H-pyrazol-5- yl]carbonyl}decahydroquinolin-4- ol | 394.17 |
| 73 | 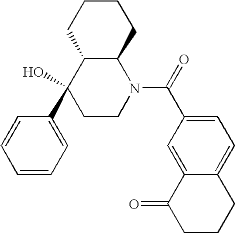 | 7-{[(trans)-4-hydroxy-4- phenyloctahydroquinolin-1(2H)- yl]carbonyl}-3,4- dihydronaphthalen-1(2H)-one | 404.22 |
| 74 | 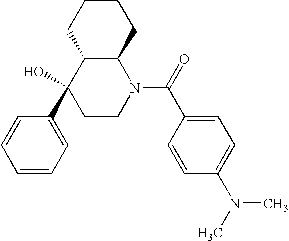 | (trans)-1-[4- (dimethylamino)benzoyl]-4- phenyldecahydroquinolin-4-ol | 379.23 |
| 75 | 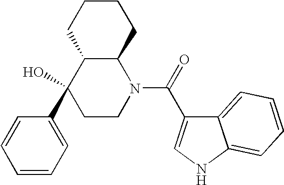 | (trans)-1-(1H-indol-3-ylcarbonyl)- 4-phenyldecahydroquinolin-4-ol | 375.2 |
| 76 | 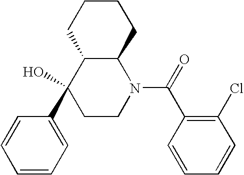 | (trans)-1-(2-chlorobenzoyl)-4- phenyldecahydroquinolin-4-ol | 370.15 |
| 77 | 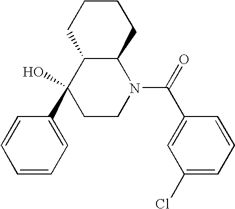 | (trans)-1-(3-chlorobenzoyl)-4- phenyldecahydroquinolin-4-ol | 302.21 |
| 78 | 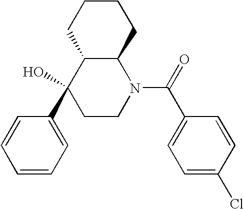 | (trans)-1-(4-chlorobenzoyl)-4- phenyldecahydroquinolin-4-ol | 370.15 |
| 79 | 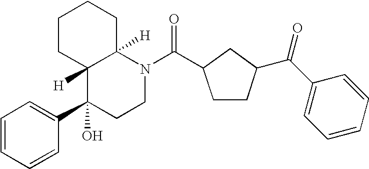 | (3-{[(trans)-4-hydroxy-4- phenyloctahydroquinolin-1(2H)- yl]carbonyl}cyclopentyl)(phenyl) methanone | 432.25 |
| 80 | 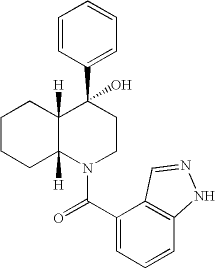 | 1-(1H-INDAZOL-4- YLCARBONYL)-4- PHENYLDECAHYDROQUINOLIN- 4-OL | 376.2005 |
| 81 | 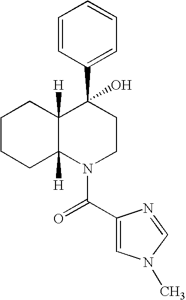 | cis-1-[(1-methyl-1H-imidazol-4- yl)carbonyl]-4- phenyldecahydroquinolin-4-ol | 340.1 |
| 82 | 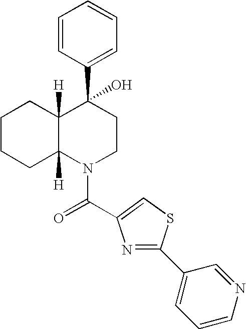 | cis-4-phenyl-1-[(2-pyridin-3-yl- 1,3-thiazol-4- yl)carbonyl]decahydroquinolin-4- ol | 420.2 |
| 83 | 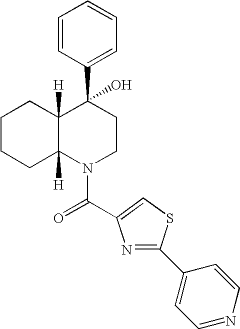 | cis-4-phenyl-1-[(2-pyridin-4-yl- 1,3-thiazol-4- yl)carbonyl]decahydroquinolin-4- ol | 420.2 |
| 84 | 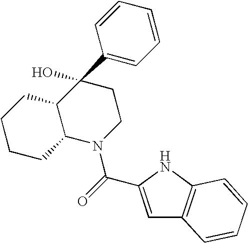 | 1-(1H-indol-2-ylcarbonyl)-4- phenyldecahydroquinolin-4-ol | 375.2055 |
| 85 | 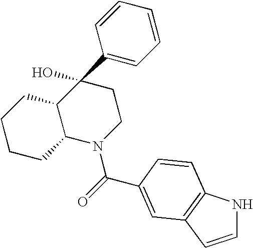 | 1-(1H-indol-5-ylcarbonyl)-4- phenyldecahydroquinolin-4-ol | 375.2054 |
| 86 | 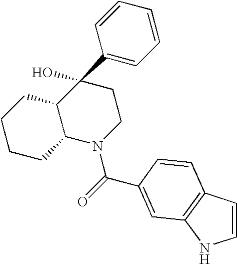 | 1-(1H-indol-6-ylcarbonyl)-4- phenyldecahydroquinolin-4-ol | 375.2057 |
| 87 | 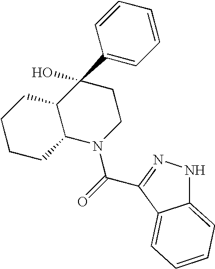 | 1-(1H-indazol-3-ylcarbonyl)-4- phenyldecahydroquinolin-4-ol | 376.2008 |
| 88 | 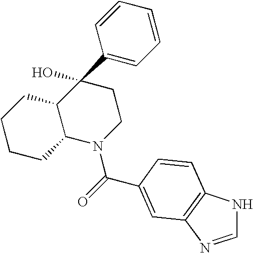 | 1-(1H-benzimidazol-5- ylcarbonyl)-4- phenyldecahydroquinolin-4-ol | 376.2012 |
| 89 | 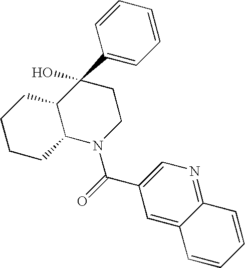 | 4-phenyl-1-(quinolin-3- ylcarbonyl)decahydroquinolin-4-ol | 387.2059 |
| 90 | 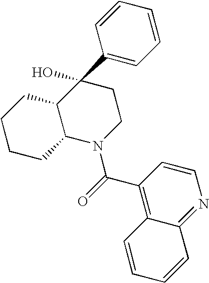 | 4-phenyl-1-(quinolin-4- ylcarbonyl)decahydroquinolin-4-ol | 387.2057 |
| 91 | 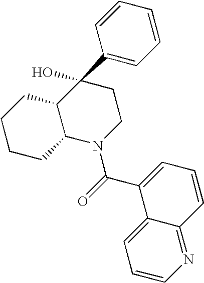 | 4-phenyl-1-(quinolin-5- ylcarbonyl)decahydroquinolin-4-ol | 387.2057 |
| 92 | 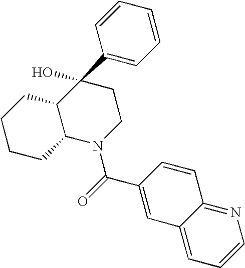 | 4-phenyl-1-(quinolin-6- ylcarbonyl)decahydroquinolin-4-ol | 387.2058 |
| 93 | 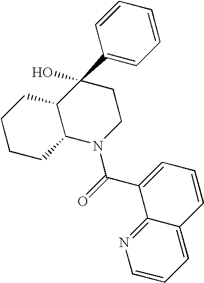 | 4-phenyl-1-(quinolin-8- ylcarbonyl)decahydroquinolin-4-ol | 387.2057 |
| 94 | 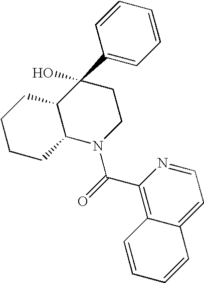 | 1-(isoquinolin-1-ylcarbonyl)-4- phenyldecahydroquinolin-4-ol | 387.2057 |
| 95 | 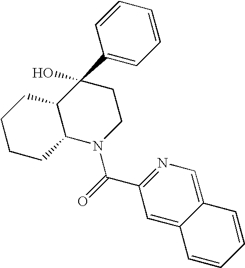 | 1-(isoquinolin-3-ylcarbonyl)-4- phenyldecahydroquinolin-4-ol | 387.2059 |
| 96 | 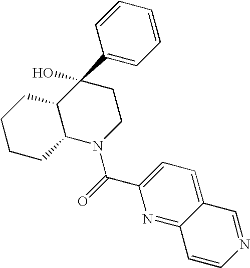 | 1-(1,6-naphthyridin-2-ylcarbonyl)- 4-phenyldecahydroquinolin-4-ol | 388.2014 |
| 97 | 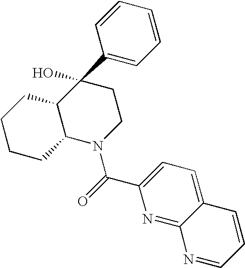 | 1-(1,8-naphthyridin-2-ylcarbonyl)- 4-phenyldecahydroquinolin-4-ol | 388.2013 |
| 98 | 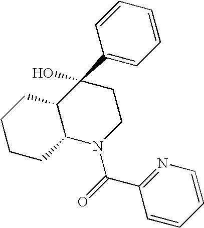 | 4-phenyl-1-(pyridin-2- ylcarbonyl)decahydroquinolin-4-ol | 337.1903 |
| 99 | 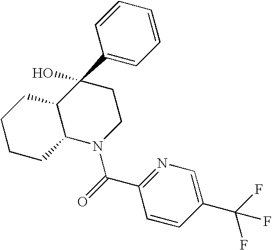 | 4-phenyl-1-{[5- (trifluoromethyl)pyridin-2- yl]carbonyl}decahydroquinolin-4- ol | 405.1773 |
| 100 | 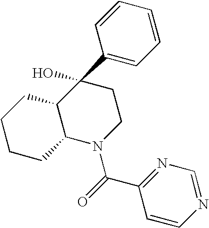 | 4-phenyl-1-(pyrimidin-4- ylcarbonyl)decahydroquinolin-4-ol | 338.1853 |
| 101 | 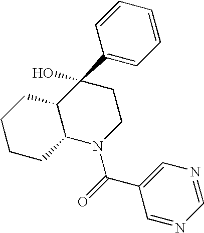 | 4-phenyl-1-(pyrimidin-5- ylcarbonyl)decahydroquinolin-4-ol | 338.1854 |
| 102 | 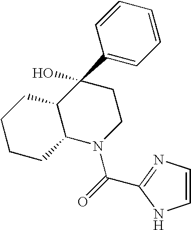 | 1-(1H-imidazol-2-ylcarbonyl)-4- phenyldecahydroquinolin-4-ol | 326.1858 |
| 103 | 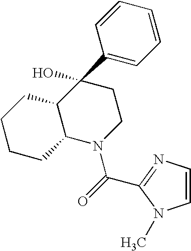 | 1-[(1-methyl-1H-imidazol-2- yl)carbonyl]-4- phenyldecahydroquinolin-4-ol | 340.2013 |
| 104 | 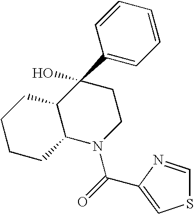 | 4-phenyl-1-(1,3-thiazol-4- ylcarbonyl)decahydroquinolin-4-ol | 343.1472 |
| 105 | 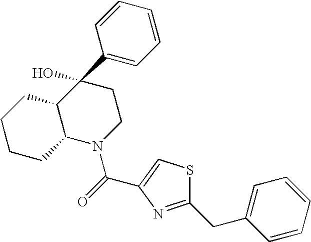 | 1-[(2-benzyl-1,3-thiazol-4- yl)carbonyl]-4- phenyldecahydroquinolin-4-ol | 433.1933 |
| 106 | 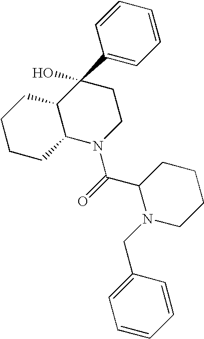 | 23. (4R,4aS,8aR)-1-[(1- benzylpiperidin-2-yl)carbonyl]-4- phenyldecahydroquinolin-4-ol | 433.2837 |
| 107 | 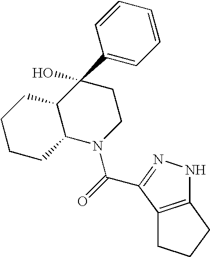 | 4-phenyl-1-(1,4,5,6- tetrahydrocyclopenta[c]pyrazol-3- ylcarbonyl)decahydroquinolin-4-ol | 366.2173 |
| 108 | 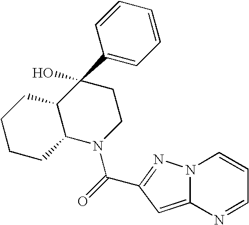 | 4-phenyl-1-(pyrazolo[1,5- a]pyrimidin-2- ylcarbonyl)decahydroquinolin-4-ol | 377.1962 |
| 109 | 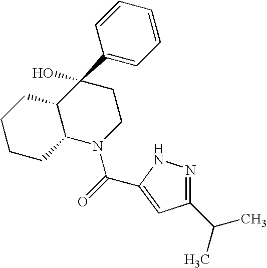 | 1-[(3-isopropyl-1H-pyrazol-5- yl)carbonyl]-4- phenyldecahydroquinolin-4-ol | 368.233 |
| 110 | 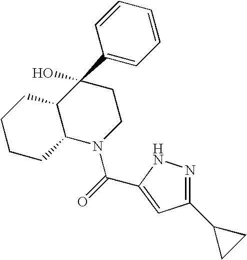 | 1-[(3-cyclopropyl-1H-pyrazol-5- yl)carbonyl]-4- phenyldecahydroquinolin-4-ol | 366.2174 |
| 111 | 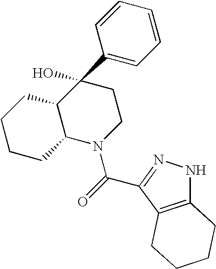 | 4-phenyl-1-(4,5,6,7-tetrahydro-1H- indazol-3- ylcarbonyl)decahydroquinolin-4-ol | 380.2328 |
| 112 | 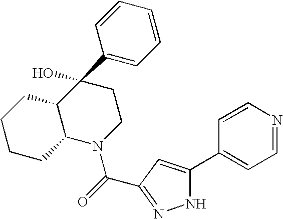 | 4-phenyl-1-[(5-pyridin-4-yl-1H- pyrazol-3- yl)carbonyl]decahydroquinolin-4- ol | 403.2126 |
| 113 | 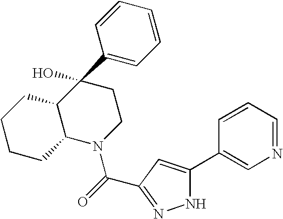 | 4-phenyl-1-[(5-pyridin-3-yl-1H- pyrazol-3- yl)carbonyl]decahydroquinolin-4- ol | 403.2123 |
| 114 | 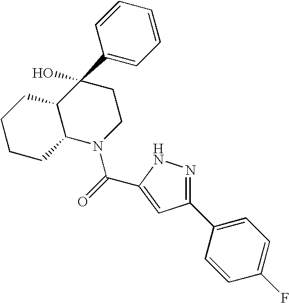 | 1-{[3-(4-fluorophenyl)-1H- pyrazol-5-yl]carbonyl}-4- phenyldecahydroquinolin-4-ol | 420.2077 |
| 115 | 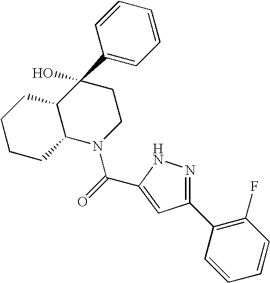 | 1-{[3-(2-fluorophenyl)-1H- pyrazol-5-yl]carbonyl}-4- phenyldecahydroquinolin-4-ol | 420.2073 |
| 116 | 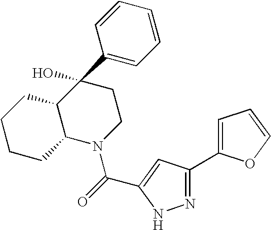 | 1-{[3-(2-furyl)-1H-pyrazol-5- yl]carbonyl}-4- phenyldecahydroquinolin-4-ol | 392.1967 |
| 117 | 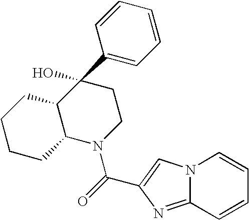 | 1-(imidazo[1,2-a]pyridin-2- ylcarbonyl)-4- phenyldecahydroquinolin-4-ol | 376.2017 |
| 118 | 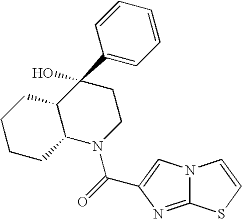 | 1-(imidazo[2,1-b][1,3]thiazol-6- ylcarbonyl)-4- phenyldecahydroquinolin-4-ol | 382.1583 |
| 119 | 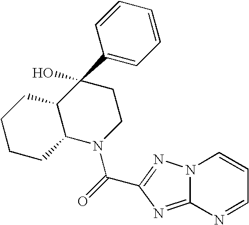 | 4-phenyl-1-([1,2,4]triazolo[1,50 a]pyrimidin-2- ylcarbonyl)decahydroquinolin-4-ol | 378.1925 |
| 120 | 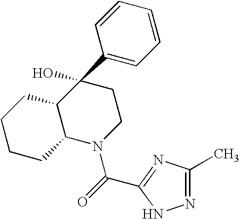 | 1-[(3-methyl-1H-1,2,4-triazol-5- yl)carbonyl]-4- phenyldecahydroquinolin-4-ol | 341.1967 |
| 121 | 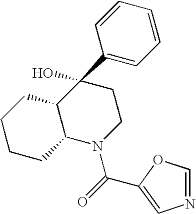 | 1-(1,3-oxazol-5-ylcarbonyl)-4- phenyldecahydroquinolin-4-ol | 327.1698 |
| 122 | 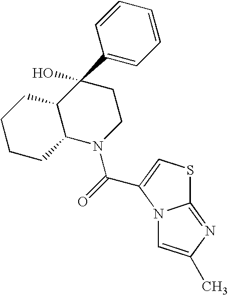 | 1-[(6-methylimidazo[2,1- b][1,3]thiazol-3-yl)carbonyl]-4- phenyldecahydroquinolin-4-ol | 396.1733 |
| 123 | 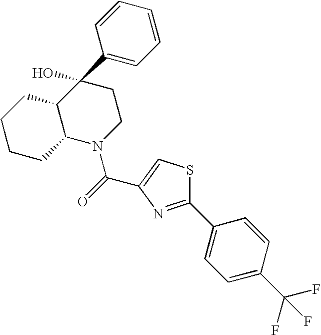 | 4-phenyl-1-({2-[4- (trifluoromethyl)phenyl]-1,3- thiazol-4- yl}carbonyl)decahydroquinolin-4- ol | 487.1647 |
| 124 | 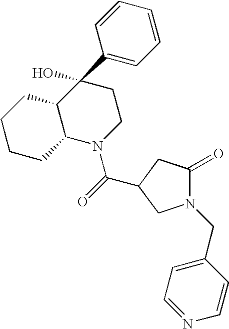 | 4-{[4-hydroxy-4- phenyloctahydroquinolin-1(2H)- yl]carbonyl}-1-(pyridin-4- ylmethyl)pyrrolidin-2-one | 434.2435 |
| 125 | 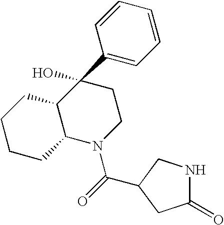 | 4-{[4-hydroxy-4- phenyloctahydroquinolin-1(2H)- yl]carbonyl}pyrrolidin-2-one | 343.201 |
| 126 | 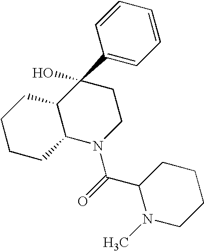 | 1-[(1-methylpiperidin-2- yl)carbonyl]-4- phenyldecahydroquinolin-4-ol | 357.2529 |
| 127 | 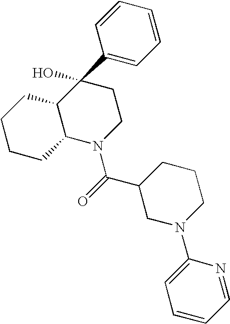 | 4-phenyl-1-[(1-pyridin-2- ylpiperidin-3- yl)carbonyl]decahydroquinolin-4- ol | 420.2636 |
| 128 | 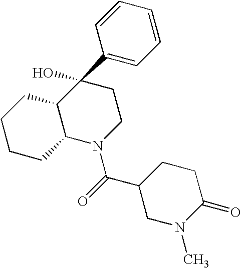 | 5-{[4-hydroxy-4- phenyloctahydroquinolin-1(2H)- yl]carbonyl}-1-methylpiperidin-2- one | 371.2326 |
| 129 | 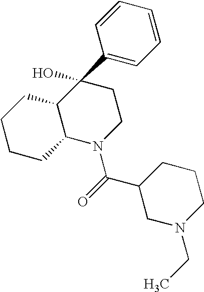 | 1-[(1-ethylpiperidin-3- yl)carbonyl]-4- phenyldecahydroquinolin-4-ol | 371.2684 |
| 130 | 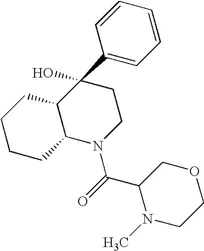 | 1-[(4-methylmorpholin-3- yl)carbonyl]-4- phenyldecahydroquinolin-4-ol | 359.2322 |
| 131 | 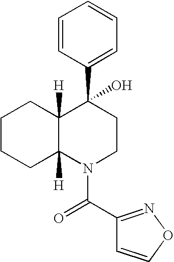 | 1-(isoxazol-3-ylcarbonyl)-4- phenyldecahydroquinolin-4-ol (Enantiomer B) | 327.1729 |
| 132 | 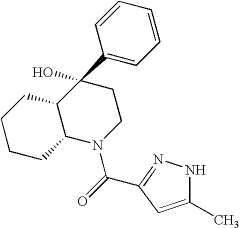 | cis-1-[(5-methyl-1H-pyrazol-3- yl)carbonyl]-4- phenyldecahydroquinolin-4-ol | 340.2009 |
| 133 | 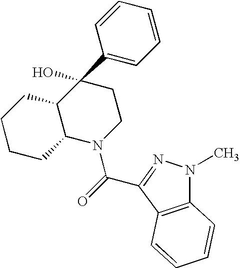 | cis-1-[(1-methyl-1H-indazol-3- yl)carbonyl]-4- phenyldecahydroquinolin-4-ol | 390.2163 |
| 134 | 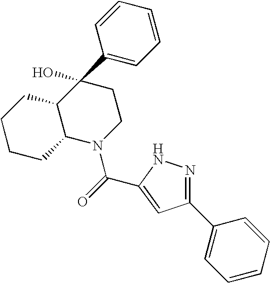 | cis-4-phenyl-1-[(3-phenyl-1H- pyrazol-5- yl)carbonyl]decahydroquinolin-4- ol | 402.2165 |
| 135 | 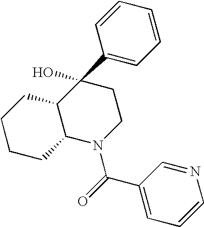 | cis-4-phenyl-1-(pyridin-3- ylcarbonyl)decahydroquinolin-4-ol | 337.1903 |
| 136 | 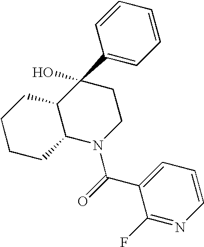 | cis-1-[(2-fluoropyridin-3- yl)carbonyl]-4- phenyldecahydroquinolin-4-ol | 355.1806 |
| 137 | 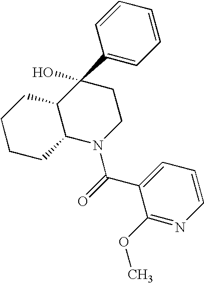 | cis-1-[(2-methoxypyridin-3- yl)carbonyl]-4- phenyldecahydroquinolin-4-ol | 367.2007 |
| 138 | 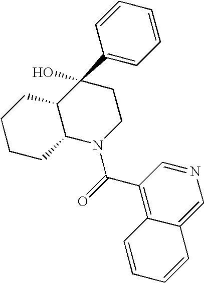 | cis-1-(isoquinolin-4-ylcarbonyl)-4- phenyldecahydroquinolin-4-ol | 387.2058 |
| 139 | 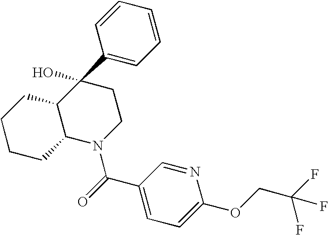 | cis-4-phenyl-1-{[6-(2,2,2- trifluoroethoxy)pyridin-3- yl]carbonyl}decahydroquinolin-4- ol | 435.1878 |
| 140 | 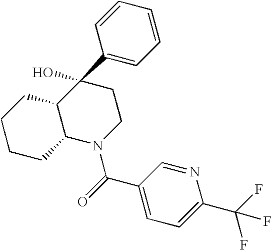 | cis-4-phenyl-1-{[6- (trifluoromethyl)pyridin-3- yl]carbonyl}decahydroquinolin-4- ol | 405.177 |
| 141 | 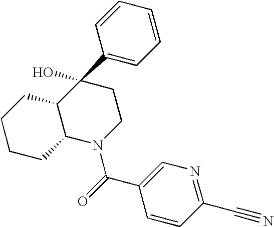 | 5-cis-4-hydroxy-4- phenyloctahydroquinolin-1(2H)- yl]carbonyl}pyridine-2- carbonitrile | 362.1853 |
| 142 | 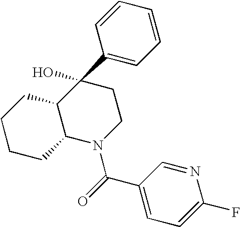 | cis-1-[(6-fluoropyridin-3- yl)carbonyl]-4- phenyldecahydroquinolin-4-ol | 355.1807 |
| 143 | 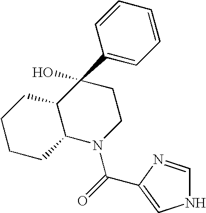 | cis-1-(1H-imidazol-4-ylcarbonyl)- 4-phenyldecahydroquinolin-4-ol | 326.1857 |
| 144 | 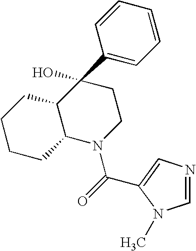 | cis-1-[(1-methyl-1H-imidazol-5- yl)carbonyl]-4- phenyldecahydroquinolin-4-ol | 340.2011 |
| 145 | 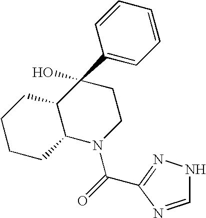 | cis-4-phenyl-1-(1H-1,2,4-triazol-3- ylcarbonyl)decahydroquinolin-4-ol | 327.1809 |
| 146 | 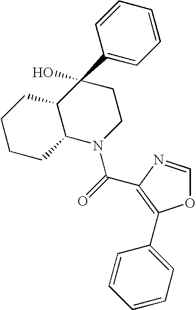 | cis-4-phenyl-1-[(5-phenyl-1,3- oxazol-4- yl)carbonyl]decahydroquinolin-4- ol | 403.2001 |
| 147 | 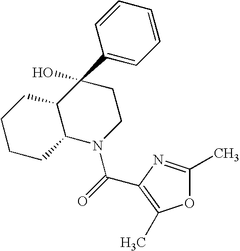 | cis-1-[(2,5-dimethyl-1,3-oxazol-4- yl)carbonyl]-4- phenyldecahydroquinolin-4-ol | 355.2006 |
| 148 | 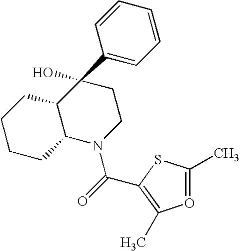 | cis-1-[(2,4-dimethyl-1,3-thiazol-5- yl)carbonyl]-4- phenyldecahydroquinolin-4-ol | 371.178 |
| 149 | 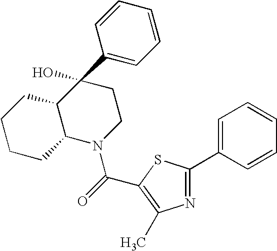 | cis-1-[(4-methyl-2-phenyl-1,3- thiazol-5-yl)carbonyl]-4- phenyldecahydroquinolin-4-ol | 433.1928 |
| 150 | 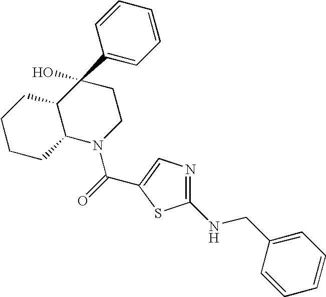 | cis-1-{[2-(benzylamino)-1,3- thiazol-5-yl]carbonyl}-4- phenyldecahydroquinolin-4-ol | 448.2039 |
| 151 | 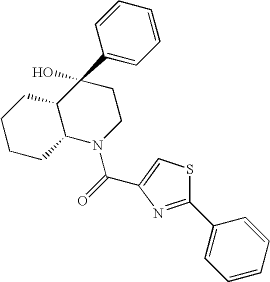 | cis-4-phenyl-1-[(2-phenyl-1,3- thiazol-4- yl)carbonyl]decahydroquinolin-4- ol | 419.1773 |
| 152 | 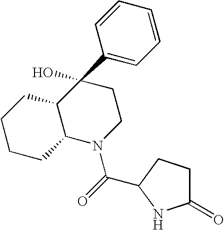 | 5-{[cis-4-hydroxy-4- phenyloctahydroquinolin-1(2H)- yl]carbonyl}pyrrolidin-2-one | 343.2009 |
| 153 | 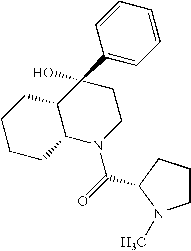 | cis-1-(1-methyl-L-prolyl)-4- phenyldecahydroquinolin-4-ol | 343.237 |
| 154 | 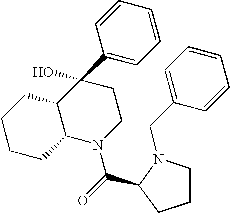 | cis-1-(1-benzyl-L-prolyl)-4- phenyldecahydroquinolin-4-ol | 419.2675 |
| 155 | 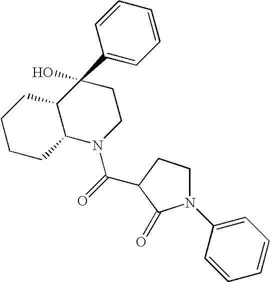 | 3-{[cis-4-hydroxy-4- phenyloctahydroquinolin-1(2H)- yl]carbonyl}-1-phenylpyrrolidin-2- one | 419.2675 |
| 156 | 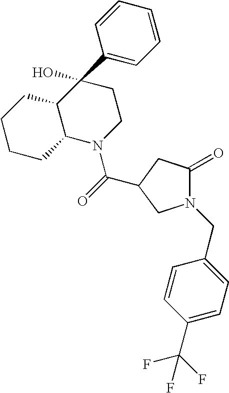 | 4-{[cis-4-hydroxy-4- phenyloctahydroquinolin-1(2H)- yl]carbonyl}-1-[4- (trifluoromethyl)benzyl]pyrrolidin- 2-one | 501.234 |
| 157 | 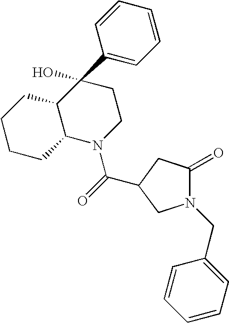 | 1-benzyl-4-{[cis-4-hydroxy-4- phenyloctahydroquinolin-1(2H)- yl]carbonyl}pyrrolidin-2-one | 433.2466 |
| 158 | 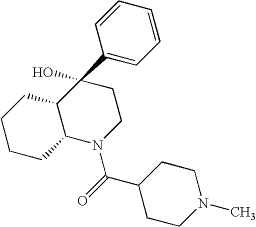 | cis-1-[(1-methylpiperidin-4- yl)carbonyl]-4- phenyldecahydroquinolin-4-ol | 357.253 |
| 159 | 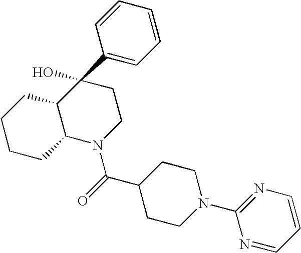 | cis-4-phenyl-1-[(1-pyrimidin-2- ylpiperidin-4- yl)carbonyl]decahydroquinolin-4- ol | 421.2591 |
| 160 | 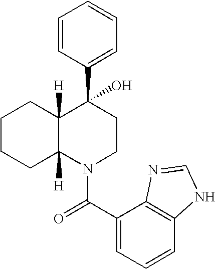 | 1-(1H-benzimidazol-4- ylcarbonyl)-4- phenyldecahydroquinolin-4-ol | 376.1986 |
| 161 | 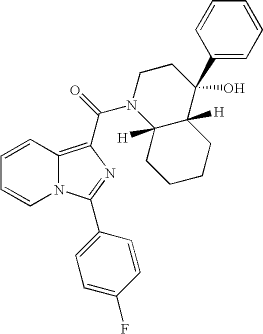 | (cis)-1-{[3-(4- fluorophenyl)imidazo[1,5- a]pyridin-1-yl]carbonyl}-4- phenyldecahydroquinolin-4-ol | 470.2226 |
| 162 | 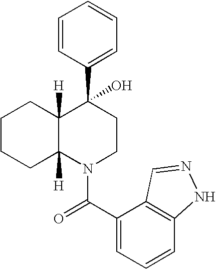 | 1-(1H-indazol-4-ylcarbonyl)-4- phenyldecahydroquinolin-4-ol (Enantiomer A) | 376.1991 |
| 163 | 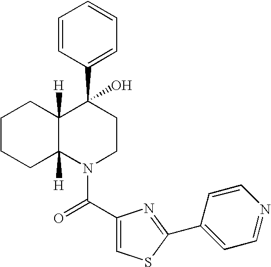 | 4-phenyl-1-[(2-pyridin-4-yl-1,3- thiazol-4- yl)carbonyl]decahydroquinolin-4- ol (Enantiomer A) | 420.1717 |
| 164 | 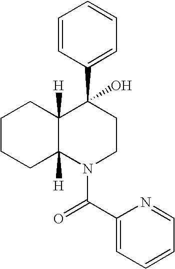 | 4-phenyl-1-(pyridin-2- ylcarbonyl)decahydroquinolin-4-ol (Enantiomer A) | 337.1869 |
| 165 | 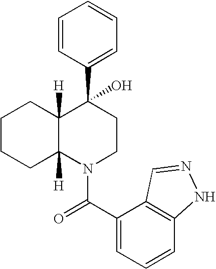 | 1-(1H-indazol-4-ylcarbonyl)-4- phenyldecahydroquinolin-4-ol (Enantiomer B) | 376.1992 |
| 166 | 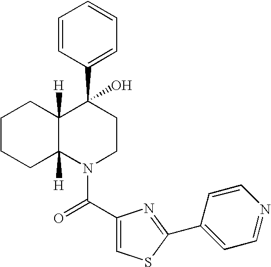 | 4-phenyl-1-[(2-pyridin-4-yl-1,3- thiazol-4- yl)carbonyl]decahydroquinolin-4- ol (Enantiomer B) | 420.1717 |
| 167 | 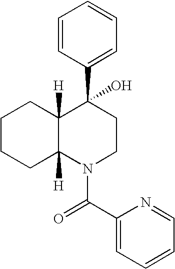 | 4-phenyl-1-(pyridin-2- ylcarbonyl)decahydroquinolin-4-ol (Enantiomer B) | 337.1874 |
| 168 | 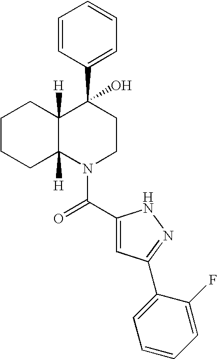 | 1-{[3-(2-fluorophenyl)-1h-pyrazol- 5-yl]carbonyl}-4- phenyldecahydroquinolin-4-ol (Enantiomer B) | 420.2058 |
| 169 | 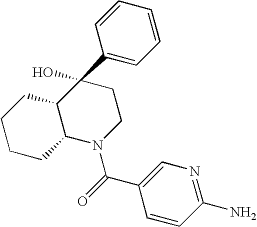 | 1-[(6-aminopyridin-3-yl)carbonyl]- 4-phenyldecahydroquinolin-4-ol | 352.2013 |
| 170 | 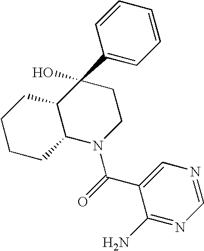 | 1-[(4-aminopyrimidin-5- yl)carbonyl]-4- phenyldecahydroquinolin-4-ol | 353.1958 |
| 171 | 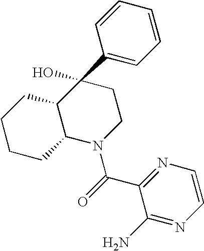 | 1-[(3-aminopyrazin-2- yl)carbonyl]-4- phenyldecahydroquinolin-4-ol | 353.1962 |
| 172 | 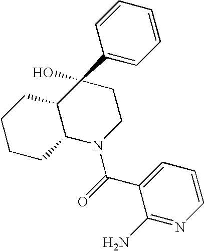 | 1-[(2-aminopyridin-3-yl)carbonyl]- 4-phenyldecahydroquinolin-4-ol | 352.2006 |
| 173 | 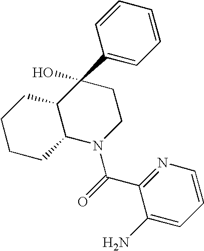 | 1-[(3-aminopyridin-2-yl)carbonyl]- 4-phenyldecahydroquinolin-4-ol | 352.2006 |
| 174 | 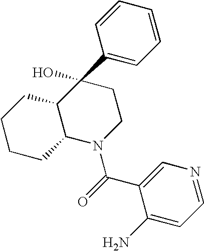 | 1-[(4-aminopyridin-3-yl)carbonyl]- 4-phenyldecahydroquinolin-4-ol | 352.2006 |
| 175 | 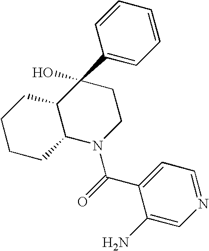 | 1-(3-aminoisonicotinoyl)-4- phenyldecahydroquinolin-4-ol | 352.2006 |
| 176 | 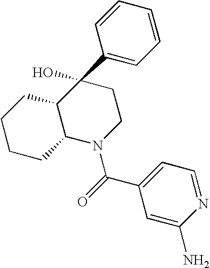 | 1-(2-aminoisonicotinoyl)-4- phenyldecahydroquinolin-4-ol | 352.2008 |
| 177 | 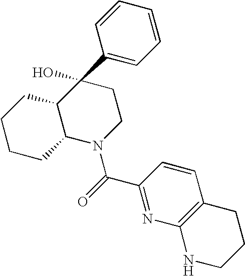 | 4-phenyl-1-(5,6,7,8-tetrahydro- 1,8-naphthyridin-2- ylcarbonyl)decahydroquinolin-4-ol | 392.2316 |
| 178 | 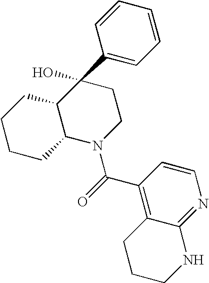 | 4-phenyl-1-(5,6,7,8-tetrahydro- 1,8-naphthyridin-4- ylcarbonyl)decahydroquinolin-4-ol | 392.2318 |
| 179 | 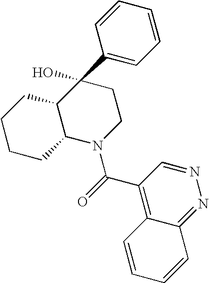 | 1-(cinnolin-4-ylcarbonyl)-4- phenyldecahydroquinolin-4-ol | 388.2005 |
| 180 | 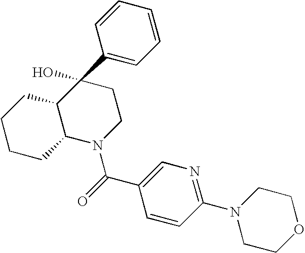 | 1-[(6-morpholin-4-ylpyridin-3- yl)carbonyl]-4- phenyldecahydroquinolin-4-ol | 422.242 |
| 181 | 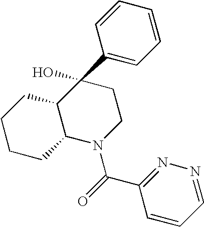 | 4-phenyl-1-(pyridazin-3- ylcarbonyl)decahydroquinolin-4-ol | 338.1851 |
| 182 | 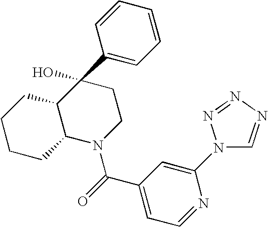 | 4-phenyl-1-[2-(1H-tetrazol-1- yl)isonicotinoyl]decahydroquinolin- 4-ol | 405.2016 |
| 183 | 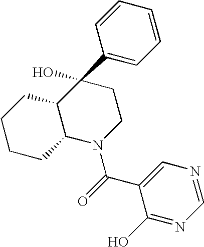 | 1-[(4-hydroxypyrimidin-5- yl)carbonyl]-4- phenyldecahydroquinolin-4-ol | 354.1801 |
| 184 | 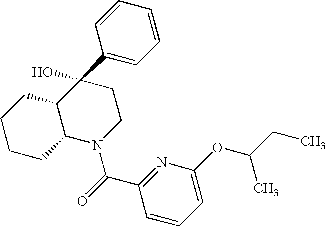 | 1-[(6-sec-butoxypyridin-2- yl)carbonyl]-4- phenyldecahydroquinolin-4-ol | 409.247 |
| 185 | 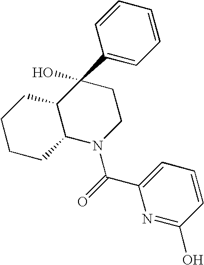 | 1-[(6-hydroxypyridin-2- yl)carbonyl]-4- phenyldecahydroquinolin-4-ol | 353.1847 |
| 186 | 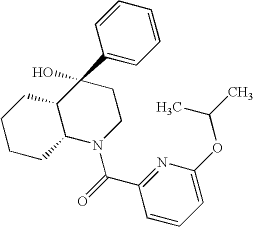 | 1-[(6-isopropoxypyridin-2- yl)carbonyl]-4- phenyldecahydroquinolin-4-ol | 395.2312 |
| 187 | 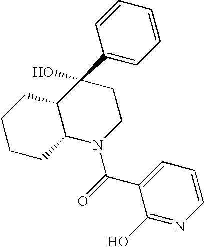 | 1-[(2-hydroxypyridin-3- yl)carbonyl]-4- phenyldecahydroquinolin-4-ol | 353.1846 |
| 188 | 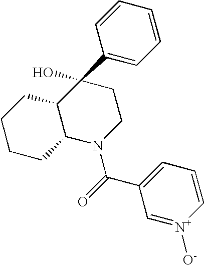 | 1-[(1-oxidopyridin-3-yl)carbonyl]- 4-phenyldecahydroquinolin-4-ol | 353.1846 |
| 189 | 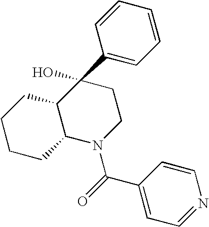 | 1-isonicotinoyl-4- phenyldecahydroquinolin-4-ol | 337.1898 |
| 190 | 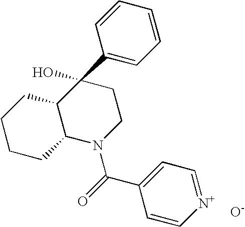 | 1-(1-oxidoisonicotinoyl)-4- phenyldecahydroquinolin-4-ol | 353.1847 |
| 191 | 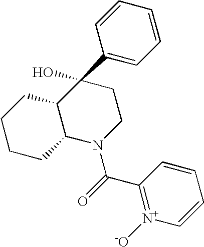 | 1-[(1-oxidopyridin-2-yl)carbonyl]- 4-phenyldecahydroquinolin-4-ol | 353.1847 |
| 192 | 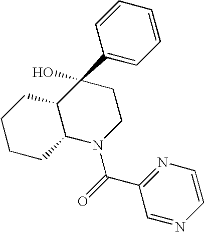 | cis-4-phenyl-1-(pyrazin-2- ylcarbonyl)decahydroquinolin-4-ol | 338.1859 |
| 193 | 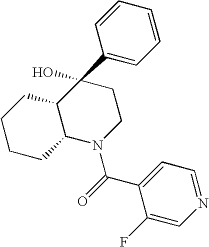 | cis-1-(3-fluoroisonicotinoyl)-4- phenyldecahydroquinolin-4-ol | 355.1808 |
| 194 | 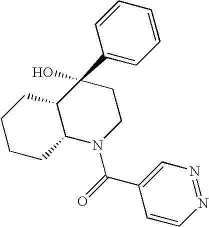 | cis-4-phenyl-1-(pyridazin-4- ylcarbonyl)decahydroquinolin-4-ol | 338.1859 |
| 195 | 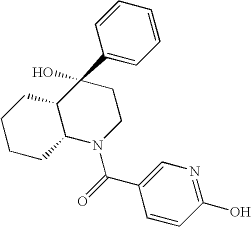 | cis-1-[(6-hydroxypyridin-3- yl)carbonyl]-4- phenyldecahydroquinolin-4-ol | 353.1862 |
| 196 | 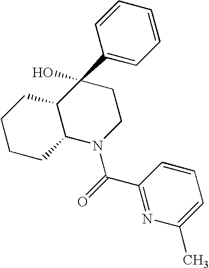 | cis-1-[(6-methylpyridin-2- yl)carbonyl]-4- phenyldecahydroquinolin-4-ol | 351.2055 |
| 197 | 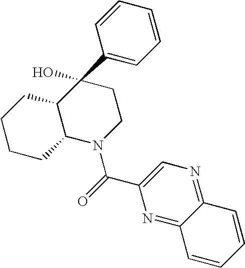 | cis-4-phenyl-1-(quinoxalin-2- ylcarbonyl)decahydrogurnolin-4-ol | 388.2014 |
| 198 | 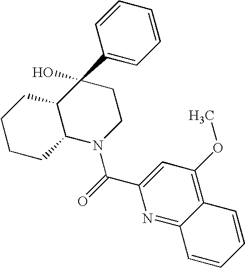 | cis-1-[(4-methoxyquinolin-2- yl)carbonyl]-4- phenyldecahydroquinolin-4-ol | 417.2158 |
| 199 | 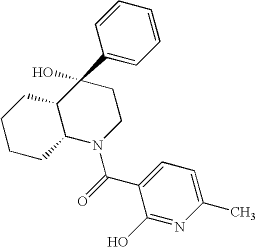 | cis-1-[(2-hydroxy-6- methylpyridin-3-yl)carbonyl]-4- phenyldecahydroquinolin-4-ol | 367.2021 |
| 200 | 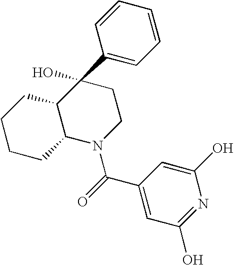 | 4-{[cis-4-hydroxy-4- phenyloctahydroquinolin-1(2H)- yl]carbonyl}pyridine-2,6-diol | 369.1812 |
| 201 | 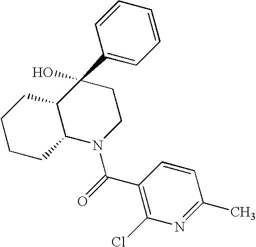 | cis-1-[(2-chloro-6-methylpyridin- 3-yl)carbonyl]-4- phenyldecahydroquinolin-4-ol | 385.1675 |
| 202 | 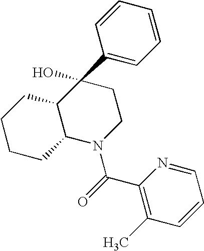 | cis-1-[(3-methylpyridin-2- yl)carbonyl]-4- phenyldecahydroquinolin-4-ol | 351.206 |
| 203 | 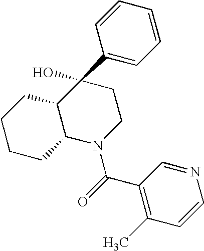 | cis-1-[(4-methylpyridin-3- yl)carbonyl]-4- phenyldecahydroquinolin-4-ol | 351.2058 |
| 204 | 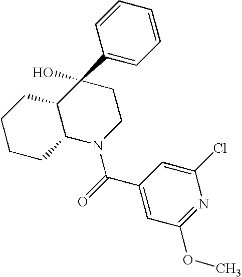 | cis-1-(2-chloro-6- methoxylsonicotinoyl)-4- phenyldecahydroquinolin-4-ol | 401.1616 |
| 205 | 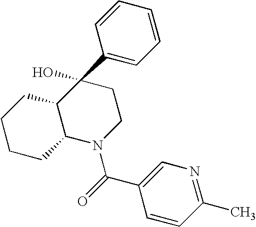 | cis)-1-[(6-methylpyridin-3- yl)carbonyl]-4- phenyldecahydroquinolin-4-ol | 351.2059 |
| 206 | 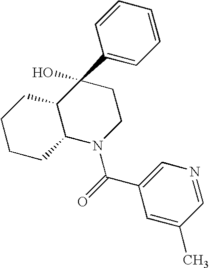 | cis-1-[(5-methylpyridin-3- yl)carbonyl]-4- phenyldecahydroquinolin-4-ol | 351.2058 |
| 207 | 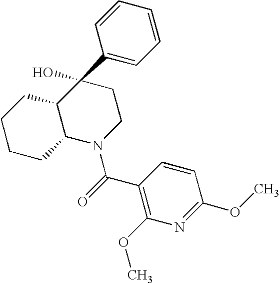 | cis-1-[(2,6-dimethoxypyridin-3- yl)carbonyl]-4- phenyldecahydroquinolin-4-ol | 397.2117 |
| 208 | 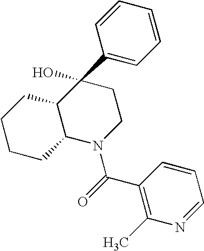 | cis-1-[(2-methylpyridin-3- yl)carbonyl]-4- phenyldecahydroquinolin-4-ol | 351.2058 |
| 209 | 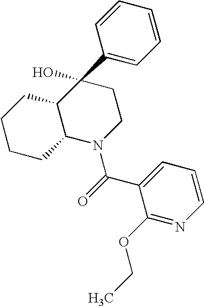 | cis-1-[(2-ethoxypyridin-3- yl)carbonyl]-4- phenyldecahydroquinolin-4-ol | 381.2165 |
| 210 | 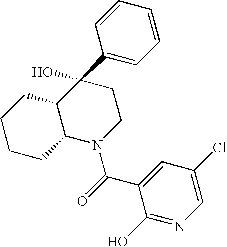 | cis-1-[(5-chloro-2-hydroxypyridin- 3-yl)carbonyl]-4- phenyldecahydroquinolin-4-ol | 387.1478 |
| 211 | 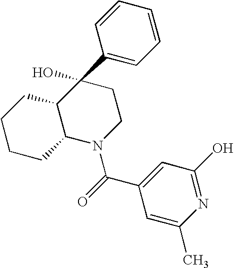 | cis-(2-hydroxy-6- methylisonicotinoyl)-4- phenyldecahydroquinolin-4-ol | 367.2022 |
| 212 | 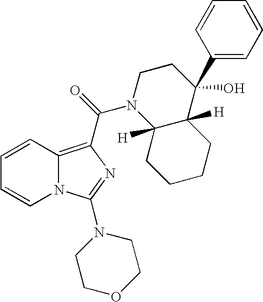 | (cis)-1-[(3-morpholin-4- ylimidazo[1,5-a]pyridin-1- yl)carbonyl]-4- phenyldecahydroquinolin-4-ol | 461.2531 |
| 213 | 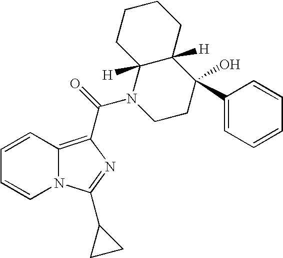 | (Cis)-1-[(3- cyclopropylimidazo[1,5-a]pyridin- 1-yl)carbonyl]-4- phenyldecahydroquinolin-4-oL | 416.2337 |
| 214 | 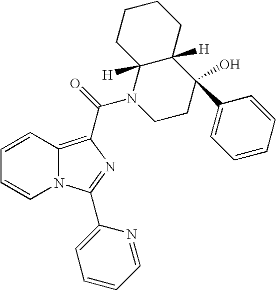 | (Cis)-4-phenyl-1-[(3-pyridin-2- ylimidazo[1,5-a]pyridin-1- yl)carbonyl]decahydroquinolin-4- ol | 453.2276 |
| 215 | 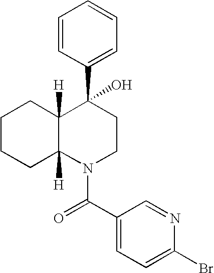 | cis-1-[(6-bromopyridin-3- yl)carbonyl]-4- phenyldecahydroquinolin-4-ol | 416.1 |
| 216 | 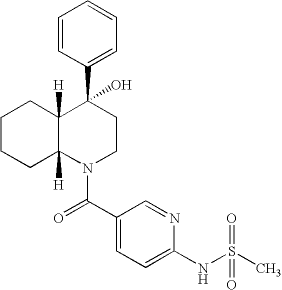 | N-(5-{[cis-4-hydroxy-4- phenyloctahydroquinolin-1(2H)- yl]carbonyl}pyridin-2- yl)methanesulfonamide | 430.2 |
| 217 | 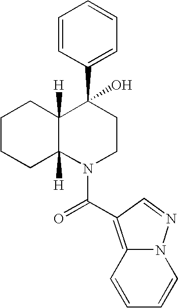 | 4-phenyl-1-(pyrazolo[1,5- a]pyridin-3- ylcarbonyl)decahydroquinolin-4-ol | 376.2 |
| 218 | 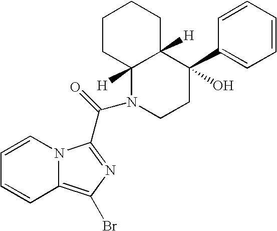 | 1-[(1-bromoimidazo[1,5-a]pyridin- 3-yl)carbonyl]-4- phenyldecahydroquinolin-4-ol | 454.1, 456.1 |
| 219 | 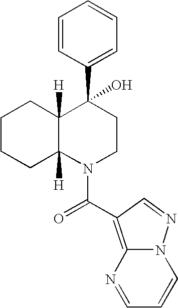 | 4-phenyl-1-(pyrazolo[1,5- a]pyrimidin-3- ylcarbonyl)decahydroquinolin-4-ol | 377.1 |
| 220 | 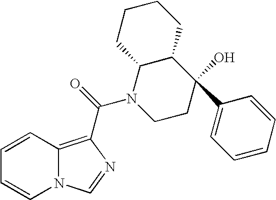 | (cis)-1-(imidazo[1,5-a]pyridin-1- ylcarbonyl)-4- phenyldecahydroquinolin-4-ol | 376.202 |
| 221 | 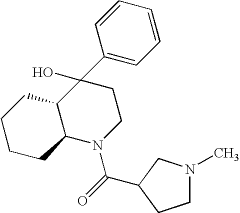 | cis-1-[(1-methylpyrrolidin-3- yl)carbonyl]-4- phenyldecahydroquinolin-4-ol | 343.2383 |
| 222 | 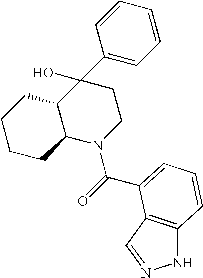 | cis-1-(1H-indazol-4-ylcarbonyl)-4- phenyldecahydroquinolin-4-ol | 376.2029 |
| 223 | 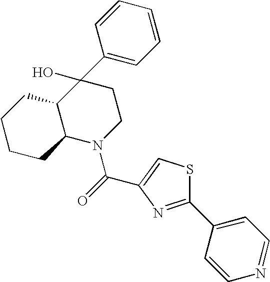 | cis-4-phenyl-1-[(2-pyridin-4-yl- 1,3-thiazol-4- yl)carbonyl]decahydroquinolin-4- ol | 420.1733 |
| 224 | 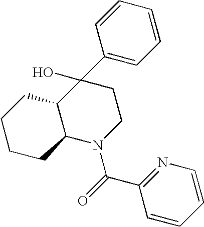 | cis-4-phenyl-1-(pyridin-2- ylcarbonyl)decahydroquinolin-4-ol | 337.1918 |
| 225 | 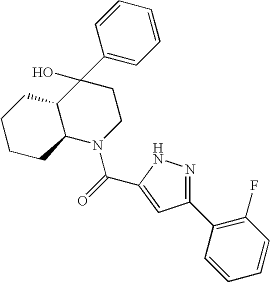 | cis-1-{[3-(2-fluorophenyl)-1H- pyrazol-5-yl]carbonyl}-4- phenyldecahydroquinohn-4-ol | 420.208 |
| 226 | 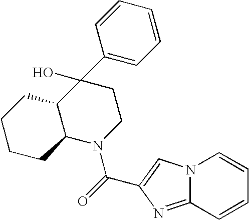 | cis-1-(imidazo[1,2-a]pyridin-2- ylcarbonyl)-4- phenyldecahydroquinolin-4-ol | 376.2021 |
| 227 | 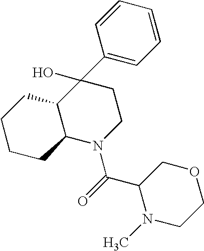 | cis-1-[(4-methylmorpholin-3- yl)carbonyl]-4- phenyldecahydroquinolin-4-ol | 359.2329 |
| 228 | 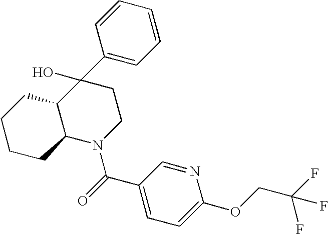 | cis-4-phenyl-1-{[6-(2,2,2- trifluoroethoxy)pyridin-3- yl]carbonyl}decahydroquinolin-4- ol | 435.1884 |
| 229 | 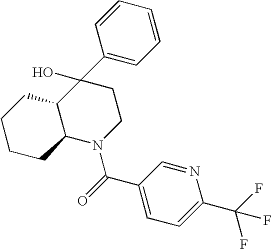 | cis-4-phenyl-1-{[6- (trifluoromethyl)pyridin-3- yl]carbonyl}decahydroquinolin-4- ol | 405.1779 |
| 230 | 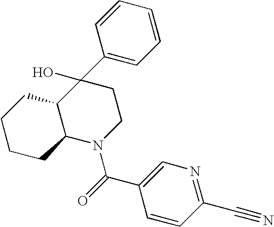 | 5-{[cis-4-hydroxy-4- phenyloctahydroquinolin-1(2H)- yl]carbonyl}pyridine-2- carbonitrile | 362.1874 |
| 231 | 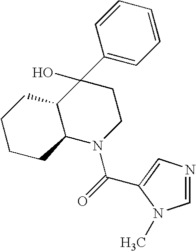 | cis-1-[(1-methyl-1H-imidazol-5- yl)carbonyl]-4- phenyldecahydroquinolin-4-ol | 340.2024 |
| 232 | 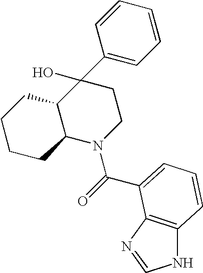 | cis-1-(1H-benzimidazol-4- ylcarbonyl)-4- phenyldecahydroquinolin-4-ol | 376.2023 |
| 233 | 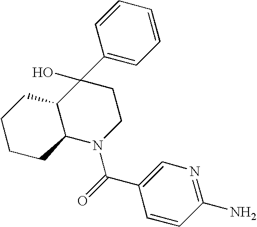 | cis-1-[(6-aminopyridin-3- yl)carbonyl]-4- phenyldecahydrogurnolin-4-ol | 352.2023 |
| 234 | 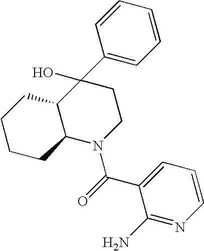 | cis-1-[(2-aminopyridin-3- yl)carbonyl]-4- phenyldecahydroquinolin-4-ol | 352.2022 |
| 235 | 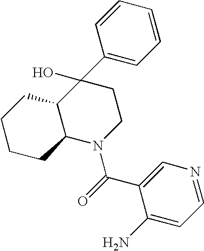 | cis-1-[(4-aminopyridin-3- yl)carbonyl]-4- phenyldecahydroquinolin-4-ol | 352.2021 |
| 236 | 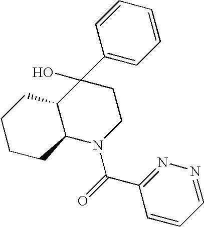 | cis-4-phenyl-1-(pyridazin-3- ylcarbonyl)decahydroquinolin-4-ol | 320.1768 |
| 237 | 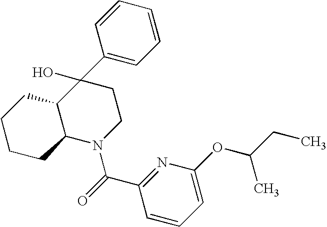 | cis-1-[(6-sec-butoxypyridin-2- yl)carbonyl]-4- phenyldecahydroquinolin-4-ol | 409.248 |
| 238 | 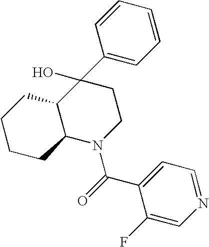 | cis-1-(3-fluoroisonicotinoyl)-4- phenyldecahydroquinolin-4-ol | 355.182 |
| 239 | 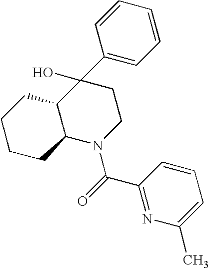 | cis-1-[(6-methylpyridin-2- yl)carbonyl]-4- phenyldecahydroquinolin-4-ol | 351.2073 |
| 240 | 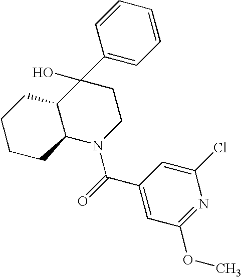 | cis-1-(2-chloro-6- methoxylsonicotinoyl)-4- phenyldecahydroquinolin-4-ol | 401.1628 |
| 241 | 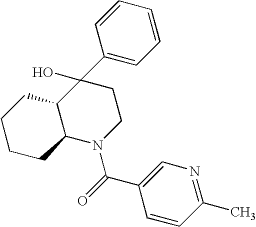 | cis-1-[(6-methylpyridin-3- yl)carbonyl]-4- phenyldecahydroquinolin-4-ol | 351.2071 |
| 242 | 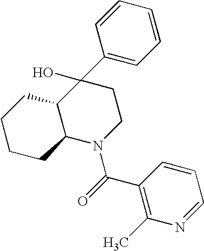 | cis-1-[(2-methylpyridin-3- yl)carbonyl]-4- phenyldecahydroquinolin-4-ol | 351.2071 |
| 243 | 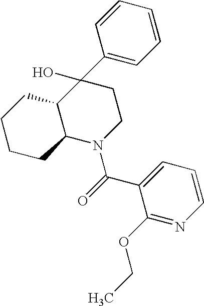 | cis-1-[(2-ethoxypyridin-3- yl)carbonyl]-4- phenyldecahydroquinolin-4-ol | 381.2172 |
| 244 | 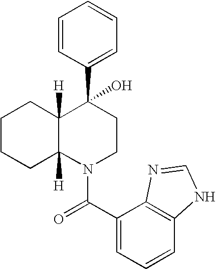 | 1-(1H-benzimidazol-4- ylcarbonyl)-4- phenyldecahydroquinolin-4-ol (Enantiomer B) | 376.2004 |
| 245 | 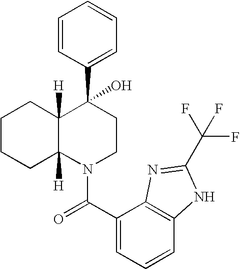 | 4-phenyl-1-{[2-(trifluoromethyl)- 1H-benzimidazol-4- yl]carbonyl}decahydroquinolin-4- ol (Enantiomer B) | 444.1884 |
| 246 | 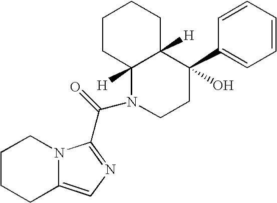 | 4-phenyl-1-(5,6,7,8- tetrahydroimidazo[1,5-a]pyridin-3- ylcarbonyl)decahydroquinolin-4-ol | 380.2 |
| 247 | 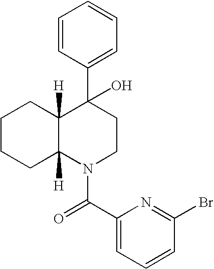 | cis-1-[(6-bromopyridin-2- yl)carbonyl]-4- phenyldecahydroquinolin-4-ol | 416.1 |
| 248 | 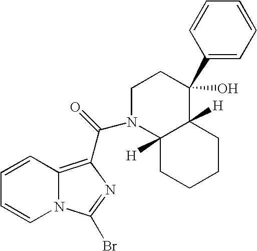 | (cis)-1-[(3-bromoimidazo[1,5- a]pyridin-1-yl)carbonyl]-4- phenyldecahydroquinolin-4-ol | 456.1087 |
| 249 | 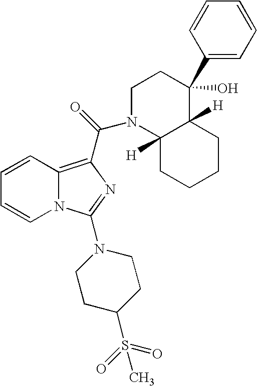 | (cis)-1-({3-[4- (methylsulfonyl)piperidin-1- yl]imidazo[1,5-a]pyridin-1- yl}carbonyl)-4- phenyldecahydroquinolin-4-ol | 537.253 |
| 250 | 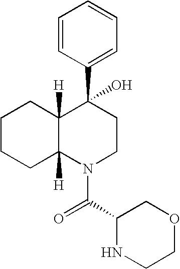 | 1-[(3S)-morpholin-3-ylcarbonyl]- 4-phenyldecahydroquinolin-4-ol (Enantiomer B) | 345.2157 |
| 251 | 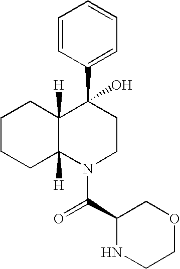 | 1-[(3R)-morpholin-3-ylcarbonyl]- 4-phenyldecahydroquinolin-4-ol (Enantiomer B) | 345.2157 |
| 252 | 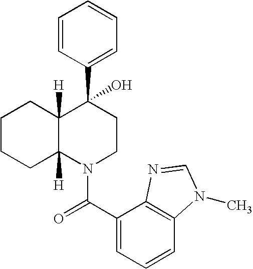 | 1-[(1-methyl-1h-benzimidazol-4- yl)carbonyl]-4- phenyldecahydroquinolin-4-ol (Enantiomer B) | 390.2163 |
| 253 | 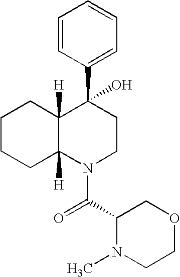 | 1-{[(3S)-4-methylmorpholin-3- yl]carbonyl}-4- phenyldecahydroquinolin-4-ol (Enantiomer B) | 359.2309 |
| 254 | 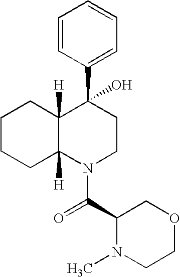 | 1-{[(3R)-4-methylmorpholin-3- yl]carbonyl}-4- phenyldecahydroquinolin-4-ol (Enantiomer B) | 359.2308 |
| 255 | 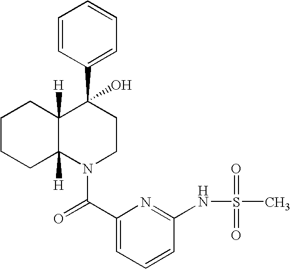 | N-(6-{[cis-4-hydroxy-4- phenyloctahydroquinolin-1(2H)- yl]carbonyl}pyridin-2- yl)methanesulfonamide | 430.2 |
| 256 | 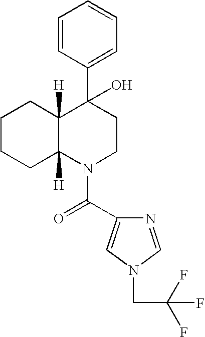 | cis-4-phenyl-1-{[1-(2,2,2- trifluoroethyl)-1H-imidazol-4- yl]carbonyl}decahydroquinolin-4- ol | 408.2 |
| 292 | 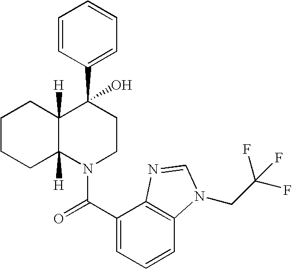 | (CIS)-4-PHENYL-1-{[1- (2,2,2- TRIFLUOROETHYL)- 1H-BENZIMIDAZOL-4- YL]CARBONYL} DECAHYDROQUINOLIN-4-OL | 458.2045 |
| 293 | 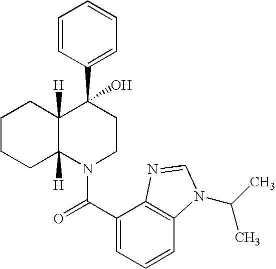 | (CIS)1-{(1- ISOPROPYL-1H- BENZIMIDAZOL-4- YL)CARBONYL]-4- PHENYLDECAHYDROQUINOLIN- 4-OL | 418.2491 |
| 294 | 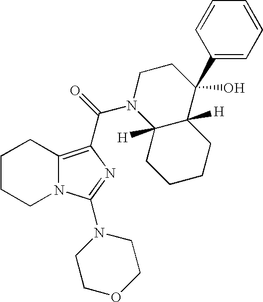 | (cis)-1-[(3-MORPHOLIN-4-YL-5,6,7,8- TETRAHYDROIMIDAZO[1,5- A]PYRIDIN-1-YL)CARBONYL]-4- PHENYLDECAHYDROQUINOLIN-4- OL | 465.2832 |
| 295 | 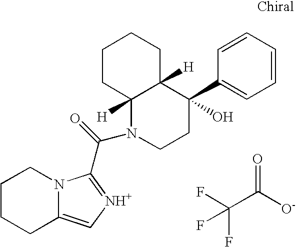 | 4-PHENYL-1-(5,6,7,8- TETRAHYDROIMIDAZO[1,5- A]PYRIDIN-3- YLCARBONYL) DECAHYDROQUINOLIN-4-OL TRIFLUOROACETATE | 380.3 |
| 296 | 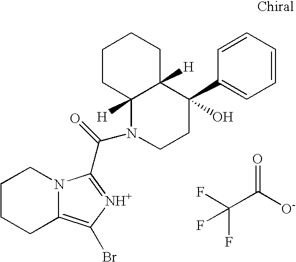 | 1-[(1-BROMO-5,6,7,8- TETRAHYDROIMIDAZO[1,5- A]PYRIDIN-3-YL)CARBONYL]-4- PHENYLDECAHYDROQUINOLIN-4- OL TRIFLUOROACETATE | 458.2, 460.2 |
| 297 | 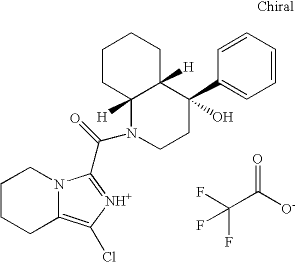 | 1-[(1-CHLORO-5,6,7,8- TETRAHYDROIMIDAZO[1,5- A]PYRIDIN-3-YL)CARBONYL]-4- PHENYLDECAHYDROQUINOLIN-4- OL TRIFLUOROACETATE | 414.3 |
| 298 | 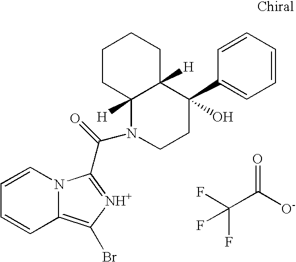 | 1-[(1-BROMOIMIDAZO[1,5- A]PYRIDIN-3-YL)CARBONYL]-4- PHENYLDECAHYDROQUINOLIN-4- OL TRIFLUOROACETATE | 454.2, 456.2 |
| 299 | 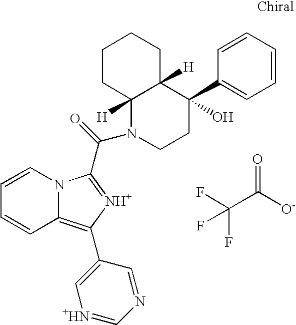 | 4-PHENYL-1-[(1-PYRIMIDIN-5- YLIMIDAZO[1,5-A]PYRIDIN-3- YL)CARBONYL] DECAHYDROQUINOLIN- 4-OL BIS- TRIFLUOROACETATE | 454.4 |
| 300 | 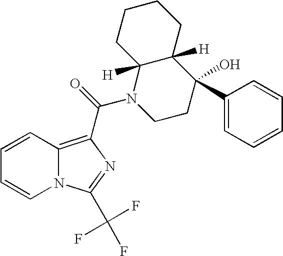 | (CIS)-4-PHENYL-1-{[3- (TRIFLUOROMETHYL)IMIDAZO[1,5- A]PYRIDIN-1- YL]CARBONYL} DECAHYDROQUINOLIN-4-OL | 444.1886 |
| 301 | 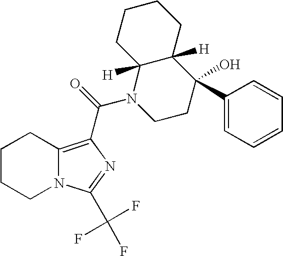 | (CIS)-4-PHENYL-1-{[3- (TRIFLUOROMETHYL)-5,6,7,8- TETRAHYDROIMIDAZO[1,5- A]PYRIDIN-1- YL]CARBONYL} DECAHYDROQUINOLIN-4-OL | 448.2181 |
| 302 | 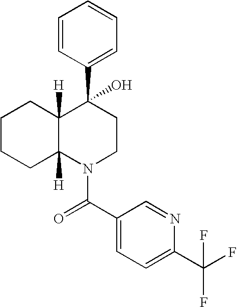 | (CIS)-4-PHENYL-1-{[6- (TRIFLUOROMETHYL)PYRIDIN-3- YL]CARBONYL} DECAHYDROQUINOLIN-4-OL | 446.2031 |
| 303 | 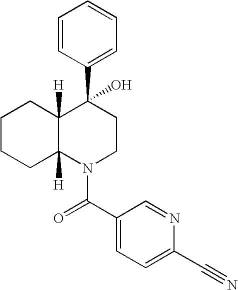 | 5-{[(CIS)-4-HYDROXY-4- PHENYLOCTAHYDROQUINOLIN- 1(2H)-YL]CARBONYL}PYRIDINE-2- CARBONITRILE | 362.1876 |
| 304 | 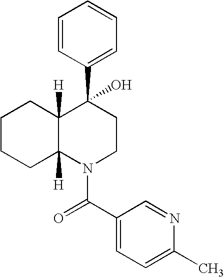 | (cis)-1-[(6-methylpyridin-3- yl)carbonyl]-4- phenyldecahydroquinolin-4-ol | 351.2064 |
| 305 | 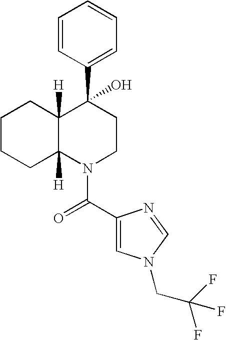 | (cis)-4-phenyl-1-{[1-(2,2,2- trifluoroethyl)-1H-imidazol-4- yl]carbonyl}decahydroquinolin-4-ol | 408.1892 |
| 306 | 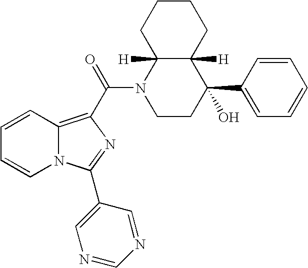 | (cis)-4-phenyl-1-[(3-pyrimidin-5- ylimidazo[1,5-a]pyridin-1- yl)carbonyl]decahydroquinolin-4-ol | 454.3 |
| 307 | 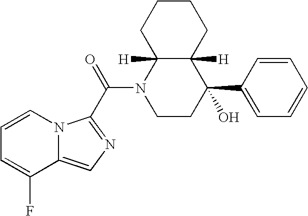 | (cis)-1-[(8-fluoroimidazo[1,5-a]pyridin- 3-yl)carbonyl]-4- phenyldecahydroquinolin-4-ol | 394.3 |
| 308 | 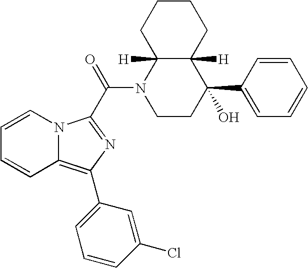 | (cis)-1-{[1-(3- chlorophenyl)imidazo[1,5-a]pyridin-3- yl]carbonyl}-4- phenyldecahydroquinolin-4-ol | 486.3 |
| 309 | 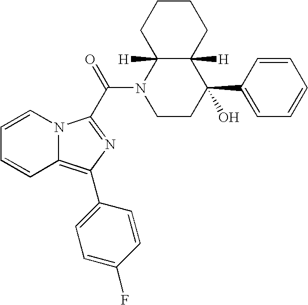 | (cis)-1-{[1-(4- fluorophenyl)imidazo[1,5-a]pyridin-3- yl]carbonyl}-4- phenyldecahydroquinolin-4-ol | 470.3 |
| 310 | 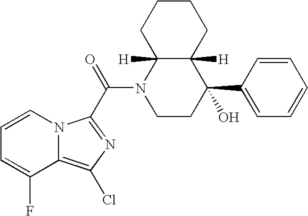 | (cis)-1-[(1-chloro-8-fluoroimidazo[1,5- a]pyridin-3-yl)carbonyl]-4- phenyldecahydroquinolin-4-ol | 410.2 |
| 311 | 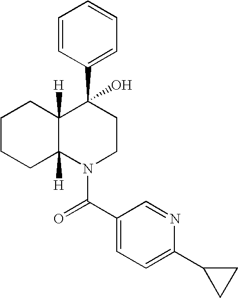 | (CIS)-1-[(6-CYCLOPROPYLPYRIDIN- 3-YL)CARBONYL]-4- PHENYLDECAHYDROQUINOLIN-4- OL | 377.2218 |
| 312 | 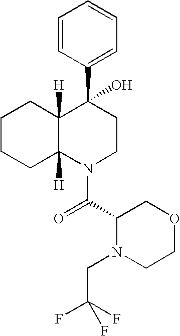 | (CIS)-4-PHENYL-1-{[(3S)-4-(2,2,2- TRIFLUOROETHYL)MORPHOLIN-3- YL]CARBONYL} DECAHYDROQUINOLIN-4-OL | 427.2201 |
| 313 | 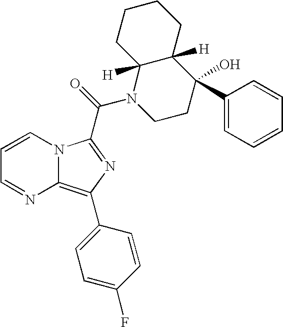 | (cis)-1-{[8-(4- fluorophenyl)imidazo[1,5-a]pyrimidin- 6-yl]carbonyl}-4- phenyldecahydroquinolin-4-ol | 471.2191 |
| 314 | 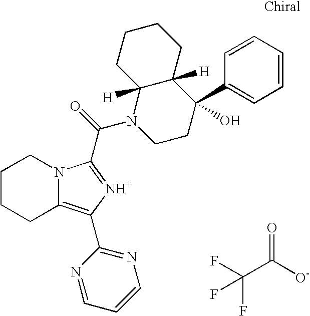 | 3-{[(cis)-4-hydroxy-4- phenyloctahydroquinolin-1(2H)- yl]carbonyl}-1-pyrimidin-2-yl-5,6,7,8- tetrahydroimidazo[1,5-a]pyridin-2-ium trifluoroacetate | 458.4 |
| 315 | 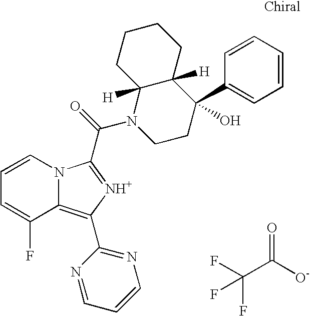 | 8-fluoro-3-{[(cis)-4-hydroxy-4- phenyloctahydroquinolin-1(2H)- yl]carbonyl}-1-pyrimidin-2- ylimidazo[1,5-a]pyridin-2-ium trifluoroacetate | 472.4 |
| 316 | 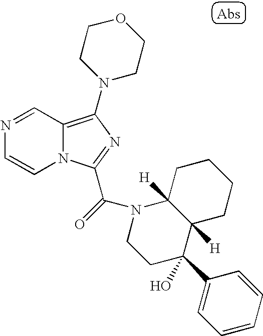 | (cis)-1-[(1-MORPHOLIN-4- YLIMIDAZO[1,5-A]PYRAZIN-3- YL)CARBONYL]-4- PHENYLDECAHYDROQUINOLIN-4- OL | 462.2481 |
| 317 | 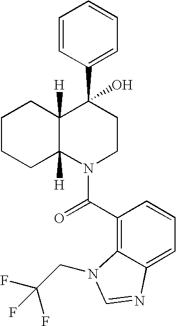 | (CIS)-4-PHENYL-1-{[1-(2,2,2- TRIFLUOROETHYL)-1H- BENZIMIDAZOL-7- YL]CARBONYL} DECAHYDROQUINOLIN-4-OL | 458.2042 |
| 318 | 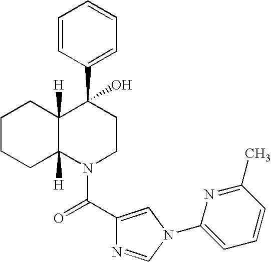 | (CIS)-1-{[1-(6-METHYLPYRIDIN-2- YL)-1H-IMIDAZOL-4- YL]CARBONYL}-4- PHENYLDECAHYDROQUINOLIN-4- OL | 417.2279 |
| 319 | 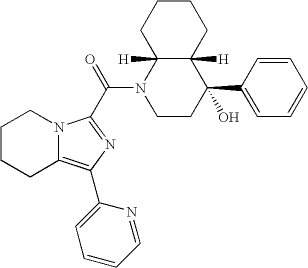 | (cis)-4-phenyl-1-[(1-pyridin-2-yl- 5,6,7,8-tetrahydroimidazo[1,5- a]pyridin-3- yl)carbonyl]decahydroquinolin-4-ol | 457.4 |
| 320 | 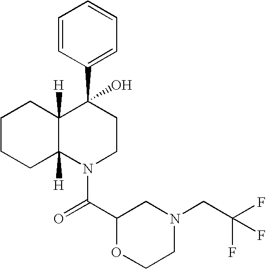 | (CIS)-4-PHENYL-1-{[4-(2,2,2- TRIFLUOROETHYL)MORPHOLIN-2- YL]CARBONYL} DECAHYDROQUINOLIN-4-OL | 427.2196 |
| 321 | 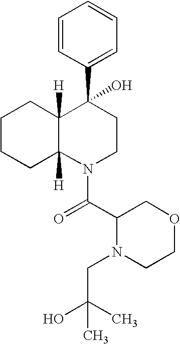 | (CIS)-1-{[(3S)-4-(2-HYDROXY-2- METHYLPROPYL)MORPHOLIN-3- YL]CARBONYL}-4- PHENYLDECAHYDROQUINOLIN-4- OL | 417.2737 |
| 322 | 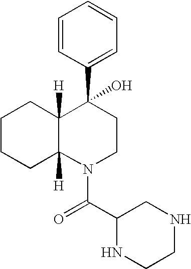 | (CIS)-4-PHENYL-1-(PIPERAZIN-2- YLCARBONYL) DECAHYDROQUINOLIN-4-OL | 344.2328 |
| 323 | 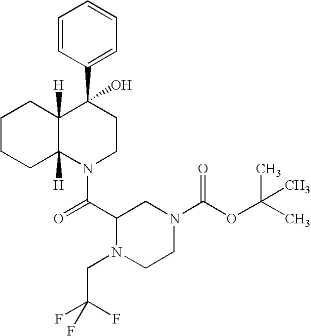 | TERT-BUTYL 3-{[(CIS)-4-HYDROXY- 4-PHENYLOCTAHYDROQUINOLIN- 1(2H)-YL]CARBONYL}-4-(2,2,2- TRIFLUOROETHYL)PIPERAZINE-1- CARBOXYLATE | 526.2867 |
| 324 | 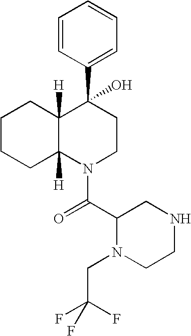 | (CIS)-4-PHENYL-1-{(1-(2,2,2- TRIFLUOROETHYL)PIPERAZIN-2- YL]CARBONYL} DECAHYDROQUINOLIN-4-OL | 426.235 |
| 325 | 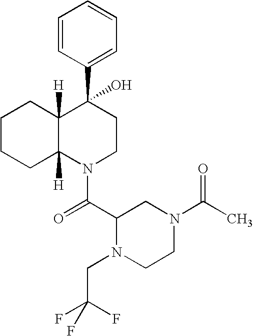 | (CIS)-1-{[4-ACETYL-1-(2,2,2- TRIFLUOROETHYL)PIPERAZIN-2- YL]CARBONYL}-4- PHENYLDECAHYDROQUINOLIN-4- OL | 468.2461 |
| 326 | 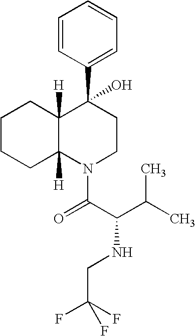 | (CIS)-4-PHENYL-1-[N-(2,2,2- TRIFLUOROETHYL)-L- VALYL]DECAHYDROQUINOLIN-4- OL | 413.2401 |
| 327 | 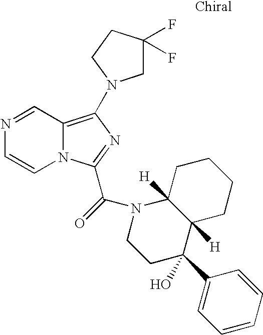 | (cis)-1-{[1-(3,3-difluoropyrrolidin-1- yl)imidazo[1,5-a]pyrazin-3- yl]carbonyl}-4- phenyldecahydroquinolin-4-ol | 482.2352 |
| 328 | 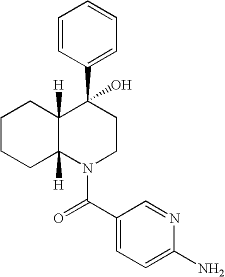 | (CIS)-1-[(6-AMINOPYRIDIN-3- YL)CARBONYL]-4- PHENYLDECAHYDROQUINOLIN-4- OL | 352.2016 |
| 329 | 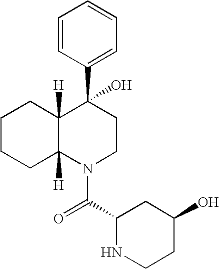 | (CIS)-1-{[(2S,4S)-4- HYDROXYPIPERIDIN-2- YL]CARBONYL}-4- PHENYLDECAHYDROQUINOLIN-4- OL | 359.2325 |
| 330 | 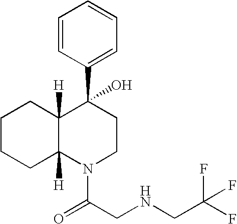 | (CIS)-4-PHENYL-1-[N-(2,2,2- TRIFLUOROETHYL)GLYCYL] DECAHYDROQUINOLIN-4-OL | 371.1933 |
| 331 | 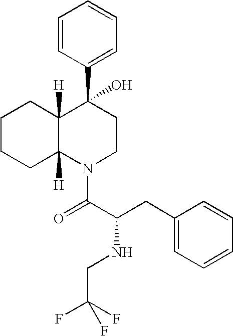 | (CIS)-4-PHENYL-1-[N-(2,2,2- TRIFLUOROETHYL)-L- PHENYLALANYL] DECAHYDROQUINOLIN-4-OL | 461.2399 |
| 332 | 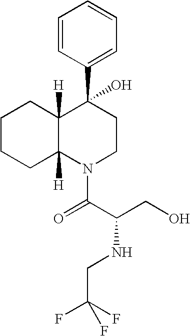 | (CIS)-1-{(2S)-3-HYDROXY-2-[(2,2,2- TRIFLUOROETHYL)AMINO] PROPANOYL}-4- PHENYLDECAHYDROQUINOLIN-4- OL | 401.2041 |
| 333 | 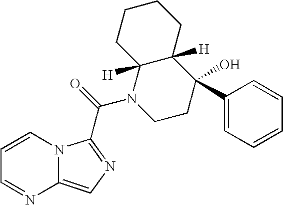 | (cis)-1-(imidazo[1,5-a] pyrimidin-6- ylcarbonyl)-4- phenyldecahydroquinolin-4-ol | 377.21 |
| 334 | 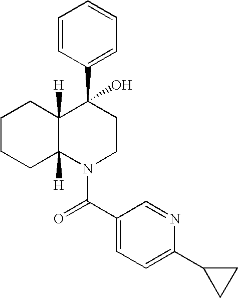 | (CIS)-1-[(6-CYCLOPROPYLPYRIDIN- 3-YL)CARBONYL]-4- PHENYLDECAHYDROQUINOLIN-4- OL | 377.2219 |
| 335 | 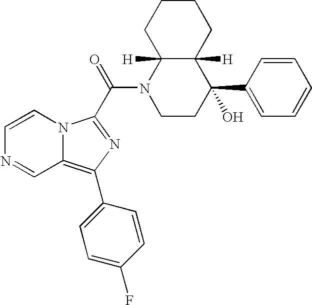 | (cis)-1-{[1-(4- fluorophenyl)imidazo[1,5-a]pyrazin-3- yl]carbonyl}-4- phenyldecahydroquinolin-4-ol | 471.16 |
| 336 | 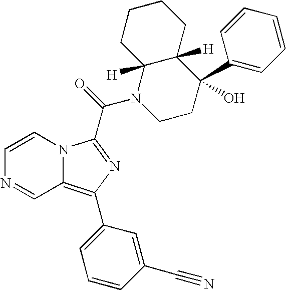 | 3-(3-{[(cis)-4-hydroxy-4- phenyloctahydroquinolin-1(2H)- yl]carbonyl}imidazo[1,5-a]pyrazin-1- yl)benzonitrile | 478.17 |
| 337 | 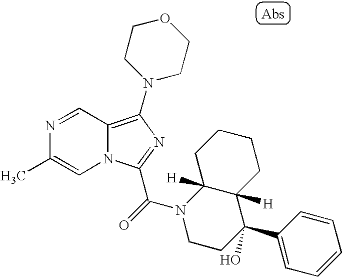 | (cis)-1-[(6-METHYL-1-MORPHOLIN- 4-YLIMIDAZO[1,5-A]PYRAZIN-3- YL)CARBONYL]-4- PHENYLDECAHYDROQUINOLIN-4- OL | 476.2637 |
| 338 | 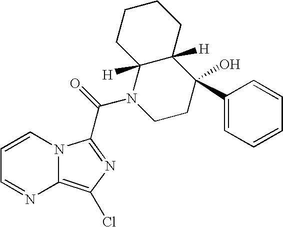 | (cis)-1-[(8-chloroimidazo[1,5- a]pyrimidin-6-yl)carbonyl]-4- phenyldecahydroquinolin-4-ol | 411.1571 |
| 339 | 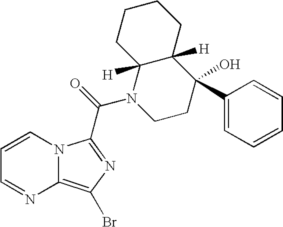 | (cis)-1-[(8-bromoimidazo[1,5- a]pyrimidin-6-yl)carbonyl]-4- phenyldecahydroquinolin-4-ol | 455.1062 |
| 340 | 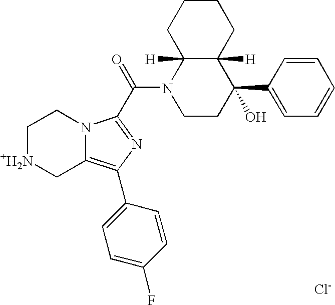 | 1-(4-fluorophenyl)-3-{[(cis)-4-hydroxy- 4-phenyloctahydroquinolin-1(2H)- yl]carbonyl}-5,6,7,8- tetrahydroimidazo[1,5-a]pyrazin-7-ium chloride | 475.4 |
| 341 | 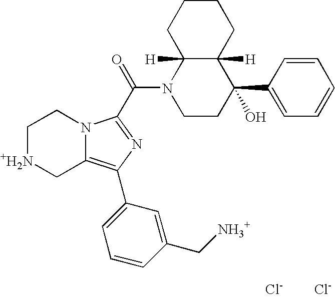 | 1-[3-(ammoniomethyl)phenyl]-3- {[(cis)-4-hydroxy-4- phenyloctahydroquinolin-1(2H)- yl]carbonyl}-5,6,7,8- tetrahydroimidazo[1,5-a]pyrazin-7-ium dichloride | 486.5 |
| 342 | 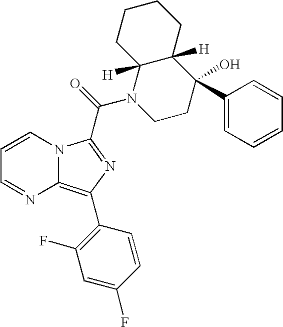 | (cis)-1-{[8-(2,4- difluorophenyl)imidazo[1,5- a]pyrimidin-6-yl]carbonyl}-4- phenyldecahydroquinolin-4-ol | 489.2075 |
| 343 | 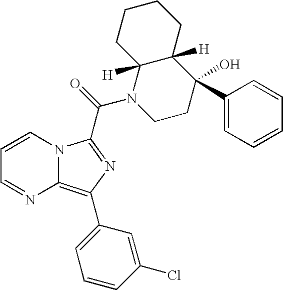 | (cis)-1-{[8-(3- chlorophenyl)imidazo[1,5-a]pyrimidin- 6-yl]carbonyl}-4- phenyldecahydroquinolin-4-ol | 487.1876 |
| 344 | 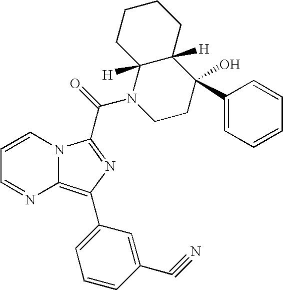 | 3-(6-{[(cis)-4-hydroxy-4- phenyloctahydroquinolin-1(2H)- yl]carbonyl}imidazo[1,5-a]pyrimidin-8- yl)benzonitrile | 478.2218 |
| 345 | 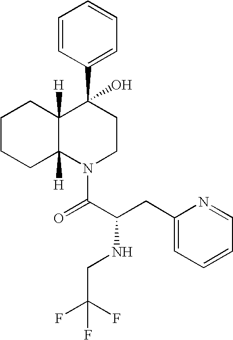 | (CIS)-4-PHENYL-1-[3-PYRIDIN-2-YL- N-(2,2,2-TRIFLUOROETHYL)-L- ALANYL]DECAHYDROQUINOLIN-4- OL | 462.2394 |
| 346 | 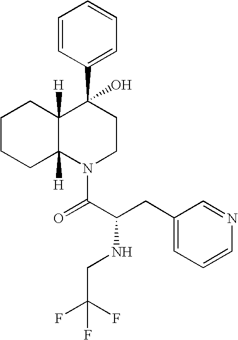 | (CIS)-4-PHENYL-1-[3-PYRIDIN-3-YL- N-(2,2,2-TRIFLUOROETHYL)-L- ALANYL]DECAHYDROQUINOLIN-4- OL | 462.2391 |
| 347 | 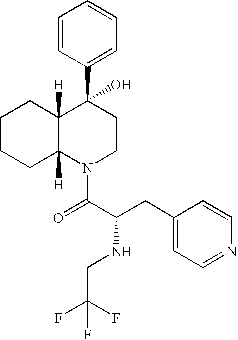 | (CIS)-4-PHENYL-1-[3-PYRIDIN-4-YL- N-(2,2,2-TRIFLUOROETHYL)-L- ALANYL]DECAHYDROQUINOLIN-4- OL | 462.2392 |

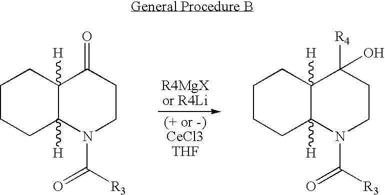

### Example II-1 (General Procedure B1)

### (Trans)-1-benzoyl-4-pyridin-2-yldecahydroquinolin-4-ol

To a solution of 2-bromopyridine (0.057 ml, 0.583 mmol) in dry THF (1 ml) was added nBuLi (0.233 ml, 0.583 mmol) slowly at −78° C. After 30 min (trans)-1-benzoyloctahydroquinolin-4(1H)-one (50 mg, 0.194 mmol) was added all at once as a solid. The cooling bath was removed and the mixture allowed to warm to RT. After 2 hr the mixture was diluted with saturated aqueous NH₄Cl and extracted with EtOAc (3×). The combined organic layers were dried (MgSO₄), filtered, and concentrated. Flash column chromatography (50% EtOAc/hexanes) gave the title compound as a pale yellow foam (15 mg, 23%). ¹H-NMR (CDCl₃, 500 MHz) δ 8.51 (d, J=4.88 Hz, 1H), 7.73 (dt, J=1.71 and 5.86 Hz, 1H), 7.51 (m, 2H), 7.39 (m, 4H), 7.21 (dt, J=0.74 and 4.15 Hz, 1H), 3.92 (dt, J=3.42 and 7.81 Hz, 1H), 3.82 (m, 1H), 3.60 (m, 1H), 2.22 (m, 1H), 2.15-2.05 (m, 2H), 1.93-1.84 (m, 2H), 1.73 (m, 1H), 1.59 (m, 1H), 1.33-1.14 (m, 3H), 0.87 (m, 1H). HRMS (M+H)⁺ 337.1910.

### Example II-2 (General Procedure B2)

### (Trans)-1-benzoyl-4-(3-methoxyphenyndecahydroquinolin-4-ol

Anhydrous cerium chloride beads (71.8 mg, 0.291 mmol) were ground under N₂ then suspended in dry THF (1 ml). To this was added (trans)-1-benzoyloctahydroquinolin-4(1H)-one (50 mg, 0.194 mmol) all at once as a solid at RT. After 2 hr 3-methoxyphenylmagnesium bromide (0.291 ml, 0.291 mmol) was added at RT. After 90 min. the mixture was diluted with saturated aqueous NH₄Cl and extracted with CH₂Cl₂ (3×). The combined organic layers were dried (MgSO₄), filtered, and concentrated. Flash column chromatography (40% EtOAc/hexanes) gave the title compound an off-white foam (34 mg, 48%). ¹H-NMR (CDCl₃, 500 MHz) δ 7.46 (m, 2H), 7.39 (m, 3H), 7.29 (m, 1H), 6.98 (m, 1H), 6.93 (d, J=7.81 Hz, 1H), 6.77 (dd, J=1.96 and 7.57 Hz, 1H), 3.95 (dt, J=3.42 and 11.23 Hz, 1H), 3.82 (s, 3H), 3.80 (m, 1H), 3.53 (m, 1H), 2.27 (m, 1H), 2.11 (m, 2H), 1.94 (m, 1H), 1.83 (s, 1H), 1.75 (m, 1H), 1.66 (m, 1H), 1.38-1.07 (m, 4H). HRMS (M+H)⁺ 366.2073.

Compounds in Table II were synthesized using Steps 3 and 4 of Scheme 1 and General Scheme A, General Scheme B1 or General Scheme B2.

TABLE II

| Example | Structure | Name | MS (M + H)+ |
|---|---|---|---|
| 257 | 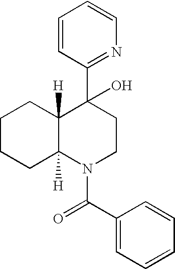 | 1-benzoyl-4-pyridin-2- yldecahydroquinolin-4- ol | 337.191 |
| 258 | 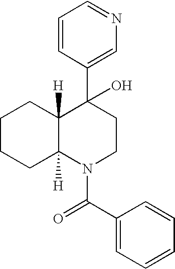 | 1-benzoyl-4-pyridin-3- yldecahydroquinolin-4- ol | 337.1911 |
| 259 | 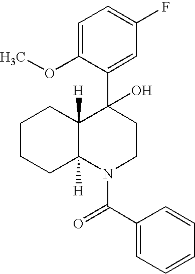 | 1-benzoyl-4-(5-fluoro-2- methoxyphenyl)decahydro- quinolin-4-ol | 384.1973 |
| 260 | 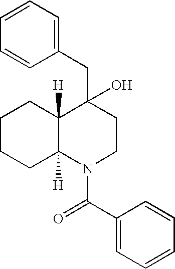 | 1-benozyl-4- benzyldecahydroquinolin- 4-ol | 350.2118 |
| 261 | 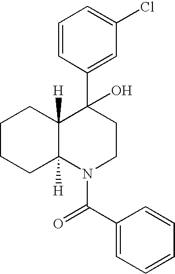 | 1-benzoyl-4-(3- chlorophenyl)decahydro quinolin-4-ol | 370.1574 |
| 262 | 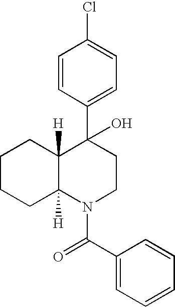 | 1-benzoyl-4-(4- chlorophenyl)decahydro quinolin-4-ol | 370.1574 |
| 263 | 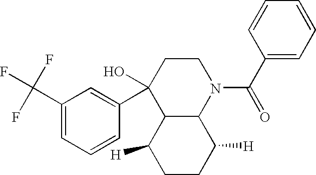 | 1-benzoyl-4-[3- (trifluoromethyl)phenyl] decahydroquinolin-4-ol | 404.1836 |
| 264 | 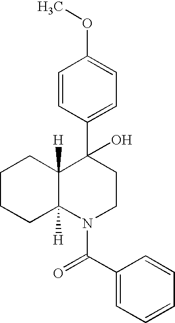 | 1-benzoyl-4-(4- methoxyphenyl)decahydro- quinolin-4-ol | 366.207 |
| 265 | 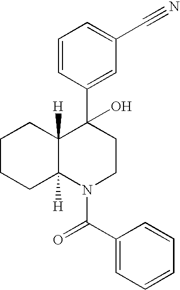 | 3-[1-benzoyl-4- hydroxydecahydroquinolin- 4-yl]benzontrile | 361.1915 |
| 266 | 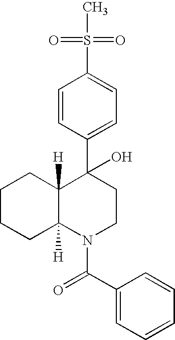 | 1-benozyl-4-[4- (methylsulfonyl)phenyl] decahydroquinolin-4-ol | 414.1743 |
| 267 | 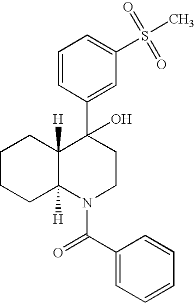 | 1-benzoyl-4-[3- (methylsulfonyl)phenyl] decahydroquinolin-4-ol | 414.1746 |
| 348 | 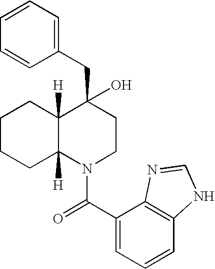 | (CIS)-1-(1H- BENZIMIDAZOL-4- YLCARBONYL)-4- BENZYLDECAHYDROQUINO- LIN-4-OL | 390.2169 |
| 349 | 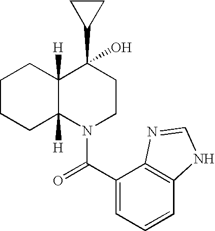 | (CIS)-1-(1H- BENZIMIDAZOL-4- YLCARBONYL)-4- CYCLOPROPYLDECAHYDRO- QUINOLIN-4-ol | 340.2019 |
| 350 | 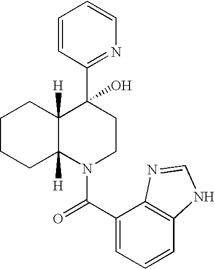 | (CIS)-1-(1H- BENZIMIDAZOL-4- YLCARBONYL)-4- PYRIDIN-2- YLDECAHYDROQUINOLIN- 4-OL | 377.1963 |
| 351 | 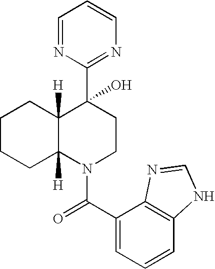 | (CIS)-1-(1H- BENZIMIDAZOL-4- YLCARBONYL)-4- PYRIMIDIN-2- YLDECAHYDROQUINOLIN- 4-OL | 378.1919 |
| 352 | 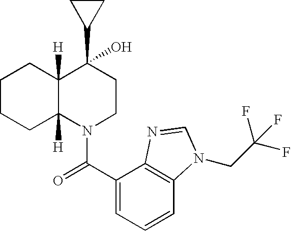 | (CIS)-4-CYCLOPROPYL- 1-{[1-(2,2,2- TRIFLUOROETHYL)-1H- BENZIMIDAZOL-4- YL]CARBONYL}DECAHYDRO- QUINOLIN-4-Ol | 422.2051 |
| 353 | 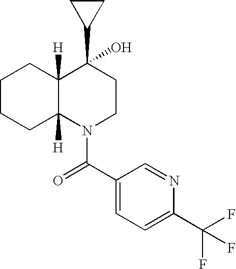 | (CIS)-4-CYCLOPROPYL- 1-{[6- (TRIFLUOROMETHYL)PY RIDIN-3- YL]CARBONYL}DECAHYDRO- QUINOLIN-4-OL | 369.1784 |
| 354 | 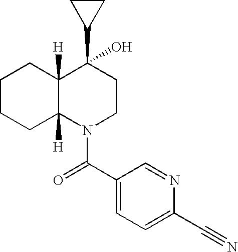 | 5-{[(CIS)-4- CYCLOPROPYL-4- HYDROXYOCTAHYDROQUINO- LIN-1(2H)- YL]CARBONYL}PYRIDINE- 2-CARBONITRILE | 326.1861 |
| 355 | 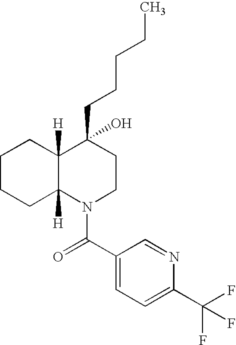 | (CIS)-4-PENTYL-1-{[6- (TRIFLUOROMETHYL)PY RIDIN-3- YL]CARBONYL}DECAHYDRO- QUINOLIN-4-OL | 399.2247 |
| 356 | 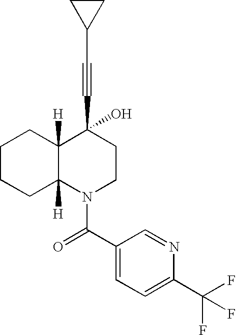 | (CIS)-4- (CYCLOPROPYLETHYNYL)- 1-{[6- (TRIFLUOROMETHYL)PY RIDIN-3- YL]CARBONYL}DECAHYDRO- QUINOLIN-4-OL | 393.1784 |
| 357 | 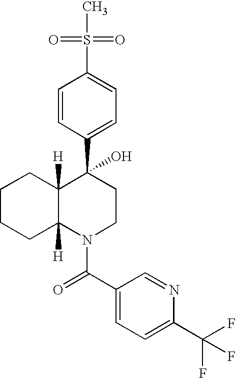 | (CIS)-4-[4- (METHYLSULFONYL)PHE NYL]-1-{[6- (TRIFLUOROMETHYL)PY RIDIN-3- YL]CARBONYL}DECAHYDRO- QUINOLIN-4-OL | 482.9 |
| 358 | 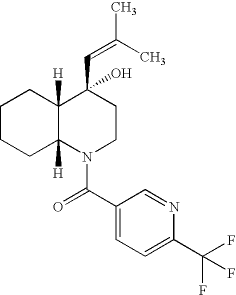 | (CIS)-4-(2-METHYLPROP- 1-EN-1-YL)-1-{[6- (TRIFLUOROMETHYL)PY RIDIN-3- YL]CARBONYL}DECAHYDRO- QUINOLIN-4-OL | 383.1963 |
| 359 | 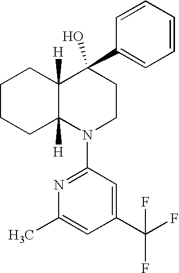 | (CIS)-1-[6-METHYL-4- (TRIFLUOROMETHYL)PY RIDIN-2-yL]-4- PHENYLDECAHYDROQUINO- LIN-4-OL | 391.1972 |

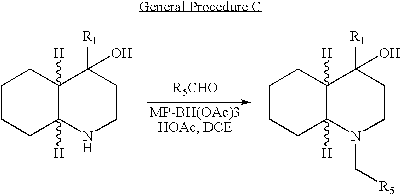

### General Procedure C: Reductive alkylation of (cis)-4-phenyldecahydroquinolin-4-ol with aldehydes

The oxalate salt of (cis)-4-phenyldecahydroquinolin-4-ol (0.16 mmole) was taken up in 1 mL DCE. To this was added DIEA (0.16 mmole), HOAc (0.47 mmole), an aldehyde (0.32 mmole) and MP-BH(OAc)₃ resin (220 mg, 0.47 mmole). After agitating for 4-8 hr the mixture was filtered, eluting with CH₂Cl₂, and concentrated. The residue was purified by reverse-phase HPLC on a Waters Sunfire column (20×150 mm, 5 μm) with gradient elution using 5-50% CH₃CN/water containing 0.1% TFA over 20 minutes. Fractions containing the product were pooled and concentrated. The residue was taken up in CH₂Cl₂ and treated with PS-DIEA resin. The mixture was filtered and concentrated to give the desired compound.

Compounds in Table III were synthesized using General Procedure C.

TABLE III

| Example | Structure | Name | MS (M + H)+ |
|---|---|---|---|
| 268 | 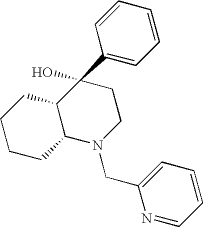 | 4-phenyl-1-(pyridin-2- ylmethyl)decahydroquinolin- 4-ol | 323.2117 |
| 269 | 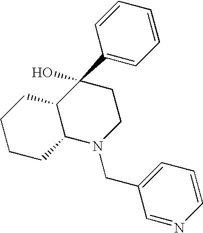 | 4-phenyl-1-(pyridin-3- ylmethyl)decahydroquinolin- 4-ol | 323.2117 |
| 270 | 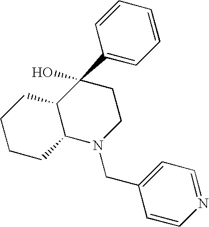 | 4-phenyl-1-(pyridin-4- ylmethyl)decahydroquinolin- 4-ol | 323.2117 |
| 271 | 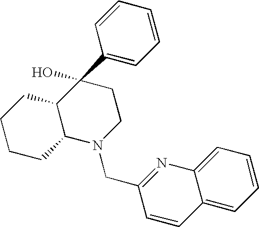 | 4-phenyl-1-(quinolin-4- ylmethyl)decahydroquinolin- 4-ol | 373.227 |
| 272 | 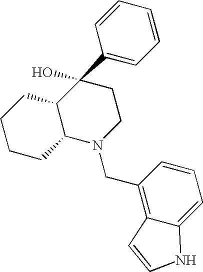 | 1-(1H-indol-4-ylmethyl)- 4- phenyldecahydroquinolin- 4-ol | 361.2274 |
| 273 | 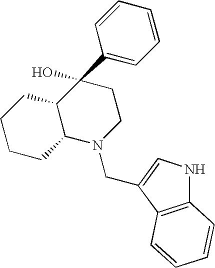 | 1-(1H-indol-3-ylmethyl)- 4- phenyldecahydroquinolin- 4-ol | 361.2272 |
| 274 | 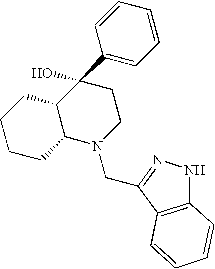 | 1-(1H-indazol-3- ylmethyl)-4- phenyldecahydroquinolin- 4-ol | 362.2226 |
| 275 | 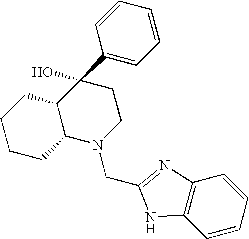 | 1-(1H-benzimidazol-2- ylmethyl)-4- phenyldecahydroquinolin- 4-ol | 362.2226 |
| 276 | 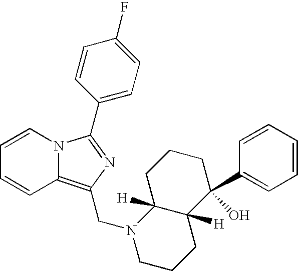 | (cis)-1-{[3-(4- fluorophenyl)imidazo[1,5- a]pyridin-1-yl]methyl}-5- phenyldecahydroquinolin- 5-ol | 456.1 |

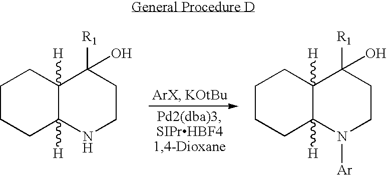

### Example IV-1 (General Procedure D)

### (Cis)-4-phenyl-1-[4-(trifluoromethyl)pyridin-2-yl]decahydroquinolin-4-ol

To (cis)-4-phenyldecahydroquinolin-4-ol (25 mg, 0.108 mmol) was added 2-chloro-4-(trifluoromethyl)pyridine (21.6 mg, 0.119 mmol), potassium tertiary-butoxide (18.2 mg, 0.162 mmol), tris(dibenzylideneacetone)dipalladium(0) (9.9 mg, 0.011 mmol), and 1,3-bis(2,6-di-1-propylphenyl)-4,5-dihydroimidazolium tetrafluoroborate (10.3 mg, 0.022 mmol). The mixture was taken up in 1 mL 1,4-dioxane, degassed by bubbling nitrogen through solution, and then heated to 100° C. for 17 hours. The reaction mixture was filtered through a 0.45 μm syringe-tip filter, washing with CH₂Cl₂. The filtrate was evaporated under nitrogen and purified by reversed phase HPLC (5% to 75% CH₃CN (0.1% TFA) in water (0.1% TFA) over 20 minutes at 20 mL/minute, 21.2 mm×100 mm Phenomenex Gemini C18). The desired fractions were free-based using a Phenomenex Strata X-C cartridge and concentrated in vacuo to give the title compound (10.2 mg, 20%). ¹H NMR (CDCl₃, 400 MHz) δ 8.31 (1H, d, J=5.21 Hz), 7.47 (2H, d, J=7.51 Hz), 7.37 (2H, t, 7.70 Hz), 7.29-7.26 (1H, m), 6.79 (1H, s), 6.72 (1H, d, J=5.13 Hz), 4.39-4.29 (2H, br m), 3.53 (1H, dt, J=13.01, 2.20 Hz), 2.34-2.20 (3H, m), 1.90-1.88 (1H, m), 1.79-1.68 (2H, m), 1.62-1.58 (2H, m), 1.49-1.26 (3H, m). HRMS (M+H)⁺ 377.1813.

Compounds in Table IV were synthesized using General Procedure D.

TABLE IV

| | | | MS |
|---|---|---|---|
| Example | Structure | Name | (M + H)+ |
| 277 | 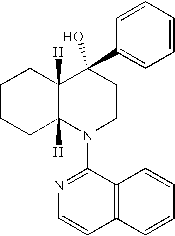 | (cis)-1-isoquinolin-1-yl-4- phenyldecahydroquinolin-4-ol | 359.2114 |
| 278 | 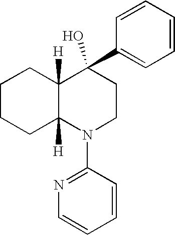 | (cis)-4-phenyl-1-pyridin-2- yldecahydroquinolin-4-ol | 309.1934 |
| 279 | 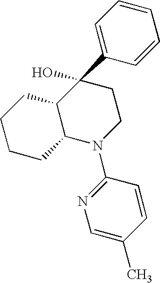 | (cis)-1-(5-methylpyridin-2-yl)-4- phenyldecahydroquinolin-4-ol | 323.2095 |
| 280 | 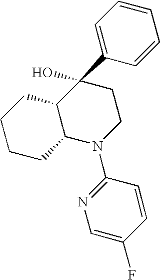 | (cis)-1-(5-fluoropyridin-2-yl)-4- phenyldecahydroquinolin-4-ol | 327.1843 |
| 281 | 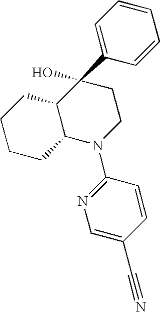 | 6-[(cis)-4-hydroxy-4- phenyloctahydroquinolin-1(2H)- yl]nicotinonitrile | 334.189 |
| 282 | 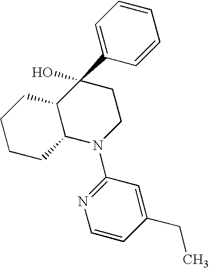 | (CIS)-1-(4-ethylpyridin-2-yl)-4- phenyldecahydroquinolin-4-ol | 337.225 |
| 283 | 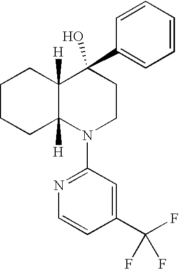 | (cis)-4-phenyl-1-[4- (trifluoromethyl)pyridin-2- yl]decahydroquinolin-4-ol | 377.1813 |
| 284 | 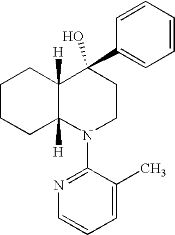 | (cis)-1-(3-methylpyridin-2-yl)-4- phenyldecahydroquinolin-4-ol | 323.2093 |
| 285 | 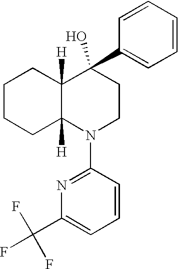 | (cis)-4-phenyl-1-[5- (trifluoromethyl)pyridin-2- yl]decahydroquinolin-4-ol | 377.1817 |
| 286 | 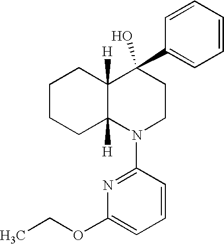 | (cis)-1-(6-ethoxypyridin-2-yl)-4- phenyldecahydroquinolin-4-ol | 353.2202 |
| 287 | 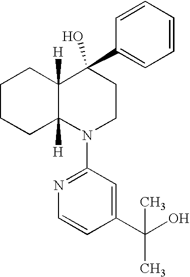 | (cis)-1-[4-(1-hydroxy-1- methylethyl)pyridin-2-yl]-4- phenyldecahydroquinolin-4-ol | 367.2359 |
| 288 | 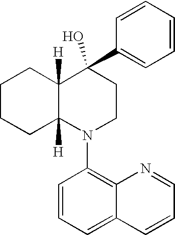 | (cis)-4-phenyl-3,4,4a,5,6,7,8,8a- octahydro-2h-1,8′-biquinolin-4-ol | 359.2098 |
| 360 | 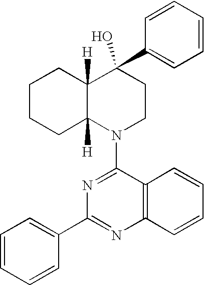 | (CIS)-4-PHENYL-1-(2- PHENYLQUINAZOLIN-4- YL)DECAHYDROQUINOLIN-4-OL | 436.2367 |
| 361 | 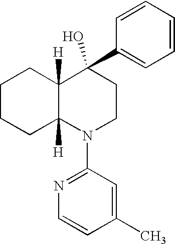 | (CIS)-1-(4-METHYLPYRIDIN-2-YL)-4- PHENYLDECAHYDROQUINOLIN-4-OL | 323.2093 |
| 362 | 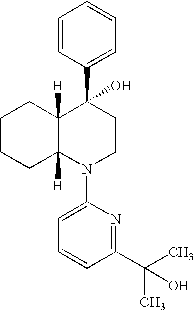 | (CIS)-1-[6-(1-HYDROXY-1- METHYLETHYL)PYRIDIN-2-YL]-4- PHENYLDECAHYDROQUINOLIN-4-OL | 367.2359 |
| 363 | 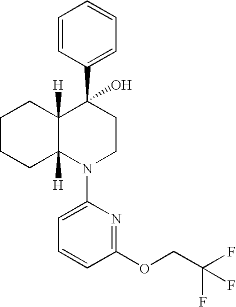 | (CIS)-4-PHENYL-1-[6-(2,2,2- TRIFLUOROETHOXY)PYRIDIN-2- YL]DECAHYDROQUINOLIN-4-OL | 407.1916 |
| 359 | 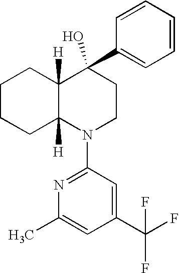 | CIS)-1-[6-METHYL-4- (TRIFLUOROMETHYL)PYRIDIN-2-YL]-4- PHENYLDECAHYDROQUINOLIN-4-OL | 391.1972 |

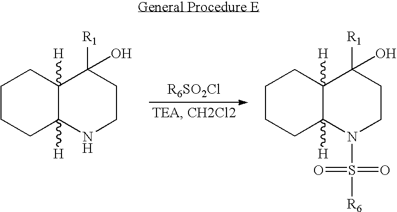

### Example V-1 (General Procedure E)

### (Cis)-1-[(1-methyl-1H-imidazol-4-yl)sulfonyl]-4-phenyldecahydroquinolin-4-ol

To a suspension of the oxalate salt of (cis)-4-phenyldecahydroquinolin-4-ol (50 mg, 0.16 mmole) in CH₂Cl₂ (0.75 mL) was added TEA (87 mL, 0.62 mmole) then 1-methyl-1H-imidazole-4-sulfonyl chloride (37 mg, 0.20 mmole). After 48 hr the mixture was concentrated. The residue was purified by flash column chromatography to give the title compound (36 mg, 62%). ¹H-NMR (CDCl₃, 500 MHz) δ 7.52 (s, 1H), 7.45 (s, 1H), 7.38 (m, 4H), 7.25 (m, 1H), 4.09 (m, 1H), 3.90 (m, 1H), 3.76 (s, 3H), 3.65 (dt, J=2.20 and 13.19 Hz, 1H), 2.32 (m, 2H), 2.15 (m, 1H), 1.80 (m, 1H), 1.66 (m, 2H), 1.47-1.17 (m, 5H). HRMS (M+H)⁺ 376.1710.

Compounds in Table V were synthesized using General Procedure E.

TABLE V

| Ex- | | | MS |
|---|---|---|---|
| ample | Structure | Name | (M + H)+ |
| 289 | 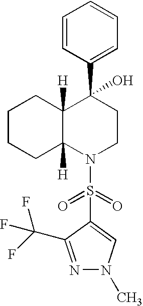 | 1-{[1-methyl-3- (trifluoromethyl)-1h- pyrazol-4-yl]sulfonyl}-4- phenyldecahydroquinolin- 4-ol | 444.1565 |
| 290 | 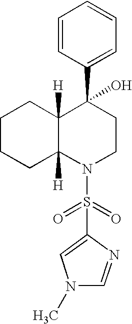 | 1-[(1-methyl-1h-imidazol- 4-yl)sulfonyl]-4- phenyldecahydroquinolin- 4-ol | 376.171 |
| 291 | 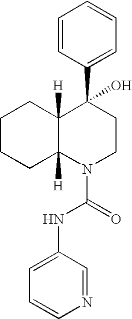 | 4-hydroxy-4-phenyl-n- pyridin-3- yloctahydroquinoline- 1(2H)-carboxamide | 352.203 |

What is claimed is:

1. A compound of Formula (I): 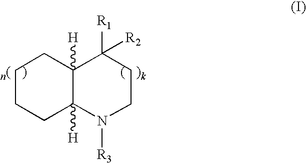
or pharmaceutically acceptable salts and N-oxides thereof, wherein.
m is 1, 2, 3 or 4;
n and k are each independently selected from 0, 1 and 2;
R¹ is independently selected from
(1) H,
(2) OH,
(3) —NH₂,
(4) —NHR⁴,
(5) —NR⁴R⁵,
(6) —CF₃,
(7) —CN,
(8) —C₁₋₆alkyl,
(9) —C₂₋₆alkenyl,
(10) —C₂₋₆alkynyl,
(11) —C₃₋₆cycloalkyl,
(12) heterocycle,
(13) heteroaryl,
(14) aryl,
wherein the alkyl, cycloalkyl, alkenyl and alkynyl of choices (8), (9), (10) and (11) are each independently optionally mono- or di-substituted with substituents independently selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, and
the heterocycle, heteroaryl and aryl of choices (12), (13), and (14), are each optionally mono- or di-substituted with substituents selected from halo, —CN, hydroxy, oxo, —C₁₋₄alkyl, —C₃₋₆cycloalkyl, —CF₃, —CHF₂, —CH₂F, —OC₁₋₆alkyl, —O—CF₃, —O—C₁₋₃alkyl-CF₃, -hydroxyC₁₋₆alkyl, —S(O)₂—R⁶, —C(O)—O—C₁₋₆alkyl, —C(O)—NR⁶R⁷, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —C₁₋₂alkyl-heterocycle, —NR⁶R⁷, —NH—C(O)—R⁶, —NH—(O)—NR⁶R⁷, —NH—S(O)₂—R⁶, —NH—C₁₋₄alkyl-aryl, and —S—C₁₋₄alkyl, wherein the aryl, heteroaryl and heterocycle portion of the substituents on choices (12), (13) and (14) are each optionally mono or di-substituted with substituents independently selected from halo, —CH₃, —CF₃, —CN, hydroxy and —OC₁₋₄alkyl;
R² is selected from the group consisting of:
(1) —C₁₋₆alkyl,
(2) —C₂₋₆alkenyl,
(3) —C₂₋₆alkynyl,
(4) —C₃₋₆cycloalkyl,
(5) carbocycle,
(6) -aryl,
(7) (CH₂)m-carbocycle,
(8) —(CH₂)m-aryl,
(9) —(CH₂)m-heterocycle,
(10) —(CH₂)m-heteroaryl,
(11) —NHR⁴,
(12) —O—C₁₋₆alkyl,
(13) —N(R⁴)C₁₋₆alkyl,
(14) —O—C₂₋₆alkenyl,
(15) —N(R⁴)C₂₋₆alkenyl,
(16) —O—C₂₋₆alkenyl,
(17) —N(R⁴)C₂₋₆alkynyl,
(18) —O—C₃₋₆cycloalkyl,
(19) —NR⁴)—C₃₋₆cycloalkyl,
(20) —O-carbocycle,
(21) N(R⁴)-carbocycle,
(22) -heterocycle,
(23) —O-heterocycle,
(24) —N(R⁴)-heterocycle,
(25) -heteroaryl,
(26) —O-heteroaryl,
(27) —N(R⁴)-heteroaryl,
(28) —O-aryl,
(29) —N(aryl)R⁴,
wherein the alkyl, cycloalkyl, carbocycle, alkenyl and alkynyl of choices (1), (2), (3), (4), (5), (7), (12), (13), (14), (15), (16), (17), (18), (19), (20), and (21) are each optionally mono- or di-substituted with substituents independently selected from hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —NR⁴R⁵, —CO₂H, CO₂R⁴, —C(O)NR⁴R⁵, aryl, heteroaryl, C₃₋₆cycloalkyl and
wherein the aryl, heteroaryl and heterocycle of choices (6), (8), (9), (10), (22), (23), (24), (25), (26), (27), (28), and (29) are optionally mono-, di- or tri-substituted with substituents independently selected from —CN, hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —C₁₋₄alkyl-OH, —S(O)₂—R⁶, —NR⁴R⁵, —CO₂H, CO₂R⁴, —C(O)NR⁴R⁵, aryl, heteroaryl, C₃₋₆cycloalkyl; or
R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle;
R³ is selected from the group consisting of:
(1) —C₁₋₆alkyl,
(2) —C(O)H,
(3) —S(O)₂H,
(4) —C(O)NHR⁵,
(5) —C(O)—C₁₋₆alkyl,
(6) —S(O)₂—C₁₋₆alkyl,
(7) —C(O)N(R⁵)—C₁₋₆alkyl,
(8) —CH₂—C₂₋₆alkenyl,
(9) —C(O)—C₂₋₆alkenyl,
(10) —S(O)₂—C₂₋₆alkenyl,
(11) —C(O)N(R⁵)—C₂₋₆alkenyl,
(12) —CH₂—C₂₋₆alkynyl,
(13) —C(O)—C₂₋₆alkynyl,
(14) —S(O)₂—C₂₋₆alkynyl,
(15) —C(O)N(R⁵)—C₂₋₆alkynyl,
(16) C₃₋₆cycloalkyl,
(17) —CH₂—C₃₋₆cycloalkyl,
(18) —C(O)—C₃₋₆cycloalkyl,
(19) —S(O)₂—C₃₋₆cycloalkyl,
(20) —C(O)N(R⁵)—C₃₋₆cycloalkyl,
(21) heterocycle,
(22) —CH₂-heterocycle,
(23) —C(O)-heterocycle,
(24) —C(O)—C₁₋₂alkyl-heterocycle,
(25) —S(O)₂-heterocycle,
(26) —C(O)N(R⁵)-heterocycle,
(27) heteroaryl,
(28) —CH₂-heteroaryl,
(29) —C(O)-heteroaryl,
(30) —C(O)—C₁₋₂alkyl-heteroaryl,
(31) —S(O)₂-heteroaryl,
(32) —C(O)N(R⁵)-heteroaryl,
(33) aryl,
(34) —CH₂-aryl,
(35) —C(O)-aryl,
(36) —C(O)—C₁₋₂alkyl-aryl,
(37) —S(O)₂-aryl,
(38) —C(O)N(R⁵)-aryl,
(39) carbocycle,
(40) —CH₂-carbocycle,
(41) —C(O)-carbocycle,
(42) —S(O)₂-carbocycle,
(43) —C(O)N(R⁵)-carbocycle,
wherein the alkyl, cycloalkyl, carbocycle, alkenyl and alkynyl of choices (1), (5), (6), (7), (8), (9), (10), (11), (12), (13), (14), (15), (16), (17) (18) (19), (20), (39), (40), (41), (42) and (43) are each optionally mono- or di-substituted with substituents independently selected from halo, oxo, hydroxy, —CN, —C₁₋₆alkyl, —CF₃, —CHF₂, CH₂F, —C₁₋₄alkyl-CF₃, —C₁₋₄alkylCHF₂, —C₁₋₄alkylCH₂F, —OC₁₋₆alkyl, —O—CF₃, —C₁₋₄alkylCF₃, hydroxyC₁₋₆alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NH₂, —NH—C(O)—R⁶, —NH—C(O)—C₃₋₆cycloalkyl, —NH—C(O)—C₁₋₄alkyl, —NH—C(O)—NR⁶R⁷, —NH—S(O)₂—R⁶, —S-heteroaryl, —S-heterocycle, aryl, —C(O)-aryl, heteroaryl, —C(O)-heteroaryl, heterocycle, and —C(O)heterocycle, wherein the aryl, heteroaryl, heteroaryl and heterocycle portion of substituents on choices (5) to (19) and (35) to (38) are each optionally mono or di-substituted with substituents independently selected from halo, —CH₃, —CF₃, —CN, hydroxy and —OC₁₋₆alkyl, and
wherein the aryl, heteroaryl and heterocycle of choices (21), (22), (23), (24), (25), (26), (27), (28), (29), (30), (31), (32), (33), (34), (35), (36), (37) and (38) is optionally mono-, di- or tri-substituted with substituents independently selected from —CN, oxo, —C₁₋₄alkyl, —C₃₋₆cycloalkyl, —CF₃, —CHF₂, —OC₁₋₆alkyl, —O—CF₃, —O—CH₂—CF₃, hydroxy, -hydroxyC₁₋₆alkyl, halo, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —CH₂-heterocycle, —NR⁶R⁷, —NH—C(O)—R⁶, —NH—C(O)—N(CH₃)₂, —NH—S(O)₂—R⁶, —NH—C₁₋₂alkyl-aryl, and —S—C₁₋₃alkyl, wherein the aryl, heteroaryl and heterocycle portion of the substituents on choices (21), (22), (23), (24), (25), (26), (27), (28), (29), (30), (31), (32), (33), (34), (35), (36), (37) and (38) are each optionally mono or di-substituted with substituents independently selected from halo, —CH₃, —CF₃, —CN, hydroxy and —OC₁₋₄alkyl;
R⁴ and R⁵ are each independently selected from
(1) H,
(2) —CF₃,
(3) —CN,
(4) —C1-6alkyl,
(5) —C2-6alkenyl,
(6) —C2-6alkynyl,
(7) —C₃₋₆cycloalkyl,
(8) heterocycle,
(9) heteroaryl,
(10) aryl,
wherein the alkyl, cycloalkyl, alkenyl and alkynyl of choices (4), (5), (6) and (7) (8) are each independently optionally mono- or di-substituted with substituents independently selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —C(O)—O—C₁₋₆alkyl, —C(O)—NHC₁₋₆alkyl, —C(O)—NR⁶R⁷, —C(O)—O—C(CH₃)₃, —C₃₋₆cycloalkyl, —NH₂, —NHR⁴, —NR⁴R⁵, —NH—C(O)—R⁶, —NH—C(O)—NR⁶R⁷, —NH—S(O)₂—R⁶, and
the heterocycle, heteroaryl and aryl of choices (8), (9) and (10), is each optionally mono- or di-substituted with substituents selected from halo, —CN, hydroxy, oxo, —C₁₋₄alkyl, —C₃₋₆cycloalkyl, —CF₃, —CHF₂, —CH₂F, —OC₁₋₆alkyl, —O—CF₃, —O—C₁₋₃alkyl-CF₃, -hydroxyC₁₋₆alkyl, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —C₁₋₂alkyl-heterocycle, —NR⁶R⁷, —NH—C(O)—NR⁶R⁷, —NH—S(O)₂—R⁶, —NH—C₁₋₄alkyl-aryl, and —S—C₁₋₄alkyl, wherein the aryl, heteroaryl and heterocycle portion of the substituents on choices (9), (10) and (11) are each optionally mono or di-substituted with substituents independently selected from halo, —CH₃, —CF₃, —CN, hydroxy and —OC₁₋₄alkyl;
R⁶ is selected from hydrogen, CF₃, C₁₋₄alkyl, C₃₋₆cycloalkyl, carbocycle, aryl, heterocycle and heteroaryl;
R⁷ is selected from hydrogen and C₁₋₄alkyl;
with the proviso that when R¹ is choice (2), (3), (4) or (5), then R² is other than choice (11), (12), (13), (14), (15), (16), (17), (18), (19), (20), (21), (22), (23), (24), (25), (26), (27), (28), or (29).

2. A compound of claim 1 wherein
R¹ is independently selected from
(1) H,
(2) OH,
(3) —NH₂,
(4) —NHR⁴,
(5) —NR⁴R⁵,
(6) —CF₃,
(7) —CN,
(8) heterocycle,
(9) heteroaryl,
(10) aryl,
wherein
the heterocycle, heteroaryl and aryl of choices (8), (9), and (10), are each optionally mono- or di-substituted with substituents selected from halo, —CN, —C₁₋₄alkyl, —C₃₋₆cycloalkyl, —CF₃, —OC₁₋₆alkyl, —O—CF₃, —O—C₁₋₃alkyl-CF₃, —C(O)—O—C₁₋₆alkyl, —C(O)—NR⁶R⁷.

3. A compound of claim 1 wherein
R¹ is independently selected from
(1) H,
(2) OH,
(3) —NH₂,
(6) —CF₃,
(7) —CN.

4. A compound of claim 1 wherein
R² is selected from the group consisting of:
(1) —C₁₋₆alkyl,
(2) —C₂₋₆alkenyl,
(3) —C₂₋₆alkynyl,
(4) —C₃₋₆cycloalkyl,
(5) carbocycle,
(6) -aryl,
(7) CH₂-carbocycle,
(8) —CH₂-aryl,
(9) —CH₂-heterocycle,
(10) —CH₂-heteroaryl,
(11) —NHR⁴,
(12) —O—C₁₋₆alkyl,
(13) —N(R⁴)C₁₋₆alkyl,
(14) —O—C₂₋₆alkenyl,
(15) —N(R⁴)C₂₋₆alkenyl,
(16) —O—C₂₋₆alkenyl,
(17) —N(R⁴)C₂₋₆alkynyl,
(18) —O—C₃₋₆cycloalkyl,
(19) —N(R⁴)—C₃₋₆cycloalkyl,
(20) —O-carbocycle,
(21) N(R⁴)-carbocycle,
(22) -heterocycle,
(23) —O-heterocycle,
(24) —N(R⁴)heterocycle,
(25) -heteroaryl,
(26) —O-heteroaryl,
(27) —N(R⁴)-heteroaryl,
(28) —O-aryl,
(29) —N(aryl)R⁴,
wherein the alkyl, cycloalkyl, carbocycle, alkenyl and alkynyl of choices (1), (2), (3), (4), (5), (7), (12), (13), (14), (15), (16), (17), (18), (19), (20), and (21) are each optionally mono- or di-substituted with substituents independently selected from hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —NR⁴R⁵, —CO₂H, CO₂R⁴, —C(O)NR⁴R⁵, aryl, heteroaryl, C₃₋₆cycloalkyl and
wherein the aryl, heteroaryl and heterocycle of choices (6), (8), (9), (10), (22), (23), (24), (25), (26), (27), (28), and (29) are optionally mono-, di- or tri-substituted with substituents independently selected from —CN, hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —NR⁴R⁵, —CO₂H, CO₂R⁴, —C(O)NR⁴R⁵, aryl, heteroaryl, C₃₋₆cycloalkyl; or
R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle.

5. A compound of claim 1 wherein
R² is selected from the group consisting of:
(1) —C₁₋₆alkyl,
(2) —C₂₋₆alkenyl,
(3) —C₂₋₆alkynyl,
(4) —C₃₋₆cycloalkyl,
(5) carbocycle,
(6) -aryl,
(7) CH₂-carbocycle,
(8) —CH₂-aryl,
(9) —CH₂-heterocycle,
(10) —CH₂-heteroaryl,
(11) heterocycle,
(12) heteroaryl,
wherein the alkyl, cycloalkyl, carbocycle, alkenyl and alkynyl of choices (1), (2), (3), (4), (5) and (7), are each optionally mono- or di-substituted with substituents independently selected from hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —NR⁴R⁵, —CO₂H, CO₂R⁴, —C(O)NR⁴R⁵, aryl, heteroaryl, C₃₋₆cycloalkyl and
wherein the aryl, heteroaryl and heterocycle of choices (6), (8), (9), (10), (11) and (12) are optionally mono-, di- or tri-substituted with substituents independently selected from —CN, hydroxy, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —NR⁴R⁵, —CO₂H, CO₂R⁴, —C(O)NR⁴R⁵, aryl, heteroaryl, C₃₋₆cycloalkyl; or
R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle.

6. A compound of claim 1 wherein
R² is selected from the group consisting of:
(1) —C₁₋₆alkyl,
(2) —C₃₋₆cycloalkyl,
(3) carbocycle,
(4) -aryl,
(5) heterocycle,
(6) heteroaryl,
wherein the alkyl, cycloalkyl, and carbocycle of choices (1), (2) and (3), are each optionally mono- or di-substituted with substituents independently selected from hydroxy, —CF₃, C₁₋₄alkylCF₃, aryl, heteroaryl, and
wherein the aryl, heteroaryl and heterocycle of choices (4), (5) and (6) are optionally mono-, di- or tri-substituted with substituents independently selected from —CN, halo, —CF₃, —CO₂H, CO₂R⁴, —C(O)NR⁴R⁵, aryl, heteroaryl, C₃₋₆cycloalkyl; or
R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle.

7. A compound of claim 1 wherein
R³ is selected from the group consisting of:
(1) —C₁₋₆alkyl,
(2) —C(O)H,
(3) —C(O)NHR⁵,
(4) —C(O)—C₁₋₆alkyl,
(5) —C(O)N(R⁵)—C₁₋₆alkyl,
(6) —CH₂—C₂₋₆alkenyl,
(7) —C(O)—C₂₋₆alkenyl,
(8) —C(O)N(R⁵)—C₂₋₆alkenyl,
(9) —CH₂—C₂₋₆alkynyl,
(10) —C(O)—C₂₋₆alkynyl,
(11) —C(O)N(R⁵)—C₂₋₆alkynyl,
(12) C₃₋₆cycloalkyl,
(13) —CH₂—C₃₋₆cycloalkyl,
(14) —C(O)—C₃₋₆cycloalkyl,
(15) —C(O)N(R⁵)—C₃₋₆cycloalkyl,
(16) heterocycle,
(17) —CH₂-heterocycle,
(18) —C(O)-heterocycle,
(19) —C(O)—C₁₋₂alkyl-heterocycle,
(20) —C(O)N(R⁵)-heterocycle,
(21) heteroaryl,
(22) —CH₂-heteroaryl,
(23) —C(O)-heteroaryl,
(24) —C(O)—C₁₋₂alkyl-heteroaryl,
(25) —C(O)N(R⁵)-heteroaryl,
(26) aryl,
(27) —CH₂-aryl,
(28) —C(O)-aryl,
(29) —C(O)—C₁₋₂alkyl-aryl,
(30) —C(O)N(R⁵)-aryl,
(31) carbocycle,
(32) —CH₂-carbocycle,
(33) —C(O)-carbocycle,
(34) —C(O)N(R⁵)-carbocycle,
wherein the alkyl, cycloalkyl, carbocycle, alkenyl and alkynyl of choices (1), (4), (5), (6), (7), (8), (9), (10), (11), (12), (13), (14), (15), (31), (32), (33), and (34) are each optionally mono- or di-substituted with substituents independently selected from halo, oxo, hydroxy, —CN, —C₁₋₆alkyl, —CF₃, —CHF₂, CH₂F, —C₁₋₄alkyl-CF₃, —C₁₋₄alkylCHF₂, —C₁₋₄alkylCH₂F,—OC₁₋₆alkyl, —O—CF₃, —C₁₋₄alkylCF₃, hydroxyC₁₋₆alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NH₂, —NH—C(O)—R⁶, —NH—C(O)—C₃₋₆cycloalkyl, —NH—C(O)—C₁₋₄alkyl, —NH—C(O)—NR⁶R⁷, —NH—S(O)₂—R⁶, —S-heteroaryl, —S-heterocycle, aryl, —C(O)-aryl, heteroaryl, —C(O)-heteroaryl, heterocycle, and —C(O)heterocycle, wherein the aryl, heteroaryl, heteroaryl and heterocycle portion of substituents on choices (1), (4), (5), (6), (7), (8), (9), (10), (11), (12), (13), (14), (15), (31), (32), (33), and (34) are each optionally mono or di-substituted with substituents independently selected from halo, —CH₃, —CF₃, —CN, hydroxy and —OC₁₋₆alkyl, and
wherein the aryl, heteroaryl and heterocycle of choices (16), (17), (18), (19), (20), (21), (22), (23), (24), (25), (26), (27), (28), (29) and (30), is optionally mono-, di- or tri-substituted with substituents independently selected from —CN, oxo, —C₁₋₄alkyl, —C₃₋₆cycloalkyl, —CF₃, —CHF₂, —OC₁₋₆alkyl, —O—CH₂—CF₃, hydroxy, -hydroxyC₁₋₆alkyl, halo, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —CH₂-heterocycle, —NR⁶R⁷, —NH—C(O)—R⁶, —NH—C(O)—N(CH₃)₂, —NH—S(O)₂—R⁶, —NH—C₁₋₂alkyl-aryl, and —S—C₁₋₃alkyl, wherein the aryl, heteroaryl and heterocycle portion of the substituents on choices (16), (17), (18), (19), (20), (21), (22), (23), (24), (25), (26), (27), (28), (29) and (30) is each optionally mono or di-substituted with substituents independently selected from halo, —CH₃, —CF₃, —CN, hydroxy and —OC₁₋₄alkyl.

8. A compound of claim 7 wherein
R³ is selected from the group consisting of:
(1) —C₁₋₆alkyl,
(2) —C(O)—C₁₋₆alkyl,
(3) —CH₂—C₂₋₆alkenyl,
(4) —C(O)—C₂₋₆alkenyl,
(5) —CH₂—C₂₋₆alkynyl,
(6) —C(O)—C₂₋₆alkynyl,
(7) C₃₋₆cycloalkyl,
(8) —C(O)—C₃₋₆cycloalkyl,
(9) —C(O)N(R⁵)—C₃₋₆cycloalkyl,
(10) heterocycle,
(11) —CH₂-heterocycle,
(12) —C(O)-heterocycle,
(13) —C(O)—C₁₋₂alkyl-heterocycle,
(14) heteroaryl,
(15) —CH₂-heteroaryl,
(16) —C(O)-heteroaryl,
(17) —C(O)—C₁₋₂alkyl-heteroaryl,
(18) aryl,
(19) —CH₂-aryl,
(20) —C(O)-aryl,
(21) —C(O)—C₁₋₂alkyl-aryl,
(22) carbocycle,
(23) —CH₂-carbocycle,
(24) —C(O)-carbocycle,
wherein the alkyl, cycloalkyl, carbocycle, alkenyl and alkynyl of choices (1), (2), (3), (4), (5), (6), (7), (8), (9), (22), (23) and (24) are each optionally mono- or di-substituted with substituents independently selected from halo, oxo, hydroxy, —CN, C₁₋₆alkyl, —CF₃, —CHF₂, CH₂F, —C₁₋₄alkyl-CF₃, —C₁₋₄alkylCHF₂, —C₁₋₄alkylCH₂F, —OC₁₋₆alkyl, —O—CF₃, —C₁₋₄alkylCF₃, hydroxyC₁₋₆alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NH₂, —NH—C(O)—R⁶, —NH—C(O)—C₃₋₆cycloalkyl, —NH—C(O)—C₁₋₄alkyl, —NH—C(O)—NR⁶R⁷, —NH—S(O)₂—R⁶, —S-heteroaryl, —S-heterocycle, aryl, —C(O)-aryl, heteroaryl, —C(O)-heteroaryl, heterocycle, and —C(O)heterocycle, wherein the aryl, heteroaryl, heteroaryl and heterocycle portion of substituents on choices (1), (2), (3), (4), (5), (6), (7), (8), (9), (22), (23) and (24) is each optionally mono or di-substituted with substituents independently selected from halo, —CH₃, —CF₃, —CN, hydroxy and —OC₁₋₆alkyl, and
wherein the aryl, heteroaryl and heterocycle of choices (10), (11), (12), (13), (14), (15), (16), (17), (18), (19), (20) and (21) is optionally mono-, di- or tri-substituted with substituents independently selected from —CN, oxo, —C₁₋₄alkyl, —C₃₋₆cycloalkyl, —CF₃, —CHF₂, —OC₁₋₆alkyl, —O—CF₃, —O—CH₂—CF₃, hydroxy, -hydroxyC₁₋₆alkyl, halo, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —CH₂-heterocycle, —NR⁶R⁷, —NH—C(O)—R⁶, —NH—C(O)—N(CH₃)₂, —NH—S(O)₂—R⁶, —NH—C₁₋₂alkyl-aryl, and —S—C₁₋₃alkyl, wherein the aryl, heteroaryl and heterocycle portion of the substituents on choices (10), (11), (12), (13), (14), (15), (16), (17), (18), (19), (20) and (21) is each optionally mono or di-substituted with substituents independently selected from halo, —CH₃, —CF₃, —CN, hydroxy and —OC₁₋₄alkyl.

9. A compound of claim 8 wherein
R³ is selected from the group consisting of:
(1) —C(O)—C₁₋₆alkyl,
(2) —C(O)—C₂₋₆alkenyl,
(3) —C(O)—C₂₋₆alkynyl,
(4) —C(O)—C₃₋₆cycloalkyl,
(5) —C(O)-heterocycle,
(6) —C(O)—C₁₋₂alkyl-heterocycle,
(7) —C(O)-heteroaryl,
(8) —C(O)—C₁₋₂alkyl-heteroaryl,
(9) —C(O)-aryl,
(10) —C(O)—C₁₋₂alkyl-aryl,
(11) —C(O)-carbocycle,
wherein the alkyl, cycloalkyl, carbocycle, alkenyl and alkynyl of choices (1), (2), (3), (4), and (11), is each optionally mono- or di-substituted with substituents independently selected from oxo, hydroxy, —CN, —CF₃, —C₁₋₄alkyl-CF₃, —C(O)—O—C₁₋₆alkyl, —C(O)—NR⁶R⁷, and
wherein the aryl, heteroaryl and heterocycle of choices (5), (6), (7), (8), (9) and (10) is optionally mono-, di- or tri-substituted with substituents independently selected from —CN, —C₁₋₄alkyl, —C₃₋₆cycloalkyl, —CF₃, halo, —C(O)—O—C₁₋₆alkyl, —C(O)—NHC₁₋₆alkyl, —C(O)—N(C₁₋₆alkyl)₂, —NR⁶R⁷, —NH—C(O)—R⁶, —NH—C(O)—N(CH₃)₂.

10. A compound of claim 1 wherein
R⁴ and R⁵ are each independently selected from
(1) H,
(2) —C₁₋₆alkyl,
(3) —C₃₋₆cycloalkyl,
(4) heterocycle,
(5) heteroaryl,
(6) aryl,
wherein the alkyl, cycloalkyl, alkenyl and alkynyl of choices (2) and (3) is each independently optionally mono- or di-substituted with substituents independently selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —C(O)—O—C₁₋₆alkyl, —C(O)—NHC₁₋₆alkyl, —C(O)—NR⁶R⁷, —C(O)—O—C(CH₃)₃, —C₃₋₆cycloalkyl, —NH₂, —NHR⁴, —NR⁴R⁵, —NH—C(O)—R⁶, —NH—C(O)—NR⁶R⁷, —NH—S(O)₂—R⁶, and
the heterocycle, heteroaryl and aryl of choices (4), (5), and (6), are each optionally mono- or di-substituted with substituents selected from halo, —CN, hydroxy, oxo, —C₁₋₄alkyl, —C₃₋₆cycloalkyl, —CF₃, —CHF₂, —CH₂F, —OC₁₋₆alkyl, —O—CF₃, —O—C₁₋₃alkyl-CF₃, -hydroxyC₁₋₆alkyl, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —C₁₋₂alkyl-heterocycle, —NR⁶R⁷, —NH—C(O)—NR⁶R⁷, —NH—S(O)₂—R⁶, —NH—C₁₋₄alkyl-aryl, and —S—C₁₋₄alkyl, wherein the aryl, heteroaryl and heterocycle portion of the substituents on choices (9), (10) and (11) are each optionally mono or di-substituted with substituents independently selected from halo, —CH₃, —CF₃, —CN, hydroxy and —OC₁₋₄alkyl.

11. A compound of claim 1 wherein
R⁶ is selected from hydrogen, C₁₋₄alkyl, C₃₋₆cycloalkyl, and aryl.

12. A compound of claim 1 of formula I wherein 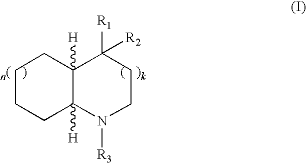
R¹ is independently selected from
(1) H,
(2) OH,
(3) —NH₂,
(4) —NHR⁴,
(5) —NR⁴R⁵,
(6) —CF₃,
(7) —CN,
(8) heterocycle,
(9) heteroaryl,
(10) aryl,
wherein
the heterocycle, heteroaryl and aryl of choices (8), (9), and (10), are each optionally mono- or di-substituted with substituents selected from halo, —CN, —C₁₋₄alkyl, —C₃₋₆cycloalkyl, —CF₃, —OC₁₋₆alkyl, —O—CF₃, —O—C₁₋₃ alkyl-CF₃, —C(O)—O—C₁₋₆alkyl, —C(O)—NR⁶R⁷;
R² is selected from the group consisting of:
(1) —C₁₋₆alkyl,
(2) —C₃₋₆cycloalkyl,
(3) carbocycle,
(4) -aryl,
(5) heterocycle,
(6) heteroaryl,
wherein the alkyl, cycloalkyl, and carbocycle of choices (1), (2) and (3), are each optionally mono- or di-substituted with substituents independently selected from hydroxy, —CF₃, —C₁₋₄alkylCF₃, aryl, heteroaryl, and
wherein the aryl, heteroaryl and heterocycle of choices (4), (5) and (6) are optionally mono-, di- or tri-substituted with substituents independently selected from —CN, halo, —CF₃, —CO₂H, CO₂R⁴, —C(O)NR⁴R⁵, aryl, heteroaryl, C₃₋₆cycloalkyl; or
R¹ and R² are joined so that together with the carbon to which they are attached there is formed a cycloalkyl, carbocyclic or heterocyclic ring, said ring being optionally mono- or di-substituted with a substituent selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, -hydroxyC₁₋₄alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NR⁴R⁵, —NH—C(O)—R⁶, —NH—C(O)—NR⁶R⁷, —NH—S(O)₂—R⁶, heteroaryl, aryl, heterocycle;
R³ is selected from the group consisting of:
(1) —C₁₋₆alkyl,
(2) —C(O)—C₁₋₆alkyl,
(3) —CH₂—C₂₋₆alkenyl,
(4) —C(O)—C₂₋₆alkenyl,
(5) —CH₂—C₂₋₆alkynyl,
(6) —C(O)—C₂₋₆alkynyl,
(7) C₃₋₆cycloalkyl,
(8) —C(O)—C₃₋₆cycloalkyl,
(9) —C(O)N(R⁵)—C₃₋₆cycloalkyl,
(10) heterocycle,
(11) —CH₂-heterocycle,
(12) —C(O)-heterocycle,
(13) —C(O)—C₁₋₂alkyl-heterocycle,
(14) heteroaryl,
(15) —CH₂-heteroaryl,
(16) —C(O)-heteroaryl,
(17) —C(O)—C₁₋₂alkyl-heteroaryl,
(18) aryl,
(19) —CH₂-aryl,
(20) —C(O)-aryl,
(21) —C(O)—C₁₋₂alkyl-aryl,
(22) carbocycle,
(23) —CH₂-carbocycle,
(24) —C(O)-carbocycle,
wherein the alkyl, cycloalkyl, carbocycle, alkenyl and alkynyl of choices (1), (2), (3), (4), (5), (6), (7), (8), (9), (22), (23) and (24) are each optionally mono- or di-substituted with substituents independently selected from halo, oxo, hydroxy, —CN, —C₁₋₆alkyl, —CF₃, —CHF₂, CH₂F, —C₁₋₄alkyl-CF₃, —C₁₋₄alkylCHF₂, —C₁₋₄alkylCH₂F, —O—CF₃, —C₁₋₄alkylCF₃, hydroxyC₁₋₆alkyl, —S(O)₂—R⁶, —C(O)—O—C₁₋₆alkyl, —C(O)—NR⁶R⁷, —C(O)—O—C(CH₃)₃, —C₃₋₆cycloalkyl, —NH₂, —NH—C(O)—R⁶, NH—C(O)—C₃₋₆cycloalkyl, —NH—C(O)—C₁₋₄alkyl, —NH—C(O)—NR⁶R⁷, —NH—S(O)₂—R⁶, —S-heteroaryl, —S-heterocycle, aryl, —C(O)-aryl, heteroaryl, —C(O)-heteroaryl, heterocycle, and —C(O)heterocycle, wherein the aryl, heteroaryl, heteroaryl and heterocycle portion of substituents on choices (1), (2), (3), (4), (5), (6), (7), (8), (9), (22), (23) and (24) is each optionally mono or di-substituted with substituents independently selected from halo, —CH₃, —CF₃, —CN, hydroxy and —OC₁₋₆alkyl, and
wherein the aryl, heteroaryl and heterocycle of choices (10), (11), (12), (13), (14), (15), (16), (17), (18), (19), (20) and (21) is optionally mono-, di- or tri-substituted with substituents independently selected from —CN, oxo, —C₁₋₄alkyl, —C₃₋₆cycloalkyl, —CF₃, —CHF₂, —OC₁₋₆alkyl, —O—CF₃, —O—CH₂—CF₃, hydroxy, -hydroxyC₁₋₆alkyl, halo, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —CH₂-heterocycle, —NR⁶R⁷, —NH—C(O)—R⁶, —NH—C(O)—N(CH₃)₂, —NH—S(O)₂—R⁶, —NH—C₁₋₂alkyl-aryl, and —S—C₁₋₃alkyl, wherein the aryl, heteroaryl and heterocycle portion of the substituents on choices (10), (11), (12), (13), (14), (15), (16), (17), (18), (19), (20) and (21) is each optionally mono or di-substituted with substituents independently selected from halo, —CH₃, —CF₃, —CN, hydroxy and —OC₁₋₄alkyl;
R⁴ and R⁵ are each independently selected from
(1) H,
(2) —C₁₋₆alkyl,
(3) —C₃₋₆cycloalkyl,
(4) heterocycle,
(5) heteroaryl,
(6) aryl,
wherein the alkyl, cycloalkyl, alkenyl and alkynyl of choices (2) and (3) is each independently optionally mono- or di-substituted with substituents independently selected from hydroxy, oxo, halo, —C₁₋₆alkyl, —CF₃, —CHF₂, —CH₂F, —C₁₋₄alkylCF₃, —C₁₋₄alkylCHF₂, —C₁₋₄alkylCH₂F, —OC₁₋₆alkyl, —O—CF₃, —O—CHF₂, —O—CH₂F, —O—C₁₋₄alkyl-CF₃, —O—C₁₋₄alkylCHF₂, —O—C₁₋₄alkylCH₂F, —C₁₋₄alkyl-OH, —S(O)₂—R⁶, —C(O)—O—C₁₋₆alkyl, —C(O)—NHC₁₋₆alkyl, —C(O)—NR⁶R⁷, —C(O)—O—C(CH₃)₃, —C₃₋₆cycloalkyl, —NH₂, —NHR⁴, —NR⁴R⁵, —NH—C(O)—R⁶, —NH—C(O)—NR⁶R⁷, —NH—S(O)₂—R⁶, and
the heterocycle, heteroaryl and aryl of choices (4), (5), and (6), are each optionally mono- or di-substituted with substituents selected from halo, —CN, hydroxy, oxo, —C₁₋₄alkyl, —C₃₋₆cycloalkyl, —CF₃, —CHF₂, —CH₂F, —OC₁₋₆alkyl, —O—CF₃, —O—C₁₋₃alkyl-CF₃, -hydroxyC₁₋₆alkyl, —S(O)₂—R⁶, —C(O)—O—C₁₋₆alkyl, —C(O)—NHC₁₋₆alkyl, —C(O)—N(C₁₋₆alkyl)₂, —C(O)—O—C(CH₃)₃, aryl, —C(O)aryl, —C₁₋₂alkyl-aryl, heteroaryl, —C(O)-heteroaryl, —C₁₋₂alkyl-heteroaryl, —C₃₋₆cycloalkyl, heterocycle, —C(O)-heterocycle, —C₁₋₂alkyl-heterocycle, —NR⁶R⁷, —NH—C(O)—NR⁶R⁷, —NH—S(O)₂—R⁶, —NH—C₁₋₄alkyl-aryl, and —S—C₁₋₄alkyl, wherein the aryl, heteroaryl and heterocycle portion of the substituents on choices (9), (10) and (11) are each optionally mono or di-substituted with substituents independently selected from halo, —CH₃, —CF₃, —CN, hydroxy and —OC₁₋₄alkyl;
R⁶ is selected from hydrogen, C₁₋₄alkyl, C₃₋₆cycloalkyl, and aryl.

13. A compound of claim 12 wherein
R¹ is independently selected from
(1) H,
(2) OH,
(3) —NH₂,
(6) —CF₃,
(7) —CN;
R³ is selected from the group consisting of:
(1) —C(O)—C₁₋₆alkyl,
(2) —C(O)—C₂₋₆alkenyl,
(3) —C(O)—C₂₋₆alkynyl,
(4) —C(O)—C₃₋₆cycloalkyl,
(5) —C(O)-heterocycle,
(6) —C(O)—C₁₋₂alkyl-heterocycle,
(7) —C(O)-heteroaryl,
(8) —C(O)—C₁₋₂alkyl-heteroaryl,
(9) —C(O)-aryl,
(10) —C(O)—C₁₋₂alkyl-aryl,
(11) —C(O)-carbocycle,
wherein the alkyl, cycloalkyl, carbocycle, alkenyl and alkynyl of choices (1), (2), (3), (4), and (11), is each optionally mono- or di-substituted with substituents independently selected from oxo, hydroxy, —CN, —CF₃, —C₁₋₄alkyl-CF₃, —C(O)—NR⁶R⁷, and
wherein the aryl, heteroaryl and heterocycle of choices (5), (6), (7), (8), (9) and (10) is optionally mono-, di- or tri-substituted with substituents independently selected from —CN, —C₁₋₄alkyl, —C₃₋₆cycloalkyl, —CF₃, halo, —C(O)—O—C₁₋₆alkyl, —C(O)—NHC₁₋₆alkyl, —C(O)—N(C₁₋₆alkyl)₂, —NR⁶R⁷, —NH—C(O)—R⁶, —NH—C(O)—N(CH₃)₂.

14. A compound according to claim 1 selected from the group consisting of

15. A pharmaceutical composition comprising a compound of claim 1 and a pharmaceutically acceptable carrier.

16. A method of modulating the CB2 receptor in a patient in need of such modulation, comprising administering an effective amount of a compound according to claim 1.

17. A method of agonizing the CB2 receptor in a patient in need of such agonizing, comprising administering an effective amount of a compound according to claim 1.

18. A method of treating a disease mediated by agonizing the CB2 receptor in a patient in need of such treatment, comprising administering an effective amount of a compound according to claim 1.

19. A method of treating a disease selected from the group consisting inflammatory pain, osteoporosis, atheroschlerosis, immune disorders and arthritis comprising administering an effective amount of a compound according to claim 1.

20. A method according to claim 19, for the treatment of the acute and chronic pain.

21. A method according to claim 21, for the treatment of the pain of rheumatoid arthritis or osteoarthritis.
